SEQUENCE LISTING (11 sequences)

SEQ ID NO: 1 - DNA, 1392 bp, Clostridium tetani
atggttttttcaacaccaattccattttcttattctaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaaccctttacgatatgatacagaatattatttaataccagtagcttctagttctaaagatgttcaattgaaaaatataacagattatatgtatttgacaaatgcgccatcgtatactaacggaaaattgaatatatattatagaaggttatataatggactaaaatttattataaaaagatatacacctaataatgaaatagattcttttgttaaatcaggtgattttattaaattatatgtatcatataacaataatgagcacattgtaggttatccgaaagatggaaatgcctttaataatcttgatagaattctaagagtaggttataatgccccaggtatccctctttataaaaaaatggaagcagtaaaattgcgtgatttaaaaacctattctgtacaacttaaattatatgatgataaaaatgcatctttaggactagtaggtacccataatggtcaaataggcaacgatccaaatagggatatattaattgcaagcaactggtactttaatcatttaaaagataaaattttaggatgtgattggtactttgtacctacagatgaaggatggacaaatgattaa

SEQ ID NO: 2 - protein, 462 aa, Clostridium tetani
VFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 3 - DNA, 21 bp, Artificial Sequence
  (Primer)
agattccgcgggagaagttag

SEQ ID NO: 4 - DNA, 21 bp, Artificial Sequence
  (Primer)
tcgtaaagggtttccccagaa

SEQ ID NO: 5 - protein, 451 aa, Clostridium tetani
  MISC_FEATURE (1)...(451): Fragment of SEQ ID NO2
KNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 6 - DNA, 1359 bp, Clostridium tetani
atgaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaaccctttacgatatgatacagaatattatttaataccagtagcttctagttctaaagatgttcaattgaaaaatataacagattatatgtatttgacaaatgcgccatcgtatactaacggaaaattgaatatatattatagaaggttatataatggactaaaatttattataaaaagatatacacctaataatgaaatagattcttttgttaaatcaggtgattttattaaattatatgtatcatataacaataatgagcacattgtaggttatccgaaagatggaaatgcctttaataatcttgatagaattctaagagtaggttataatgccccaggtatccctctttataaaaaaatggaagcagtaaaattgcgtgatttaaaaacctattctgtacaacttaaattatatgatgataaaaatgcatctttaggactagtaggtacccataatggtcaaataggcaacgatccaaatagggatatattaattgcaagcaactggtactttaatcatttaaaagataaaattttaggatgtgattggtactttgtacctacagatgaaggatggacaaatgattaa

SEQ ID NO: 7 - DNA, 1402 bp, Artificial Sequence
  humanized tetanus toxin C fragment
ggcggaggtaccgtcgacctcgaggaaagaacctggactgctgggtggacaacgaggaggacatcgacgtgatcctgaagaagagcaccatcctgaacctggacatcaacaacgacatcatcagcgacatcagcggcttcaacagcagcgtgatcacctaccccgacgcccagctggtgcccggcatcaacggcaaggccatccacctggtgaacaacgagagcagcgaggtgatcgtgcacaaggccatggacatcgagtacaacgacatgttcaacaacttcaccgtgagcttctggctgagagtgcccaaggtgagcgccagccacctggagcagtacgacaccaacgagtacagcatcatcagcagcatgaagaagtacagcctgagcatcggcagcggctggagcgtgagcctgaagggcaacaacctgatctggaccctgaaggacagcgccggcgaggtgagacagatcaccttcagagacctgagcgacaagttcaacgcctacctggccaacaagtgggtgttcatcaccatcaccaacgacagactgagcagcgccaacctgtacatcaacggcgtgctgatgggcagcgccgagatcaccggcctgggcgccatcagagaggacaacaacatcaccctgaagctggacagatgcaacaacaacaaccagtacgtgagcatcgacaagttcagaatcttctgcaaggccctgaaccccaaggagatcgagaagctgtacaccagctacctgagcatcaccttcctgagagacttctggggcaaccccctgagatacgacaccgagtactacctgatccccgtggcctacagcagcaaggacgtgcagctgaagaacatcaccgactacatgtacctgaccaacgcccccagctacaccaacggcaagctgaacatctactacagaagactgtacagcggcctgaagttcatcatcaagagatacacccccaacaacgagatcgacagcttcgtgagaagcggcgacttcatcaagctgtacgtgagctacaacaacaacgagcacatcgtgggctaccccaaggacggcaacgccttcaacaacctggacagaatcctgagagtgggctacaacgcccccggcatccccctgtacaagaagatggaggccgtgaagctgagagacctgaagacctacagcgtgcagctgaagctgtacgacgacaaggacgccagcctgggcctggtgggcacccacaacggccagatcggcaacgaccccaacagagacatcctgatcgccagcaactggtacttcaaccacctgaaggacaagaccctgacctgcgactggtacttcgtgcccaccgacgagggctggaccaacgactgactcgagggaggcgccggcgg

SEQ ID NO: 8 - DNA, 1356 bp, Artificial Sequence
  humanized tetanus toxin C fragment
aagaacctggactgctgggtggacaacgaggaggacatcgacgtgatcctgaagaagagcaccatcctgaacctggacatcaacaacgacatcatcagcgacatcagcggcttcaacagcagcgtgatcacctaccccgacgcccagctggtgcccggcatcaacggcaaggccatccacctggtgaacaacgagagcagcgaggtgatcgtgcacaaggccatggacatcgagtacaacgacatgttcaacaacttcaccgtgagcttctggctgagagtgcccaaggtgagcgccagccacctggagcagtacgacaccaacgagtacagcatcatcagcagcatgaagaagtacagcctgagcatcggcagcggctggagcgtgagcctgaagggcaacaacctgatctggaccctgaaggacagcgccggcgaggtgagacagatcaccttcagagacctgagcgacaagttcaacgcctacctggccaacaagtgggtgttcatcaccatcaccaacgacagactgagcagcgccaacctgtacatcaacggcgtgctgatgggcagcgccgagatcaccggcctgggcgccatcagagaggacaacaacatcaccctgaagctggacagatgcaacaacaacaaccagtacgtgagcatcgacaagttcagaatcttctgcaaggccctgaaccccaaggagatcgagaagctgtacaccagctacctgagcatcaccttcctgagagacttctggggcaaccccctgagatacgacaccgagtactacctgatccccgtggcctacagcagcaaggacgtgcagctgaagaacatcaccgactacatgtacctgaccaacgcccccagctacaccaacggcaagctgaacatctactacagaagactgtacagcggcctgaagttcatcatcaagagatacacccccaacaacgagatcgacagcttcgtgagaagcggcgacttcatcaagctgtacgtgagctacaacaacaacgagcacatcgtgggctaccccaaggacggcaacgccttcaacaacctggacagaatcctgagagtgggctacaacgcccccggcatccccctgtacaagaagatggaggccgtgaagctgagagacctgaagacctacagcgtgcagctgaagctgtacgacgacaaggacgccagcctgggcctggtgggcacccacaacggccagatcggcaacgaccccaacagagacatcctgatcgccagcaactggtacttcaaccacctgaaggacaagaccctgacctgcgactggtacttcgtgcccaccgacgagggctggaccaacgactga

SEQ ID NO: 9 - RNA, 1392 nt, Clostridium tetani
augguuuuuucaacaccaauuccauuuucuuauucuaaaaaucuggauuguuggguugauaaugaagaagauauagauguuauauuaaaaaagaguacaauuuuaaauuuagauauuaauaaugauauuauaucagauauaucuggguuuaauucaucuguaauaacauauccagaugcucaauuggugcccggaauaaauggcaaagcaauacauuuaguaaacaaugaaucuucugaaguuauagugcauaaagcuauggauauugaauauaaugauauguuuaauaauuuuaccguuagcuuuugguugaggguuccuaaaguaucugcuagucauuuagaacaauauggcacaaaugaguauucaauaauuagcucuaugaaaaaacauagucuaucaauaggaucugguuggaguguaucacuuaaagguaauaacuuaauauggacuuuaaaagauuccgcgggagaaguuagacaaauaacuuuuagggauuuaccugauaaauuuaaugcuuauuuagcaaauaaauggguuuuuauaacuauuacuaaugauagauuaucuucugcuaauuuguauauaaauggaguacuuaugggaagugcagaaauuacugguuuaggagcuauuagagaggauaauaauauaacauuaaaacuagauagauguaauaauaauaaucaauacguuucuauugauaaauuuaggauauuuugcaaagcauuaaauccaaaagagauugaaaaauuauacacaaguuauuuaucuauaaccuuuuuaagagacuucuggggaaacccuuuacgauaugauacagaauauuauuuaauaccaguagcuucuaguucuaaagauguucaauugaaaaauauaacagauuauauguauuugacaaaugcgccaucguauacuaacggaaaauugaauauauauuauagaagguuauauaauggacuaaaauuuauuauaaaaagauauacaccuaauaaugaaauagauucuuuuguuaaaucaggugauuuuauuaaauuauauguaucauauaacaauaaugagcacauuguagguuauccgaaagauggaaaugccuuuaauaaucuugauagaauucuaagaguagguuauaaugccccagguaucccucuuuauaaaaaaauggaagcaguaaaauugcgugauuuaaaaaccuauucuguacaacuuaaauuauaugaugauaaaaaugcaucuuuaggacuaguagguacccauaauggucaaauaggcaacgauccaaauagggauauauuaauugcaagcaacugguacuuuaaucauuuaaaagauaaaauuuuaggaugugauugguacuuuguaccuacagaugaaggauggacaaaugauuaa

SEQ ID NO: 10 - RNA, 1359 nt, Clostridium tetani
augaaaaaucuggauuguuggguugauaaugaagaagauauagauguuauauuaaaaaagaguacaauuuuaaauuuagauauuaauaaugauauuauaucagauauaucuggguuuaauucaucuguaauaacauauccagaugcucaauuggugcccggaauaaauggcaaagcaauacauuuaguaaacaaugaaucuucugaaguuauagugcauaaagcuauggauauugaauauaaugauauguuuaauaauuuuaccguuagcuuuugguugaggguuccuaaaguaucugcuagucauuuagaacaauauggcacaaaugaguauucaauaauuagcucuaugaaaaaacauagucuaucaauaggaucugguuggaguguaucacuuaaagguaauaacuuaauauggacuuuaaaagauuccgcgggagaaguuagacaaauaacuuuuagggauuuaccugauaaauuuaaugcuuauuuagcaaauaaauggguuuuuauaacuauuacuaaugauagauuaucuucugcuaauuuguauauaaauggaguacuuaugggaagugcagaaauuacugguuuaggagcuauuagagaggauaauaauauaacauuaaaacuagauagauguaauaauaauaaucaauacguuucuauugauaaauuuaggauauuuugcaaagcauuaaauccaaaagagauugaaaaauuauacacaaguuauuuaucuauaaccuuuuuaagagacuucuggggaaacccuuuacgauaugauacagaauauuauuuaauaccaguagcuucuaguucuaaagauguucaauugaaaaauauaacagauuauauguauuugacaaaugcgccaucguauacuaacggaaaauugaauauauauuauagaagguuauauaauggacuaaaauuuauuauaaaaagauauacaccuaauaaugaaauagauucuuuuguuaaaucaggugauuuuauuaaauuauauguaucauauaacaauaaugagcacauuguagguuauccgaaagauggaaaugccuuuaauaaucuugauagaauucuaagaguagguuauaaugccccagguaucccucuuuauaaaaaaauggaagcaguaaaauugcgugauuuaaaaaccuauucuguacaacuuaaauuauaugaugauaaaaaugcaucuuuaggacuaguagguacccauaauggucaaauaggcaacgauccaaauagggauauauuaauugcaagcaacugguacuuuaaucauuuaaaagauaaaauuuuaggaugugauugguacuuuguaccuacagaugaaggauggacaaaugauuaa

SEQ ID NO: 11 - RNA, 1356 nt, Artificial Sequence
  humanized tetanus toxin C fragment
aagaaccuggacugcuggguggacaacgaggaggacaucgacgugauccugaagaagagcaccauccugaaccuggacaucaacaacgacaucaucagcgacaucagcggcuucaacagcagcgugaucaccuaccccgacgcccagcuggugcccggcaucaacggcaaggccauccaccuggugaacaacgagagcagcgaggugaucgugcacaaggccauggacaucgaguacaacgacauguucaacaacuucaccgugagcuucuggcugagagugcccaaggugagcgccagccaccuggagcaguacgacaccaacgaguacagcaucaucagcagcaugaagaaguacagccugagcaucggcagcggcuggagcgugagccugaagggcaacaaccugaucuggacccugaaggacagcgccggcgaggugagacagaucaccuucagagaccugagcgacaaguucaacgccuaccuggccaacaaguggguguucaucaccaucaccaacgacagacugagcagcgccaaccuguacaucaacggcgugcugaugggcagcgccgagaucaccggccugggcgccaucagagaggacaacaacaucacccugaagcuggacagaugcaacaacaacaaccaguacgugagcaucgacaaguucagaaucuucugcaaggcccugaaccccaaggagaucgagaagcuguacaccagcuaccugagcaucaccuuccugagagacuucuggggcaacccccugagauacgacaccgaguacuaccugauccccguggccuacagcagcaaggacgugcagcugaagaacaucaccgacuacauguaccugaccaacgcccccagcuacaccaacggcaagcugaacaucuacuacagaagacuguacagcggccugaaguucaucaucaagagauacacccccaacaacgagaucgacagcuucgugagaagcggcgacuucaucaagcuguacgugagcuacaacaacaacgagcacaucgugggcuaccccaaggacggcaacgccuucaacaaccuggacagaauccugagagugggcuacaacgcccccggcaucccccuguacaagaagauggaggccgugaagcugagagaccugaagaccuacagcgugcagcugaagcuguacgacgacaaggacgccagccugggccuggugggcacccacaacggccagaucggcaacgaccccaacagagacauccugaucgccagcaacugguacuucaaccaccugaaggacaagacccugaccugcgacugguacuucgugcccaccgacgagggcuggaccaacgacuga

(19) United States
(12) Patent Application Publication
(10) Pub. No.: US 2017/0151316 A1
(43) Pub. Date: Jun. 1, 2017

(54) METHODS OF INCREASING MUSCLE MASS USING NON-TOXIC TETANUS TOXIN C FRAGMENT (TTC)

(71) Applicants: SPHERIUM BIOMED, S.L., Espluges de Llobregat, Barcelona (ES); UNIVERSIDAD DE ZARAGOZA, Zaragoza (ES)

(72) Inventors: Jordi ORTIZ SAGRISTA, Barcelona (ES); Ramon BOSSER ARTAL, Barcelona (ES); Laura MORENO MARTINEZ, Zaragoza (ES); Ana Cristina CALVO ROYO, Zaragoza (ES); Maria Jesus MUÑOZ GONZALVO, Zaragoza (ES); Pilar ZARAGOZA FERNANDEZ, Zaragoza (ES); Rosario OSTA PINZOLAS, Zaragoza (ES)

(73) Assignee: SPHERIUM BIOMED, S.L., Espluges de Llobregat, Barcelona (ES)

(21) Appl. No.: 15/322,777

(22) PCT Filed: Jul. 1, 2015
(86) PCT No.: PCT/IB2015/001705

§ 371 Date: Dec. 29, 2016

Related U.S. Application Data
(60) Provisional application No. 62/020,237, filed on Jul. 2, 2014.

(51) Int. Cl. A61K 38/48; A61K 48/00

(52) U.S. Cl. CPC A61K 38/4886; C12Y 304/24068; A61K 48/005

(57) ABSTRACT

The present disclosure relates to the use of the non-toxic proteolytic C fragment of tetanus toxin and plasmids encoding such protein fragment to increase muscle mass and/or muscle strength in a subject in need thereof. As such, methods of ameliorating the severity of a pathological condition characterized, at least in part, by a decreased amount, development, or metabolic activity of muscle are provided. The disclosed compositions and method are also useful for the treatment of condition in which increase in muscle mass and muscle strength are desirable, including cosmetic uses.

### REFERENCE TO SEQUENCE LISTING SUBMITTED ELECTRONICALLY

The content of the electronically submitted sequence listing in ASCII text file (Name: TTC_SequenceListing_ascii.txt; Size: 21,620 bytes; and Date of Creation: Jun. 25, 2015) filed with the application is incorporated herein by reference in its entirety.

### BACKGROUND

A number of human and animal disorders are associated with loss of or functionally impaired muscle tissue or muscle wasting, for example neuromuscular disorders, muscular dystrophies, or HIV-infection. Muscle wasting in patients while at bed rest is a huge and common clinical issue. It is known that patients in intensive care units become catabolic, that is, tear down muscle tissue, almost immediately after confinement. Significant loss of muscle has been shown even in healthy, young volunteers whose leg has been immobilized a cast for only two weeks. See, e.g., Hespel et al. J. Physiol. 536:625-633 (2001). Extreme loss of muscle tissue leads to a condition termed cachexia, which is often seen in cancer, trauma and burn patients.

Loss of muscle mass can occur as a normal part of aging and extraordinary measures are necessary to stave it off and shift the metabolism to a more anabolic state. Such muscle loss and/or loss of muscle tone can also have important cosmetic effects.

Athletes also can benefit from enhanced muscle development. In their training, especially in weight or cardiovascular training intense enough to reach the anaerobic threshold, they are constantly tearing down muscle fiber (catabolism) and rebuilding the fibers (anabolism).

To date, very few reliable or effective treatment methods exist to promote muscle growth (i.e., to increase muscle mass or volume) and/or increase muscle strength. While not curing the conditions associated with muscle loss, therapies that increase the amount of muscle tissue in patients suffering from such disorders would significantly improve the quality of life for these patients and could ameliorate some of the effects of these diseases. Thus, there is a need in the art to identify new therapies that may contribute to an overall increase in muscle tissue, or prevent muscle loss in patients suffering from these conditions, diseases or disorders.

### BRIEF SUMMARY

The present disclosure provides method of treating a disease or condition associated with decreased muscle mass and/or muscle strength in a subject in need thereof comprising administering a therapeutically effective amount of non-toxic tetanus toxin C fragment (TTC) to the subject, wherein said administration is effective to (i) increase muscle mass in the subject, and/or (ii) increase muscle strength in the subject, and/or (iii) increase the rate of recovery or healing, and/or (iv) decrease fibrosis caused by said disease or condition.

The methods disclosed herein can also treat, prevent, or ameliorate the loss of muscle mass; decrease the rate of loss of muscle mass; treat, prevent, or ameliorate the symptoms associated with the loss of muscle mass; treat, prevent, or ameliorate the loss of muscle strength; treat, prevent, or ameliorate the symptoms associated with the loss of muscle strength; treat, prevent, or ameliorate fibrosis caused by the disease or condition; or treat, prevent, or ameliorate the symptoms associated with the fibrosis caused by the disease or condition. In some aspects, the disease or condition is a wasting disorder. In some aspects, the wasting disorder is selected from the group consisting of cachexia and anorexia. In other aspects, the wasting disorder is selected from the group consisting of a muscular dystrophy and a neuromuscular disease. In some aspects, the condition is a sequelae of immobilization, chronic disease, cancer, or injury.

Also provided is a method of increasing muscle mass in a subject in need thereof comprising administering TTC to the subject. In some aspects, the increase in muscle mass is to compensate for wasting resulting from a wasting disorder, immobilization, or old age. In other aspects, the increase in muscle mass is for cosmetic purposes.

The present disclosure also provides a method of treating a patient having a disease or condition associated with loss of muscle mass and/or loss of muscle strength comprising administering a therapeutically effective amount of TTC to the subject if the level of Col19a1 (Collagen alpha-1(XIX) chain) and/or Snx10 (Sorting Nexin 10) in a sample taken from the patient is above a predetermined Col19a1 and/or Snx10 threshold level, or is above the Col19a1 and/or Snx10 level in one or more control samples, wherein said administration is effective to (i) increase muscle mass, and/or (ii) increase muscle strength, and/or (iii) increase the rate of recovery or healing, and/or (iv) decrease fibrosis caused by said disease or condition in the subject.

Also provided is method of treating a patient having a disease or condition associated with loss of muscle mass and/or loss of muscle strength comprising (a) submitting a sample taken from the patient for measurement of the level of Col19a1 and/or Snx10, and (b) administering a therapeutically effective amount of TTC to the subject if the level of Col19a1 and/or Snx10 in the sample taken from the patient is above a predetermined Col19a1 and/or Snx10 threshold level, or is above the Col19a1 and/or Snx10 level in one or more control samples, wherein said administration is effective to (i) increase muscle mass, and/or (ii) increase muscle strength, and/or (iii) increase the rate of recovery or healing, and/or (iv) decrease fibrosis caused by said disease or condition in the subject.

In addition, the present disclosure provides a method of treating a patient having a disease or condition associated with loss of muscle mass and/or loss of muscle strength comprising (a) measuring the level of Col19a1 and/or Snx10 submitting a sample taken from the patient, (b) determining whether the patient's level of Col19a1 and/or Snx10 is above a predetermined Col19a1 and/or Snx10 threshold level, or is above the Col19a1 and/or Snx10 level in one or more control samples, and (c) advising a healthcare provider to administer a therapeutically effective amount of TTC to the subject, wherein said administration is effective to (i) increase muscle mass, and/or (ii) increase muscle strength, and/or (iii) increase the rate of recovery or healing, and/or (iv) decrease fibrosis caused by said disease or condition in the subject.

Also provided is method of determining whether to treat a patient diagnosed with a disease or condition associated with loss of muscle mass and/or loss of muscle strength comprising (a) measuring, or instructing a clinical laboratory to measure the level of Col19a1 and/or Snx10 in a sample obtained from the patient; and (b) treating, or instructing a healthcare provider to treat, the patient by administering TTC if the patient's level of Col19a1 and/or Snx10 in the sample is above a predetermined Col19a1 and/or Snx10 threshold level, or is above the level of Col19a1 and/or Snx10 in one or more control samples, wherein said administration is effective to (i) increase muscle mass, and/or (ii) increase muscle strength, and/or (iii) increase the rate of recovery or healing, and/or (iv) decrease fibrosis caused by said disease or condition in the subject.

The disclosure provides also method of selecting a patient diagnosed with a disease or condition associated with loss of muscle mass and/or loss of muscle strength as a candidate for treatment with a TTC therapeutic regimen comprising (a) measuring, or instructing a clinical laboratory to measure the level of Col19a1 and/or Snx10 in a sample obtained from the patient; and (b) treating, or instructing a healthcare provider to treat the patient by administering TTC if the patient's level of Col19a1 and/or Snx10 in the sample is above a predetermined Col19a1 and/or Snx10 threshold level, or is above the level of Col19a1 and/or Snx10 in one or more control samples, wherein said administration is effective to (i) increase muscle mass, and/or (ii) increase muscle strength, and/or (iii) increase the rate of recovery or healing, and/or (iv) decrease fibrosis caused by said disease or condition in the subject.

In some aspects, the sample taken from the patient comprises muscle tissue. In some aspects, the subject is human. In other aspects, TTC comprises:

(a) a polypeptide comprising the sequence of SEQ ID NO:2 or SEQ ID NO:5, or a fragment, variant, or derivative thereof;

(b) a polynucleotide comprising the sequence of SEQ ID NO:1 or SEQ ID NO:6, or a fragment, variant, or derivative thereof; or,

(c) combinations thereof.

In other aspects, TTC comprises:

(a) a fusion protein or conjugate wherein a TTC polypeptide is the only therapeutic moiety;

(b) a fusion protein, or conjugate comprising at least two therapeutic moieties, wherein a TTC polypeptide is one of the therapeutic moieties;

(c) a nucleic acid encoding a fusion protein wherein a TTC polypeptide is the only therapeutic moiety;

(d) a nucleic acid encoding a fusion protein comprising at least two therapeutic moieties, wherein a TTC polypeptide is one of the therapeutic moieties; or,

(e) a combination thereof.

In some aspects, TTC is administered as a naked DNA or RNA. Is some aspects, the DNA or RNA is humanized. In some aspects, the humanized DNA comprises the sequence of SEQ ID NO: 8, or a variant, fragment, or derivative thereof. In some aspects, the RNA is an mRNA. In some aspects, the mRNA is a sequence optimized mRNA. In some aspects, the sequence optimized mRNA comprises pseudouridine (Ψ), 5-methoyxuridine (5moU), 2-thiouridine (s2U), 4-thiouridine (s4U), N1-methylpseudouridine (1mΨ), 5-methylcytidine, or a combination thereof. In some aspects, the mRNA comprises the sequence of SEQ ID NO: 9, SEQ ID NO:10, or SEQ ID NO: 11, or a fragment, variant, or derivative thereof.

In other aspects, TTC is administered at a fixed dose. In some aspects, TTC is administered in two or more doses. In some aspects, TTC is administered daily, weekly, biweekly, or monthly. In other aspects, TTC is administered intramuscularly, intraperitoneally, subcutaneously, intravenously, or a combination thereof. In some aspects, the methods disclosed herein are performed in vivo in a mammal. In some aspects, the methods disclosed herein comprise at least one additional therapy.

In some aspects, the disease or condition is a muscle lesion. In some aspects, the muscle lesion is an acute or a chronic muscle lesion. In some aspects, the muscle lesion is a mechanical lesion, a thermal lesion, a chemical lesion, an occupational or repeated stress lesion, a iatrogenic lesion, an athletic muscle lesion, or a combination thereof.

In some aspects, the muscle lesion is treated by directly injecting a therapeutically effective amount of TTC to the site of the lesion.

### BRIEF DESCRIPTION OF THE DRAWINGS/FIGURES

FIG. 1 shows PCR amplification for the detection of the expression of non-toxic tetanus toxin fragment C (TTC). Ten days after intramuscular injection of the plasmid pCMV-TTC, (n=2, lanes 1 and 2) and of the empty plasmid pCMV (n=2, lanes 3 and 4) the RNA was extracted from the muscle and retrotranscribed. The obtained cDNA was amplified by PCR for the TTC gene. Lane 5 shows the positive control (pCMV-TTC plasmid) and the reaction blank was run in lane 6. Lane M corresponds to a 100 bp size marker.

FIG. 2 shows the effects of the treatment with naked DNA encoding TTC on the start of neuromuscular symptoms in SOD1G93A model mice (model system for ALS, a disease characterized by muscle atrophy, which includes reduction of muscle mass, increase in muscle weakness, and general loss of muscle function). The manifestation of symptoms was significantly delayed in the group treated with TTC (n=10) with respect to the control group (n=10). The accumulated probabilities were calculated using the Kaplan-Meier survival analysis (SPSS 13.0).

FIG. 3 shows the effects of the treatment with naked DNA encoding TTC on the survival of SOD1G93A model mice. Survival notably increased in the group treated with TTC (n=10) in respect of the control group (n=10). The accumulated probabilities were calculated using the Kaplan-Meier survival analysis (SPSS 13.0).

FIG. 4 shows the effects of the treatment with naked DNA encoding TTC. Motor activity was determined using the Rotarod at a constant speed of 14 rpm, with a maximum development time of 180 s. Improved motor activity is observed in the group treated with TTC (n=10) in respect of the control (n=10).

FIG. 5 presents the effect of the intramuscular injection of naked DNA encoding TTC in SOD1G93A mice. The strength and motor function of the mice were tested with the hanging-wire test using 10 mice from each group (n=10).

FIG. 6 presents the effect of the intramuscular injection of naked DNA encoding TTC in SOD1G93A mice. Measurement of the weight of the transgenic mice treated with TTC using 10 mice from each group (n=10).

FIG. 7 shows the analysis of the expression of genes involved in the signaling route of apoptosis in the spinal cord of symptomatic SOD1G93A mice of 110 days of age. Representation of the average values of messenger RNA of genes Casp1, Casp3, Bax and Bcl2 in the control (white) and in the mice treated with TTC (grey). The previous groups of mice were compared with wild-type mice (black) (n=5 mice per group).

FIG. 8 presents analysis of the proteins involved in the signaling route of apoptosis in the spinal cord of symptomatic SOD1G93A mice of 110 days of age. Western-blot analysis of the proteins pro-Casp3, active Casp3, Bax and Bcl2 on spinal cord lysates of mice treated with TTC (grey lines) and control mice (black lines) in relation to wild-type mice (black) (n=5 mice per group).

FIG. 9. shows a Western-blot analysis of the phosphorylation of proteins Akt and Erk1/2. Samples of 5 mice per group were analyzed. IDV (Intensity Density Value). The amounts analyzed using the Western-blot appear as the ratio of beta-tubulin in respect of the values of the wild-type mice. (*P<0.05, **P<0.01; the error bars show SEM). The bars represent the same groups as described in the previous caption.

FIG. 10 shows the effects of intraperitoneal treatment with TTC polypeptide on the survival of SOD1G93A model mice. Survival notably increased in the group treated with TTC (n=3, dotted line) in relation to the control group (n=3, continuous line). The accumulated probabilities were calculated using the Kaplan-Meier survival analysis (SPSS 13.0).

FIG. 11 shows that the intramuscular treatment of mice injected with TTC affects the expression of genes related to the homeostasis of calcium in the spinal cord of transgenic SOD1G93A mice. The levels of expression were determined of genes Ncs1 and Rrad in transgenic mice treated with TTC (grey) or with the empty plasmid (white). The changes in levels of messenger RNA in the groups of mice above were compared to wild-type mice (black). (*P<0.05; the error bars show SEM; n=5 mice per group).

FIG. 12 shows the amplitude of M waves in hind limb muscles of SOD1G93A mice at 12 and 16 weeks of age. Figure panels show representative recordings of M waves recorded from tibialis anterior muscles. FIG. 12A shows recording from a wild-type mouse at 16 weeks of age. FIG. 12B shows recording from a SOD1G93A mouse at 12 weeks of age, and FIG. 12C shows recording from a SOD1G93A mouse at 16 weeks of age. Note the marked decline in amplitude and the slight increase in latency from the stimulus to the onset of the M wave in SOD1G93A mice, abnormalities that progress with time (compare FIG. 12B and FIG. 12C). Squares in the recording are 10 mV in height and lms in width. FIG. 12D is a histogram of the mean CMAP (M wave) amplitude in tibialis anterior and plantar muscles in SOD1G93A mice. The amplitudes were similar in control (vehicle-plasmid mice) and TTC-treated mice (SOD-TTC) at 12 weeks, but declined more markedly in control than in treated mice at 16 weeks. The neurophysiological results are shown on TABLE 3.

FIG. 13 shows motor neuron survival in SOD1G93A mice. FIG. 13A shows microphotographs of MNs from wild type and SOD mice stained with cresyl violet. Note the vacuolization and disintegration of Nissl substance in the SOD1G93A MNs. Bar=40 μm. FIG. 13B shows representative micrographs showing cross-sections of the lumbar spinal cords stained with cresyl violet from a wild type, a control SOD1G93A and a SOD1G93A-TTC (SOD-TTC) treated mouse at 16 weeks of age. Bar=500 μm. FIG. 13C shows motor neuron survival assessed by counting the number of stained (cresyl violet) MNs within the lateral column of each ventral horn. The results show the average numbers of MNs counted in the ventral horns at L2, L3 and L4 spinal cord segments of wild type, control SOD1G93A (vehicle-plasmid mice) and TTC-treated SOD1G93A mice (SOD-TTC) (n=5 per group). *P<0.05 vs. wild type; #P<0.05 vs. control SOD1.

FIG. 14 presents an analysis of glial reactivity in SOD1G93A mice. FIG. 14A shows representative microphotographs of spinal cords ventral horns from a wild type, a SOD1G93A and a SOD1G93A-TTC (SOD-TTC) treated mice immunolabeled with markers for astrocytes (GFAP) and microglia (Ibal). Bar=100 μm. FIG. 14B shows histograms representing the quantification of GFAP and Ibal immunoreactivity (IR) in the three groups of mice. *P<0.05 vs. wild type; #P<0.05 vs. control SOD1.

FIG. 15 presents a schematic representation of the domain structure of the tetanus toxin precursor protein, showing the locations of the Heavy Chain and Light Chain and their respective functional roles. Also shown are the relative locations of the SEQ ID NO: 2 (TTC polypeptide) and SEQ ID NO: 5 (TTC polypeptide fragment) within the C-terminal region of the Heavy Chain of Tetanus Toxin.

FIG. 16 presents isometric muscle tension recording traces corresponding to twitch stimulus recording (left panel) and tetanic stimulus recording (right panel).

FIG. 17 presents quantitative analyses of muscle force in TA (FIG. 17A) and EDL (FIG. 17B) induced by twitch stimuli recorded at 120 days in SOD1G93A mice in controls (weekly pcDNA3.lEmpty and weekly TBS) and following TTC treatment (single dose pcDNA3.1 TCC, weekly dose pcDNA3.1TTC, and weekly TTC protein 10 micrograms). Error bars represent SEM; n=7-8 mice per group, two muscles recorded from each subject. *p<0.05 ; **p=<0.005.

FIG. 18 presents quantitative analyses of muscle force in TA (FIG. 18A) and EDL (FIG. 18B) induced by tetanic stimuli recorded at 120 days in SOD1G93A mice in controls (weekly pcDNA3.lEmpty and weekly TBS) and following TTC treatment (single dose pcDNA3.1 TCC, weekly dose pcDNA3.1TTC, and weekly TTC protein 10 micrograms). Error bars represent SEM; n=7-8 mice per group, two muscles recorded from each subject. *p<0.05; **p=<0.005.

FIG. 19 presents quantitative analyses of muscle contraction in TA (FIG. 19A) and EDL (FIG. 19B) induced by twitch stimuli recorded at 120 days in SOD1G93A mice in controls (weekly pcDNA3.lEmpty and weekly TBS) and following TTC treatment (single dose pcDNA3.1 TCC, weekly dose pcDNA3.1TTC, and weekly TTC protein 10 micrograms). Error bars represent SEM; n=7-8 mice per group, two muscles recorded from each subject. *p<0.05; **p=<0.005.

FIG. 20 presents quantitative analyses of muscle relaxation in TA (FIG. 20A) and EDL (FIG. 20B) induced by twitch stimuli recorded at 120 days in SOD1G93A mice in controls (weekly pcDNA3.lEmpty and weekly TBS) and following TTC treatment (single dose pcDNA3.1 TCC, weekly dose pcDNA3.1TTC, and weekly TTC protein 10 micrograms). Error bars represent SEM; n=7-8 mice per group, two muscles recorded from each subject. *p<0.05; **p=<0.005.

FIG. 21 presents quantitative analyses of normalized tetanic force:mass ratio in TA (FIG. 21A) and EDL (FIG. 21B) induced by tetanic stimuli recorded at 120 days in SOD1G93A mice in controls (weekly pcDNA3.lEmpty and weekly TBS) and following TTC treatment (single dose pcDNA3.1 TCC, weekly dose pcDNA3.1TTC, and weekly TTC protein 10 micrograms). Error bars represent SEM; n=7-8 mice per group, two muscles recorded from each subject. *p<0.05; **p=<0.005.

FIG. 22 presents quantitative analyses of muscle mass ratio in TA (FIG. 22A) and EDL (FIG. 22B) at 120 days in SOD mice in controls (weekly pcDNA3.1Empty and weekly TBS) and following TTC treatment (single dose pcDNA3.1 TCC, weekly dose pcDNA3.1TTC, and weekly TTC protein 10 micrograms). Error bars represent SEM; n=7-8 mice per group, two muscles recorded from each subject. *p<0.05; **p=<0.005.

FIG. 23 presents high magnification microscopy images showing muscle-specific effects following TTC protein treatment. Superficial triceps surae muscle from mice treated with vehicle (left) and TTC (right) are shown. Dashed squares indicate location of the region of interest shown at higher magnification in the lower panels. Scale bar as 100 μm.

FIG. 24 presents a genic biomarker assessment of 80 day old SOD1G93A mice on EDL muscle. The graph shows the fold change in transcriptional levels of the selected genes tested in EDL muscle of transgenic SOD1G93A mice at Day 80 with respect to the Wild Type. Error bars represent SEM; *p<0.05; **p=<0.01. wt: wild type mice, tg: not-treated SOD1G93A mice, ttc: SOD1G93A mice treated with two i.p. administrations of 10 μg/injection at day 60 and 75.

FIG. 25 presents a genic biomarker assessment of 80 day old SOD1G93A mice on SOLEUS muscle. The graphs shows the fold change in transcriptional levels of the selected genes tested in SOLEUS muscle of transgenic SOD1G93A mice at Day 80 with respect to the Wild Type. Error bars represent SEM; *p<0.05; **p=<0.01. wt: wild type mice, tg: not-treated SOD1G93A mice, ttc: SOD1G93A mice treated with two i.p. administrations of 10 μg/injection at day 60 and 75.

FIG. 26 shows the effect of intramuscular injection of TTC-protein on twitch (panel A) and tetanic forces (panel B) at 15 days following injury. Control: not-injured.

FIG. 27 shows a force-frequency curve of TA muscles in TTC-treated, PBS-treated and not injured (Control) groups (15 days). Data were expressed as mean±SEM.

FIG. 28 shows the effect of intramuscular injection of TTC-protein on twitch (panel A) and tetanic forces (panel B) at 30 days following injury

FIG. 29 shows a force-frequency curve of TA muscles in TTC- or PBS-treated groups (30 days). Data were expressed as mean±SEM

FIG. 30 shows the effect of intramuscular injection of TTC-plasmid on twitch (panel A) and tetanic forces (panel B) at 15 days following injury. Control: not-injured

FIG. 31 shows a force-frequency curve of TA muscles in TTC-, Empty-plasmid-treated and not injured (Control) groups (15 days). Data were expressed as mean±SEM.

FIG. 32 shows the effect of intramuscular injection of TTC-plasmid on twitch (panel A) and tetanic forces (panel B) at 30 days following injury.

FIG. 33 shows a force-frequency curve of TA muscles in TTC-plasmid- or Empty-plasmid-treated groups (30 days). Data were expressed as mean±SEM.

FIG. 34 shows the dose-response effect of TTC (1-100 nM) on C2C12 myoblast cell proliferation (48 hours, n=8). The mitogenic capacity was evaluated using BrdU Cell Proliferation ELISA Kit. Co: cells maintained 48 hours in DMEM. Control: cells in GM [DMEM+10% FBS (v/v); 48 hours]. Data were expressed as mean±SE, * P<0.05 versus control values.

FIG. 35 shows the dose-response effect of TTC (1-100 nM) on the protein expression of myogenin on differentiating C2C12 cells for 7 days (n=3). Levels of myogenin were represented as a fold of respective expression in GM (control). Data were expressed as the mean±SE obtained from intensity scans of independent experiments. *P<0.05 versus DM values.

FIG. 36 shows the dose-response effect of TTC (1-100 nM) on the protein expression of MHC on differentiating C2C12 cells for 7 days (n=3). Levels of MHC were represented as a fold of respective expression in GM (control). Data were expressed as the mean±SE obtained from intensity scans of independent experiments. *P<0.05 versus DM values.

FIG. 37 shows the dose-response effect of TTC (1-100 nM) on differentiating C2C12 cells for 7 days. Immunofluorescence detection of MHC and DAPI in C2C12 myotube cells under DM (control) or DM +TTC at the 7 day-point post-stimulation. Upper panels, objective magnification 10x. Lower panels, magnification of representative areas.

FIG. 38 shows the dose-response effect of TTC (1-100 nM) on the myotube area (μm²) at the 7-day point after stimulation (*, P<0.05).

FIG. 39 shows the dose-response effect of TTC (1-100 nM) on the myotube diameter (μm) at the 7-day point after stimulation (*, P<0.05).

FIG. 40 shows the dose-response effect of TTC (1-100 nM) on the fusion index at the 7-day point after stimulation (*, P<0.05).

FIG. 41 shows the dose-response effect of TTC (1-100 nM) on the number of myonuclei per MHC⁺ cell at the 7-day point after stimulation (*, P<0.05).

FIG. 42 shows the dose-response effect of TTC (1-100 nM) on the myotube orientation at the 7-day point after stimulation (*, P<0.05).

FIG. 43 shows immunofluorescence images illustrating the aggregated (panel A) or aligned (panel B) orientation of myotubes, and the dose-response effect of TTC (1-100 nM) on the nuclear distribution in C2C12 myotubes at the 7-day point after stimulation (*, P<0.05) (panel C).

### DETAILED DESCRIPTION

Tetanus toxin is a potent neurotoxin. Structurally, tetanus toxin (150 kDa) is comprised of two polypeptide chains, a heavy chain (100 kDa) and a light chain (50 kDa). One disulfide bridge connects these two polypeptides. The heavy chain contains the toxin's binding and translocation domains, whereas the light chain is a protease which cleaves synaptobrevin. The toxin first binds to gangliosides on peripheral nerve endings and is internalized through receptor-mediated endocytosis. The toxin then travels to the ventral horn by axoplasmic transport. From there it is released into the interneuronal space and is subsequently taken up by the inhibitory interneurons adjacent to the soma of the motor neurons.

An important fragment, the tetanus toxin C-fragment, is generated when the toxin is enzymatically cleaved by papain. This C-fragment (50 kDa) corresponds to the 451 amino acids at the C-terminus of the tetanus toxin heavy chain. The C-fragment is useful because it retains the binding, internalization and trans-synaptic transport capabilities of the whole toxin. However it is nontoxic since it does not disrupt any neuronal processes. The non-toxic carboxy-terminal domain of the heavy chain of the tetanus toxin (known in the art as TTC) has been used therapeutically as a carrier for therapeutic agents through the creation of fusion proteins which are retrogradely transported via nerves. See, e.g., Ciriza et al. Cent. Eur. J. Biol. 3: 105-112 (2008a); Ciriza et al. Restorative Neurology and Neuroscience 26: 459-65 (2008b).; Francis et al. Brain Res. 1011:7-13 (2004); U.S. Pat. Nos. 7,923,216, 7,972,608, 8,703,733, and 7,435,792; or Int'l Publ. Nos. WO2005000346, WO2011143557, and WO1999009057; all of which are herein incorporated by reference in their entireties. Furthermore, TTC has been recently proposed as a therapeutic agent to treat ALS due to its neuroprotective capacity. See Int'l. Publ. No. WO/2009/043963 and U.S. Pat. No. 8,945,586, which are herein incorporated by reference in their entireties.

The present disclosure provides evidence showing that administration of TTC (i.e., a TTC polypeptide, a polynucleotide encoding such TTC polypeptide, or a combination thereof) directly affects muscles, namely (i) increasing muscle mass in the subject, and/or (ii) increasing muscle strength in the subject, and/or (iii) increasing the rate of recovery or healing, and/or (iv) decreasing fibrosis caused by said disease or condition. The methods disclosed herein can also treat, prevent, reduce, or ameliorate the loss of muscle mass; decrease the rate of loss of muscle mass; treat, prevent, reduce, or ameliorate the symptoms associated with the loss of muscle mass; treat, prevent, reduce, or ameliorate the loss of muscle strength; treat, prevent, reduce or ameliorate the symptoms associated with the loss of muscle strength; treat, prevent, reduce, or ameliorate fibrosis caused by the disease or condition; or treat, prevent, reduce, or ameliorate the symptoms associated with the fibrosis caused by the disease or condition.

Thus, the present disclosure provides methods to (i) increase muscle mass in the subject, and/or (ii) increase muscle strength in the subject, and/or (iii) increase the rate of recovery or healing, and/or (iv) decrease fibrosis caused by said disease or condition in a subject in need thereof comprising the administration of TTC.

The present disclosure also provides methods to prevent or reduce loss of muscle mass and/or muscle strength. In addition, the present disclosure provides methods to treat the symptoms of a disease (e.g., a neuromuscular disease, HIV) or condition (e.g., aging, wound treatment for example after surgery, immobilization after fractures, muscle lesions, etc.) in which loss of muscle mass and/or muscle strength occurs, or to prevent or reduce loss of muscle mass and/or muscle strength in a subject in need thereof by administering TTC (i.e., a TTC polypeptide, a polynucleotide encoding such TTC polypeptide, or a combination thereof) to the subject. The administration of TTC can be for therapeutic uses and/or cosmetic uses related to increasing muscle mass and/or muscle strength, preventing loss of muscle mass and/or muscle strength, increasing the rate of recovery or healing, decreasing fibrosis caused by said disease or condition, and combinations thereof. In general, the methods disclosed herein can be applied whenever an increase in muscle mass or muscle strength is desirable (e.g., to counteract decrease of muscle mass due to aging or weightlessness, to improve the exercise capacity in normal healthy subjects, or to increase muscle mass in nonhuman animals).

The present disclosure also provides methods to monitor the effect of TTC on muscle (e.g., effects on muscle mass and/or muscle strength) comprising determining the level of at least one biomarker (e.g., Col19a1, Snx10, Calm1, Mef2c, or Col1A1) in a sample taken from a treated subject. In particular, the presence of levels of the biomarker above or below a predetermined threshold level can be used, e.g., (i) to determine whether a patient suffering a disease or condition in which loss of muscle mass and/or muscle strength occurs is eligible or non-eligible for a specific treatment with TTC (e.g., a TTC polypeptide, a TTC polynucleotide, or a combination thereof), (ii) to determine whether a specific treatment of a disease or condition in which loss of muscle mass and/or muscle strength occurs with TTC (e.g., a TTC polypeptide, a TTC polynucleotide, or a combination thereof) should commence, be suspended, or be modified, (iii) to diagnose whether a disease or condition in which loss of muscle mass and/or muscle strength occurs is treatable or not treatable with a specific TTC composition (e.g., a TTC polypeptide, a TTC polynucleotide, or a combination thereof), (iv) to prognosticate the outcome of treatment a disease or condition in which loss of muscle mass and/or muscle strength occurs with a with a specific TTC composition (e.g., a TTC polypeptide, a TTC polynucleotide, or a combination thereof).

In order that the present disclosure can be more readily understood, certain terms are first defined. Additional definitions are set forth throughout the detailed description.

### I. Definitions

In this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Wherever aspects are described herein with the language “comprising,” otherwise analogous aspects described in terms of “consisting of” and/or “consisting essentially of” are also provided.

The term “about” as used in connection with a numerical value throughout the specification and the claims denotes an interval of accuracy, familiar and acceptable to a person skilled in the art. In general, such interval of accuracy is ±15%.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component (e.g., a dye), a therapeutic agent (e.g., an agent to treat a neuromuscular disease), or a cosmetic agent. Also included within the definition are, for example, polypeptides containing one or more analogs of an amino acid (including, for example, unnatural amino acids, etc.), as well as other modifications known in the art.

A “recombinant” polypeptide or protein refers to a polypeptide or protein produced via recombinant DNA technology. Recombinantly produced polypeptides and proteins expressed in engineered host cells are considered isolated for the purpose of the invention, as are native or recombinant polypeptides which have been separated, fractionated, or partially or substantially purified by any suitable technique. The polypeptides disclosed herein can be recombinantly produced using methods known in the art. Alternatively, the proteins and peptides disclosed herein can be chemically synthesized.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. The preceding description applies to all polynucleotides referred to herein, including RNA and DNA.

The term “sequence” as used to refer to a protein sequence, a peptide sequence, a polypeptide sequence, or an amino acid sequence means a linear representation of the amino acid constituents in the polypeptide in an amino-terminal to carboxyl-terminal direction in which residues that neighbor each other in the representation are contiguous in the primary structure of the polypeptide.

A polypeptide, protein, inclusion body, or other composition disclosed herein which is “isolated” is a polypeptide, polynucleotide, vector, plasmid, or other composition disclosed herein which is in a form not found in nature. Isolated polypeptides, polynucleotides, vectors, plasmids, or other compositions disclosed herein include those which have been purified to a degree that they are no longer in a form in which they are found in nature. In some aspects, a polypeptide, polynucleotide, vector, plasmid, other composition disclosed herein as isolated is substantially pure.

The term “fragment” when referring to polypeptides or polynucleotides includes any polypeptides or polynucleotides which retain at least some of the properties of the reference polypeptides or polynucleotide. For example, in the case of TTC, the term fragment would refer for example to any polypeptide which retains at least to a certain degree a desirable property of the reference polypeptide, in particular the ability of the polypeptide to cause an increase in muscle mass and/or muscle strength when administered to a subject in need thereof. In the case of polynucleotides, the term fragment would refer to any polynucleotide which encodes a TTC polypeptide which retains the ability to cause an increase in muscle mass and/or muscle strength. Fragments of polypeptides include non-toxic proteolytic fragments (resulting from enzymatic or chemical proteolysis of tetanus toxin), deletion expression fragments (resulting for example from the expression of a fragment polynucleotide encoding a TTC fragment), and chemically synthesized fragment (for example, via solid phase peptide synthesis).

The term “variant” as used herein refers to a TTC sequence that differs from that of a parent sequence by virtue of at least one modification, e.g., a nucleotide modification or an amino acid modification. Variants can occur naturally or be non-naturally occurring. Non-naturally occurring variants can be produced using art-known mutagenesis techniques. Variant polypeptides can comprise conservative or non-conservative amino acid substitutions, deletions, or additions.

“Derivatives” of TTC polypeptides or polynucleotides which have been altered so as to exhibit additional features not found on the native polypeptide or polynucleotide. Also included as “derivatives” are those polypeptides that contain one or more naturally occurring amino acid derivatives of the twenty standard amino acids. A polypeptide or amino acid sequence “derived from” a designated polypeptide refers to the origin of the polypeptide.

Polypeptides derived from another peptide can have one or more mutations relative to the starting polypeptide, e.g., one or more amino acid residues which have been substituted with another amino acid residue or which has one or more amino acid residue insertions or deletions. In some aspects, the polypeptide comprises an amino acid sequence which is not naturally occurring. Such variants necessarily have less than 100% sequence identity or similarity with the starting polypeptide. In some aspects, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule. In one aspect, there is one amino acid difference between a starting polypeptide sequence and the sequence derived therefrom. Identity or similarity with respect to this sequence is defined herein as the percentage of amino acid residues in the candidate sequence that are identical (i.e. same residue) with the starting amino acid residues, after aligning the sequences and introducing gaps, if necessary, to achieve the maximum percent sequence identity.

Similarly, polynucleotides derived from another polynucleotide (e.g., a humanized TTC polynucleotide derived from the sequence of the wild type TTC polynucleotide or an optimized mRNA derived from a wild type or humanized RNA sequence encoding TTC) can have one or more mutations relative to the starting polynucleotide, e.g., one or more nucleotides or codons which have been substituted with another nucleotide or codon, or which has one or more nucleotide or codon insertions or deletions. In some aspects, the polynucleotide comprises a polynucleotide sequence which is not naturally occurring. Such variants necessarily have less than 100% sequence identity or similarity with the starting polynucleotide. In some aspects, the variant will have an nucleotide sequence from about 40% to less than 100% nucleotide sequence identity or similarity with the nucleotide sequence of the starting polynucleotide over the length of the variant molecule. In some aspects, the variant has about 40%, about 45%, about 50%, about 55%, about 60%, about 65%, about 70%, about 75%, about 80%, about 85%, about 90%, about 95%, about 96%, about 97%, about 98% or about 99% nucleotide sequence identity or similarity with the nucleotide sequence of the starting polynucleotide over the length of the variant molecule. In one aspect, there is one nucleotide difference between a starting polynucleotide sequence and the sequence derived therefrom. Identity or similarity with respect to this sequence is defined herein as the percentage of nucleotides in the candidate sequence that are identical (i.e. same nucleotide) with the starting nucleotides, after aligning the sequences and introducing gaps, if necessary, to achieve the maximum percent sequence identity.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

Non-conservative substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala, Ser) or no side chain (e.g., Gly).

Other substitutions can be readily identified by workers of ordinary skill. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, omithine, or D-ornithine. Generally, substitutions in functionally important regions that may be expected to induce changes in the properties of isolated polypeptides are those in which: (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions may alter functional properties of the protein is also correlated to the position of the substitution with respect to functionally important regions of the protein: some non-conservative substitutions may accordingly have little or no effect on biological properties.

The term “percent sequence identity” between two polypeptide or polynucleotide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences may be accomplished using readily available software both for online use and for download. Suitable software programs are available from various sources, and for alignment of both protein and nucleotide sequences. One suitable program to determine percent sequence identity is b12seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity may be curated either automatically or manually.

The term “pharmaceutical composition” as used herein refers to a preparation which is in such form as to permit the biological activity of the active ingredient (e.g., a TTC polypeptide, a TTC polynucleotide or a combination thereof) to be effective, and which contains no additional components which are unacceptably toxic to a subject to which the composition would be administered. Such composition can be sterile.

As used herein the terms “treat,” “treatment,” or “treatment of” refers to reducing the potential for a certain disease or disorder, reducing the occurrence of a certain disease or disorder, and/or a reduction in the severity of a certain disease or disorder, preferably, to an extent that the subject no longer suffers discomfort and/or altered function due to it. For example, treating can refer to the ability of a therapy (e.g., a TTC polypeptide, a TTC polynucleotide or a combination thereof) when administered to a subject, to prevent a certain disease or disorder from occurring and/or to cure or to alleviate a certain disease symptoms, signs, or causes. Treating also refers to mitigating or decreasing at least one clinical symptom and/or inhibition or delay in the progression of the condition and/or prevention or delay of the onset of a disease or illness (e.g., delay the onset of loss of muscle mas or muscle strength, or delay the onset of fibrosis caused by the disease or condition). Thus, the terms “treat,” “treating” or “treatment of” (or grammatically equivalent terms) refer to both prophylactic and therapeutic treatment regimes. In some aspects, such disease or disorder is a disease, condition, or disorder in which a loss of muscle mass and/or muscle strength occurs. Diseases and conditions that can be treated using the compositions and methods disclosed herein are described more in detail below.

The present disclosure provides methods and systems generally providing a therapeutic benefit. A therapeutic benefit is not necessarily a cure for a particular disease or disorder, but rather encompasses a result which most typically includes alleviation of the disease or disorder or increased survival, elimination of the disease or disorder, reduction of a symptom associated with the disease or disorder, prevention or alleviation of a secondary disease, disorder or condition resulting from the occurrence of a primary disease or disorder, and/or prevention of the disease or disorder.

The terms “subject” or “patient” as used herein refer to any subject, particularly a mammalian subject, for whom diagnosis, prognosis, or therapy of a disease or disorder is desired. As used herein, the terms “subject” or “patient” include any human or nonhuman animal. The term “nonhuman animal” includes all vertebrates, e.g., mammals and non-mammals, such as nonhuman primates, sheep, dogs, cats, horses, cows, bears, chickens, amphibians, reptiles, etc. As used herein, the term includes subjects, such as mammalian subjects, that would benefit from the administration of a therapy, imaging or other diagnostic procedure, and/or preventive treatment for a disease or disorder. In some aspects, TTC (e.g., a TTC polypeptide, a TTC polynucleotide or a combination thereof) can be used to increase muscle mass in an animal subject, for example, cattle. Unless otherwise indicated, the term “subject in need thereof” refers to a mammal, including but not limited to, a human exhibiting muscle loss and/or loss of muscle strength due at least in part to age, inactivity, disease or disorder, condition, or combinations thereof.

In some aspects of the present disclosure, a subject is a naïve subject. A naïve subject is a subject that has not been administered a therapy, for example a therapeutic agent to promote muscle growth and/or muscle strength and/or to prevent the loss of muscle mass. In some aspects, a naïve subject has not been treated with a therapeutic agent to promote muscle growth and/or muscle strength and/or prevent the loss of muscle mass, for example, a small molecule drug, prior to being administered TTC (e.g., a TTC polypeptide, a TTC polynucleotide or a combination thereof). In another aspect, a subject has received therapy and/or one or more doses of a therapeutic agent to promote muscle growth and/or muscle strength and/or prevent the loss of muscle mass, for example, a small molecule drug, prior to being administered TTC. In some aspects, a subject can receive a therapeutic agent to promote muscle growth and/or muscle strength and/or prevent the loss of muscle mass, e.g., a small molecule drug, concurrently with the administration of TTC.

The term “therapy” as used herein includes any means for curing, mitigating, or preventing a disease, condition, or disorder in which a loss of muscle mass and/or muscle strength occurs., including, for example, therapeutic agents, instrumentation, supportive measures, and surgical or rehabilitative procedures. In this respect, the term therapy encompasses any protocol, method and/or therapeutic or diagnostic that can be used in prevention, management, treatment, and/or amelioration of a disease, condition, or disorder in which a loss of muscle mass and/or muscle strength occurs.

As used herein the term “fibrosis” is to be understood as the formation or development of excess fibrous connective tissue in an organ or tissue as a reparative or reactive process, as opposed to a formation of fibrous tissue as a normal constituent of an organ or tissue.

The term “therapeutic agent” as used herein refers to any therapeutically active substance that is administered to a subject having a disease, condition, or disorder in which a loss of muscle mass and/or muscle strength occurs.to produce a desired, usually beneficial, effect. The term therapeutic agent includes, e.g., classical low molecular weight therapeutic agents commonly referred to as small molecule drugs and biologics including but not limited to: antibodies or active fragments thereof, peptides, lipids, protein drugs, protein conjugate drugs, enzymes, oligonucleotides, ribozymes, genetic material, prions, virus, bacteria, and eukaryotic cells. A therapeutic agent can also be a pro-drug, which metabolizes into the desired therapeutically active substance when administered to a subject. In some aspects, the therapeutic agent is a prophylactic agent. In addition, a therapeutic agent can be pharmaceutically formulated. A therapeutic agent can also be a radioactive isotope or agent activated by some other form of energy such as light or ultrasonic energy, or by other circulating molecules that can be systemically administered.

A “therapeutically effective” amount as used herein is an amount of therapeutic agent that provides some improvement or benefit to a subject having a disease, condition, or disorder in which a loss of muscle mass and/or muscle strength occurs. Thus, a “therapeutically effective” amount is an amount that provides some alleviation, mitigation, and/or decrease in at least one clinical symptom of the disease, condition, or disorder in which a loss of muscle mass and/or muscle strength occurs. Those skilled in the art will appreciate that therapeutic effects need not be complete or curative, as long as some benefit is provided to the subject.

In some aspects, the term “therapeutically effective” refers to an amount of a therapeutic agent therapeutic agent that is capable of (a) increasing muscle mass; (b) increasing muscle strength; (c) reducing loss of muscle mass caused by the disease or condition; (d) reducing loss of muscle strength caused by the disease or condition; (e) preventing the loss of muscle mass caused by the disease or condition; (f) preventing the loss of muscle strength caused by the disease or condition; (g) increasing the rate of recovery or healing from the disease or condition; (h) preventing fibrosis caused by the disease or condition; (i) decreasing fibrosis caused by the disease or condition; or, (j) a combination thereof.

As used herein, a “sufficient amount” or “an amount sufficient to” achieve a particular result in a patient having a disease, condition, or disorder in which a loss of muscle mass and/or muscle strength occurs refers to an amount of a therapeutic agent (e.g., a TTC polypeptide, a TTC polynucleotide, or a combination thereof) that is effective to produce a desired effect, which is optionally a therapeutic effect (i.e., by administration of a therapeutically effective amount). In some aspects, such particular result is:

(a) an increase in muscle mass;

(b) an increase in muscle strength;

(c) a reduction in loss of muscle mass caused by the disease or condition;

(d) a reduction in loss of muscle strength caused by the disease or condition;

(e) prevention of the loss of muscle mass caused by the disease or condition;

(f) prevention of the loss of muscle strength caused by the disease or condition;

(g) increase in the rate of recovery or healing from the disease or condition;

(h) prevention of fibrosis caused by the disease or condition;

(i) decrease in fibrosis caused by the disease or condition; or,

(j) a combination thereof.

The term “sample” as used herein includes any biological fluid or tissue, e.g., muscle tissue, obtained from a subject. Samples can be obtained by any means known in the art. In some aspects, a sample can be derived by taking biological samples from a number of subjects and pooling them or pooling an aliquot of each subjects' biological sample. The pooled sample can be treated as a sample from a single subject. The term sample also includes experimentally separated fractions of all of the preceding.

In some aspects, a sample can be a combination of samples from a subject, e.g., muscle tissue samples from different muscles. In some aspects, a sample can be a combination of samples from a population of subjects. In some aspects, multiple samples can be taken from a single subject at different time intervals, for example to monitor the progression of a disease, condition, or disorder in which a loss of muscle mass and/or muscle strength occurs, and/or to monitor the effect of treatment with TTC (e.g., a TTC polypeptide, a TTC polynucleotide or a combination thereof) or lack thereof when TTC is administered to a subject with a disease, condition, or disorder in which a loss of muscle mass and/or muscle strength occurs.

In order to apply the methods and systems of the disclosure, samples from a patient can be obtained before or after the administration of a therapy to treat a disease or disorder in which a loss of muscle mass and/or muscle strength occurs, for example, the administration of a TTC polypeptide, a TTC polynucleotide, or a combination thereof. In some cases, successive samples can be obtained from the patient after therapy has commenced or after therapy has ceased.

Samples can, for example, be requested by a healthcare provider (e.g., a doctor) or healthcare benefits provider, obtained and/or processed by the same or a different healthcare provider (e.g., a nurse, a hospital) or a clinical laboratory, and after processing, the results can be forwarded to the original healthcare provider or yet another healthcare provider, healthcare benefits provider or the patient. Similarly, the measuring/determination of one or more scores, comparisons between scores, evaluation of the scores and treatment decisions can be performed by one or more healthcare providers, healthcare benefits providers, and/or clinical laboratories.

The term “increased” with respect to a functional characteristic is used to indicate that the relevant functional characteristic is significantly increased relative to that of a reference, as determined under comparable conditions. In some aspects, the increase in the functional characteristic (e.g., increased muscle function, such as increase in overall muscle contractile force, twitch force, tetanic force, muscle mass, force:mass ratio, or a combination thereof) is, e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% higher relative to a reference, as determined under comparable conditions.

In some aspects, the increase in the functional characteristic (e.g., increased muscle function, such as increase in overall muscle contractile force, twitch force, tetanic force, muscle mass, force:mass ratio, or a combination thereof) is, e.g., an at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 3-fold, at least about 4-fold, at least about 5-fold, at least about 6-fold, at least about 7-fold, at least about 8-fold, at least about 9-fold, at least about 10-fold, increase relative to a reference, as determined under comparable conditions.

The term “decreased” with respect to a functional characteristic is used to indicate that the relevant functional characteristic is significantly decreased relative to that of a reference, as determined under comparable conditions. In some aspects, the decrease in the functional characteristic (e.g., decrease or prevent loss of muscle function and/or decrease or prevent loss of muscle mass) is, e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% lower relative to a reference, as determined under comparable conditions.

In some aspects, the decrease in the functional characteristic (e.g., decrease or prevent loss of muscle function or decrease or prevent loss of muscle mass) is, e.g., an at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 3-fold, at least about 4-fold, at least about 5-fold, at least about 6-fold, at least about 7-fold, at least about 8-fold, at least about 9-fold, at least about 10-fold, decrease relative to a reference, as determined under comparable conditions.

As used herein, the term “healthcare provider” refers to individuals or institutions that directly interact and administer TTC (e.g., a TTC polypeptide, a TTC polynucleotide or a combination thereof) to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “clinical laboratory” refers to a facility for the examination or processing of materials derived from a living subject, e.g., a human being, which is being treated, or may benefit from treatment of a disease, condition, or disorder in which a loss of muscle mass and/or muscle strength occurs with TTC (e.g., a TTC polypeptide, a TTC polynucleotide or a combination thereof).

Non-limiting examples of processing include biological, biochemical, serological, chemical, immunohematological, hematological, biophysical, cytological, pathological, genetic, or other examination of materials derived from the human body, e.g., muscle samples, for the purpose of providing information, e.g., for the diagnosis, prevention, or treatment of any disease or impairment of, or the assessment of the health of living subjects, e.g., human beings. These examinations can also include procedures to collect or otherwise obtain a sample, prepare, determine, measure, or otherwise describe the presence or absence of various substances in the body of a living subject, e.g., a human being, or a sample obtained from the body of a living subject, e.g., a human being.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

In some aspects, a healthcare provider can administer or instruct another healthcare provider to administer a therapy to treat a disease, condition, or disorder in which a loss of muscle mass and/or muscle strength occurs, for example, to administer TTC (e.g., a TTC polypeptide, a TTC polynucleotide, or a combination thereof).

A healthcare provider can implement or instruct another healthcare provider or patient to perform, for example, the following actions: obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapy, commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some aspects, a healthcare benefits provider can authorize or deny, for example, collection of a sample, processing of a sample, submission of a sample, receipt of a sample, transfer of a sample, analysis or measurement a sample, quantification a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent.

In addition a healthcare benefits provider can, e.g., authorize or deny the prescription of a therapy, authorize or deny coverage for therapy, authorize or deny reimbursement for the cost of therapy, determine or deny eligibility for therapy, etc.

In some aspects, a clinical laboratory can, for example, collect or obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, or other related activities.

### II. TTC Polypeptides and Polynucleotides

The term “TTC” as used herein refers to TTC polypeptides, TTC polynucleotides (i.e., polynucleotides that encode a TTC polypeptide and when expressed produce a TTC polypeptide), and/or combinations thereof. TTC polypeptides relate to the carboxyl-terminal portion of tetanus toxin, and in particular to the non-toxic tetanus toxin C-fragment generated when the toxin is enzymatically cleaved by papain. This C-fragment (50 kDa) corresponds to the 451 amino acids at the C-terminus of the tetanus toxin heavy chain, between amino acid positions 865 and 1315 (SEQ ID NO:5). Thus, in the context of the present disclosure the term TTC when referring to polypeptides corresponds to the fragment of tetanus resulting from digestion of the native protein with papain and equivalent fragments obtain via enzymatic digestion with other proteases, or through recombinant expression of the fragment, Recombinant expression of TTC is disclosed in U.S. Pat. No. 5,443,966, which is herein incorporated by reference in its entirety.

In some aspects, TTC, and in particular a TTC polypeptide, comprises the polypeptide sequence of SEQ ID NO: 2. In other aspects, TTC comprises the polypeptide sequence of SEQ ID NO: 5. In other aspects, TTC, and in particular a TTC polynucleotide, comprises the polynucleotide sequence of SEQ ID NO: 1, which encodes the polypeptide sequence of SEQ ID NO: 2. In other aspects, TTC comprises the polynucleotide sequence of SEQ ID NO: 6, which encodes the polypeptide sequence of SEQ ID NO: 5. SEQ ID NO:2 encompasses from the amino acid valine (V) at the amino terminal end of the tetanus toxin C-fragment generated when the toxin is enzymatically cleaved by papain to the amino acid aspartate (D) at the carboxy terminal of the tetanus toxin C-fragment generated when the toxin is enzymatically cleaved by papain, i.e., from V854 to D1315 of the tetanus protein sequence with NCBI Accession Number P04958. SEQ ID NO: 5 is a fragment of SEQ ID NO: 2 in which the N-terminal sequence VFSTPIPFSYS is absent.

The instant disclosure encompasses TTC polypeptides exhibiting a degree of sequence identify of at least about 70%, at least about 75%, at least about 80%, at least about 85%, at least about 90%, at least about 95%, or at least about 99% with the amino acid sequences presented herein as SEQ ID NO: 2 and SEQ ID NO: 5 (TTC fragment), as determined using any one of the programs described above.

Also enclosed in the definition of the term TTC are TTC polynucleotides capable of hybridizing under stringent conditions with the natural TTC sequence from the tetanus toxin gene. The stringent conditions are for example as follows: at 42° C. for 4 to 6 hours in the presence of 6×SSC buffer, 1× Denhardt's Solution, 1% SDS, and 250 μg/ml of tRNA. (1×SSC corresponds to 0.15 M NaCl and 0.05 M sodium citrate; 1× Denhardt's solution corresponds to 0.02% Ficoll, 0.02% polyvinyl pyrrolidone and 0.02% bovine serum albumin). The two wash steps are performed at room temperature in the presence of 0.1×SCC and 0.1% SDS.

In some aspects, the term TTC refers to the TTC gene, which includes genomic DNA, cDNA, mRNA, and fragments thereof. In some aspects, the TTC oligonucleotide comprises nucleobases different from A, T, C, G, or U, for example, universal bases. TTC polynucleotide variants can be created by recombinant techniques employing genomic or cDNA cloning methods. Site-specific and region-directed mutagenesis techniques can be employed. In addition, linkerscanning and PCR-mediated techniques can be employed for mutagenesis. See PCR TECHNOLOGY (Erlich ed., Stockton Press 1989). Protein sequencing, structure and modeling approaches for use with any of the above techniques are disclosed in See CURRENT PROTOCOLS IN MOLECULAR BIOLOGY vol. 1, ch. 8 (Ausubel et al. eds., J. Wiley & Sons 1989 & Supp. 1990-93); PROTEIN ENGINEERING (Oxender & Fox eds., A. Liss, Inc. 1987). If desired, other variations to TTC can be undertaken by employing combinatorial chemistry, biopanning and/or phage display. Peptide mimetics of TTC can be produced, for example, by the approach outlined in Saragovi et al, Science 253:792-95 (1991) and other articles. Mimetics are peptide-containing molecules which mimic elements of protein secondary structure. See, for example, Johnson et al, “Peptide Turn Mimetics” in BIOTECHNOLOGY AND PHARMACY, Pezzuto et al, Eds., (Chapman and Hall, New York, 1993).

In some aspects, the term TTC refers to a synthetic polynucleotide, e.g., a synthetic DNA or a synthetic RNA, such as a synthetic mRNA, encoding a TTC polypeptide or a fragment or variant thereof. The synthetic polynucleotide can comprise, for example, backbone modifications (e.g., phosphorothioate) and/or nucleotide analogues. In some aspects, nucleotide analogues are selected from the group consisting of a 2′-O-methoxyethyl-RNA (2′-MOE-RNA) monomer, a 2′-fluoro-DNA monomer, a 2′-O-alkyl-RNA monomer, a 2′-amino-DNA monomer, a locked nucleic acid (LNA) monomer, a cEt monomer, a cMOE monomer, a 5′-Me-LNA monomer, a 2′-(3-hydroxy)propyl-RNA monomer, an arabino nucleic acid (ANA) monomer, a 2′-fluoro-ANA monomer, an anhydrohexitol nucleic acid (HNA) monomer, an intercalating nucleic acid (INA) monomer, and a combination of two or more of said nucleotide analogues.

In some aspects, the synthetic mRNA polynucleotide is “sequence optimized” mRNA comprising, e.g., pseudouridine (Ψ), 5-methoyxuridine (5moU), 2-thiouridine (s2U), 4-thiouridine (s4U), N1-methylpseudouridine (1mΨ), or 5-methylcytidine, replacing one or more uridines and/or cytidines. In some aspects, synthetic mRNA sequences can be optimized by replacing, e.g., 25%, 50% or 100% of uridines with 4-thiouridine or 2-thiouridine (s2U). Exemplary RNA molecules encoding TTC with the sequences of SEQ ID NO: 9, SEQ ID NO:10 and SEQ ID NO:11 are disclosed herein.

The term TTC also includes fragments and variants (e.g., mutants comprising deletions, insertions, substitution, inversions, etc.) of a wild type TTC polypeptide or TTC polynucleotide, and derivatives thereof (e.g., glycosylated or aglycosilated protein forms of the TTC protein, or otherwise chemically modified forms of the protein; polynucleotides comprising nucleotide variants).

One skilled in the art would appreciate that the design of TTC fragments and variants to practice the methods disclosed herein can be guided by the considerable knowledge about the three-dimensional structure of TTC, which has been known since 1997 (see Umland et al., “Structure of the Receptor Binding Fragment Hc of Tetanus Toxin,” Nature Structural Biology 4:788-792). Additional crystal structures of TTC alone or as part of complexes have been produced since then (see, e.g., Fotinou et al. “The crystal structure of tetanus toxin Hc fragment complexed with a synthetic GT1b analogue suggests cross-linking between ganglioside receptors and the toxin,” Journal of Biological Chemistry 276: 32274-32281, 2001). Together with copious amounts of biochemical data, biophysical data, functional data, the available three-dimensional data provides considerable guidance for the design of fragments, variants, and derivatives conserving the properties of the parent TTC molecule. Such TTC fragments, variants, and derivatives could then be tested to verify that they are able to increase muscle mass and/or muscle strength and/or prevent muscle loss using method known in the art or the methods disclosed in the present disclosure.

The use of TTC polypeptides as carrier molecules for therapeutic agents, immunogens, or detectable moieties (e.g., GFP) is known in the art, as well as methods to link TTC to such molecules via genetic fusion or conjugation. For example, the generation of TTC derivatives via chemical conjugation has been disclosed in Dobrenis et al. Proc. Natl. Acad. Sci. 89:2297-2301 (1992); Francis et al. J. Biol. Chem. 270: 15434-15442 (1995); Knight et al. Eur. J. Biochem. 259:762-769 (1999); or Schneider et al. Gene Ther. 7: 1584-1592 (2000). Similarly, the generation of TTC derivatives through genetic fusion has been described, for example, in Coen et al. Proc. Natl. Acad. Sci. 94:9400-9405 (1997); Francis et al. J. Neurochem. 74:2528-2536 (2000); Matthews et al. J. Mol. Neurosci. 14: 155-166 (2000); and Kissa et al. Mol. Cell Neurosci., 20:627-637 (2002). The generation of humanized versions of TTC has been described in Intl. Publ. WO2011/143557 and U.S. Pat No. 8,703,733, which are herein incorporated by reference in their entireties.

As discussed above, TTC comprises amino acids 865 to 1315 of the tetanus toxin heavy chain. Mutant forms known in the art to show no differences in binding to neuronal membranes with respect to the wild type form of TTC comprise D1309A, F1305A, W1303A, R1168A, Y1170A, E1310Q, D1309N, E1310Q/D1309N, R1160K, N1292A, K1295A, and K1297A. See Sutton et al. FEBS Letter 493: 45-49 (2001). Mutant forms T1308A, D1309A, and E1310, as well as TTC fragments comprising deletions ΔV1306-D1315 or AG1311-D1315 have binding capabilities over 80% of the binding observed in the wild type form of TTC. The TTC deletion fragment ΔV1306-D1315 has been shown to be capable of binding to motorneurons and retrograde transport in addition to neuronal cell binding.

Amino acid residues 1274-1279 of TTC form a loop which joins two β sheets within the β-trefoil domain. This region is essential for biological activity because mutants lacking these residues exhibit greatly reduced binding to both gangliosides and neuronal cells and do not undergo retrograde transport.

A second loop joining also two β sheets within the β-trefoil domain is also essential for biological activity. Mutant proteins containing a deletion of six residues in this loop (ΔD1214-N1219) bind poorly to gangliosides and neuronal cells. See Sinha et al. Molecular Microbiology 37:1041-1051 (2000).

The term “TTC derivatives” includes conjugates (e.g., conjugates produced by chemical or enzymatic conjugation) and also includes chimeric polypeptides that may be produced by fusing a nucleic acid sequence (or a portion thereof) encoding a heterologous polypeptide to a nucleic acid sequence (or a portion thereof) encoding a TTC polypeptide. Techniques for producing chimeric polypeptides are standard techniques well known in the art. Such techniques usually require joining the sequences such that they are in the same reading frame, and expression of the fused polypeptide under the control of the same promoter(s) and terminator. In some aspects, TTC derivatives can be produced using chemical synthesis, i.e., nucleic acid synthesis or peptide synthesis.

“Heterologous polypeptide” as used herein refers to any non-TTC polypeptide sequence. Exemplary heterologous sequences include a heterologous signal sequence (e.g., native rat albumin signal sequence, a modified rat signal sequence, or a human growth hormone signal sequence) or a sequence used for purification of a TTC polypeptide (e.g., a histidine tag). The heterologous signal sequence peptides can be selected, for example, from the group consisting of a growth factor signal peptide, a hormone signal peptide, a cytokine signal peptide and an immunoglobulin signal peptide (IgSP). Thus, examples of signal peptides are signal peptides selected from the group consisting of TGFβ signal peptides, GDF signal peptides, IGF signal peptides, BMP signal peptides, neurotrophin signal peptides, PDGF signal peptide and EGF signal peptide, signal peptides selected from a hormone signal peptide, said hormone being selected from the group consisting of growth hormone, insulin, ADH, LH, FSH, ACTH, MSH, TSH, T3, T4, and DHEA, or an interleukin signal peptide. In one aspect, the signal peptide is selected from the group consisting of albumin signal peptide, modified albumin signal peptide, and growth hormone signal peptide, such as a signal peptide selected from the group consisting of rat albumin signal peptide, modified rat albumin signal peptide, and human growth hormone signal peptide, such as rat albumin signal peptide and human growth hormone signal peptide. In some aspects, TTC can be generically fused or genetically conjugated to a molecule that confers advantageous pharmacokinetic properties, for example, reduced clearance or extended plasma half-like, for example polyethylene glycol (PEG) or peptides such as HAP, PAS, XTEN, albumin, etc.

In some aspects, a TTC polynucleotide can be fused to additional polynucleotides, for example promoters, terminators, silencer sequences, sequences that facilitate its integration in chromosomes or any type of organizational structure of genetic material, etc. In other aspects, a TTC polynucleotide can be an mRNA comprising (i) at least one 5′ cap structure (e.g., Cap0, Cap1, ARCA, inosine, N1-methyl-guanosine, 2′-fluoro-guanosine, 7-deaza-guanosine, 8-oxo-guanosine, 2-amino-guanosine, LNA-guanosine, or 2-azido-guanosine); (ii) a 5′-UTR; and (iii) a 3′-UTR. In some aspects, the mRNA can also comprise a poly-A tail

In some aspects, TTC can be part of a vector. The term “vector” means a construct, which is capable of delivering, and in some aspects, expressing, one or more gene(s) or sequence(s) of interest in a host cell, e.g., an eukaryotic host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, yeast artificial chromosomes (YAC), bacterial artificial chromosomes (BAC), human artificial chromosomes (HAC), adenoviruses, retroviruses and any other type of DNA or RNA molecule capable of self-replication, and certain eukaryotic cells, such as producer cells.

As used herein, the term “vector” is intended to encompass a singular “vector” as well as plural “vectors. ” Vectors can be transfected so as to cause the cell, e.g., a muscle cell, to express a desired recombinant TTC polypeptide.

In some aspects, a TTC polypeptide (or vector comprising a TTC polynucleotide encoding it) can be used to generate a transgenic cell, e.g., a muscle cell, to express the desired recombinant polypeptide. Thus, in some case a TTC polynucleotide can be integrated in cell's a genome, e.g., in a chromosome, resulting in a cell that can express a TTC polypeptide. Such cell (e.g., an autologous cell or a heterologous cell) can then be transplanted to a subject suffering from a disease, condition, or disorder in which a loss of muscle mass and/or muscle strength occurs. The transfer of a TTC polynucleotide to a cell for such gene therapy approach can take place in vivo (e.g., by administering the TTC polynucleotide via an adenovirus) or ex vivo (e.g., by first extracting muscle cells from the subject and then transfecting them with a TTC polynucleotide).

Methods and vectors for genetically engineering cells and/or cell lines to express a polypeptide of interest are well known to those of skill in the art; for example, various techniques are illustrated in Current Protocols in Molecular Biology, Ausubel at al., eds. (Wiley & Sons, New York, 1988, and quarterly updates) and Sambrook et al., Molecular Cloning: A Laboratory Manual (Cold Spring Laboratory Press, 1989).

### III. Methods of Treatment with TTC

The present disclosure provides methods for treating diseases, conditions, or disorders associated with the loss of muscle mass and/or muscle strength comprising the administration of TTC (e.g., a TTC polypeptide, a TTC polynucleotide or a combination thereof), wherein the administration of TTC is effective in (a) increasing muscle mass; (b) increasing muscle strength; (c) reducing loss of muscle mass caused by the disease or condition; (d) reducing loss of muscle strength caused by the disease or condition; (e) preventing the loss of muscle mass caused by the disease or condition; (f) preventing the loss of muscle strength caused by the disease or condition; (g) increasing the rate of recovery or healing from the disease or condition; (h) preventing fibrosis caused by the disease or condition; (i) decreasing fibrosis caused by the disease or condition; or, (j) a combination thereof.

The present disclosure also provides compositions containing TTC (e.g., a TTC polypeptide, a TTC polynucleotide or a combination thereof) to treat diseases, conditions, or disorders associated with loss of muscle mass and/or muscle strength. Also provided is the use of TTC (e.g., a TTC polypeptide, a TTC polynucleotide or a combination thereof) in the preparation of a medicinal product for treating disease associates with the loss of muscle mass. Also provided is a composition containing TTC (e.g., a TTC polypeptide, a TTC polynucleotide or a combination thereof) which is effective in (a) increasing muscle mass; (b) increasing muscle strength; (c) reducing loss of muscle mass caused by the disease or condition; (d) reducing loss of muscle strength caused by the disease or condition; (e) preventing the loss of muscle mass caused by the disease or condition; (f) preventing the loss of muscle strength caused by the disease or condition; (g) increasing the rate of recovery or healing from the disease or condition; (h) preventing fibrosis caused by the disease or condition; (i) decreasing fibrosis caused by the disease or condition; or, (j) a combination thereof.

Also provided is the use of TTC (e.g., a TTC polypeptide, a TTC polynucleotide or a combination thereof) for (a) increasing muscle mass; (b) increasing muscle strength; (c) reducing loss of muscle mass caused by the disease or condition; (d) reducing loss of muscle strength caused by the disease or condition; (e) preventing the loss of muscle mass caused by the disease or condition; (f) preventing the loss of muscle strength caused by the disease or condition; (g) increasing the rate of recovery or healing from the disease or condition; (h) preventing fibrosis caused by the disease or condition; (i) decreasing fibrosis caused by the disease or condition; or, (j) a combinations thereof.

In some aspects, the subject does not suffer from a disease, condition, or disorder in which a loss of muscle mass and/or muscle strength occurs, but increase of muscle mass is desired (e.g., TTC can be used to increase muscle mass in an animal subject to increase meat production, or can be used in a human subject to increase muscle mass for cosmetic purposes).

The present invention also includes methods of treating conditions or afflictions which can be cured, alleviated or improved by (a) increasing muscle mass; (b) increasing muscle strength; (c) reducing loss of muscle mass caused by the disease or condition; (d) reducing loss of muscle strength caused by the disease or condition; (e) preventing the loss of muscle mass caused by the disease or condition; (f) preventing the loss of muscle strength caused by the disease or condition; (g) increasing the rate of recovery or healing from the disease or condition; (h) preventing fibrosis caused by the disease or condition; (i) decreasing fibrosis caused by the disease or condition; or, (j) combinations thereof, in a subject comprising the administration of TTC (e.g., a TTC polypeptide, a TTC polynucleotide or a combination thereof).

In general, the methods disclosed herein achieve the following desired effects: (i) increased muscle mass; (b) increased muscle strength; (c) reduced loss of muscle mass caused by the disease or condition; (d) reduced loss of muscle strength caused by the disease or condition; (e) prevention of the loss of muscle mass caused by the disease or condition; (f) prevention of the loss of muscle strength caused by the disease or condition; (g) increased rate of recovery or healing from the disease or condition; (h) prevention of fibrosis caused by the disease or condition; (i) decrease of fibrosis caused by the disease or condition; or, (j) a combination thereof, following the administration of one or more doses of TTC (e.g., a TTC polypeptide, a TTC polynucleotide or a combination thereof) to a subject in need thereof. In some aspects, the desired effect is muscle regeneration. In some aspects, the mechanisms by which these desired effects are achieved include (a) an increase in myogenesis, (b) an increase in myoblast proliferation, (c) an increase in myoblast differentiation, (d) an increase in myoblast size (e.g., myoblast area or myoblast diameter), or (f) a combination thereof. Accordingly, the present disclosure also provides methods to increase myogenesis, methods to increase myoblast proliferation, methods to increase myoblast differentiation, and methods to increase myoblast size (myotube hypertrophy) comprising administering TTC (e.g., a TTC polypeptide, a TTC polynucleotide or a combination thereof) to a subject in need thereof.

“Muscle” in the context of the present disclosure means preferably striated muscle tissue or muscle cells derived from striated muscle tissue such as skeletal muscle cells/tissue and cardiac muscle cells (cardiomyocytes) and cardiac muscle tissue.

Unless otherwise indicated, the term “muscle strength” refers to the amount of force a muscle, or muscle groups in sum, can exert either in acute tests of maximum force, or in time-dependent tests of muscle endurance, time dependent tests of muscle fatigue, or time dependent tests of muscle endurance and fatigue.

As used in the present disclosure, the term “muscle mass” encompasses both muscle weight and muscle volume. Unless otherwise indicated, the term “muscle function” refers to at least one of muscle mass, muscle strength, and muscle quality.

The term “muscle quality” as used herein refers to the amount of muscle strength per unit volume, cross-sectional area, or mass of the corresponding muscle, muscle groups, or arm or leg compartment, i.e., the term “muscle quality” refers to muscle strength per corresponding muscle volume, muscle strength per corresponding muscle cross-sectional area, or muscle strength per corresponding muscle mass. For example, leg muscle quality could refer, for example, to leg muscle strength/leg muscle volume or to leg muscle strength/leg muscle mass.

“Muscle wasting” as use herein refers to temporary or permanent loss of muscle mass. Diseases or conditions in which muscle wasting can occur (i.e., wasting disorders or conditions) include, for example, cachexia, anorexia, muscular dystrophy, neuromuscular disease, sequelae of immobilization, chronic disease, cancer, old age, or injury.

Various neuromuscular diseases are generally associated with loss of muscle mass. For example, myopathies, which involve damage to the actual muscle fibers, are an important group of these muscular diseases, and among them, progressive muscular dystrophies are characterized by a atrophy of the muscles, as well as abnormalities in the muscle biopsy showing modifications of the tissue. This group notably includes Duchenne muscular dystrophy (or DMD), Becker muscular dystrophy (or BMD) and the limb girdle muscular dystrophies. Other diseases and disorders in which loss of muscle mass has been observed include, without limitation, multi-infarct dementia, stroke, trauma, infections, meningitis, encephalitis, Pick's Disease, frontal lobe degeneration, corticobasal degeneration, multiple system atrophy, progressive supranuclear palsy, Creutzfeldt-Jakob disease, Lewy body disease, neuroinflammatory disease, spinal muscular atrophy, Parkinson's Disease, Alzheimer's Disease, amyotrophic lateral sclerosis, neuro AIDS, Chron's Disease, Huntington's Disease, gliomas, cancers (including brain metastasis), HIV-1 associated dementia (HAD), HIV associated neurocognitive disorders (HAND), paralysis, multiple sclerosis (MS), CNS-associated cardiovascular disease, prion disease, metabolic disorders, and lysosomal storage diseases (LSDs). Loss of muscle mass has also been observed in lysosomal storage diseases such as, without limitation, Gaucher's disease, Pompe disease, Niemann-Pick, Hunter syndrome (MPS II), Mucopolysaccharidosis I (MPS I), GM2-gangliosidoses, Gaucher disease, Sanfilippo syndrome (MPS IIIA), Tay-Sachs disease, Sandhoff s disease, Krabbe's disease, metachromatic leukodystrophy, and Fabry disease.

In addition, cachexia or marasmus is also a medical condition targeted by the methods and compositions disclosed herein. This state is characterized by extreme thinness, especially caused by muscle loss, caused by prolonged illness or inadequate calorie or protein intake. This condition is particularly seen in cases of chronic disease such as cancer or AIDS or in individuals with either heart failure, where there is atrophy of skeletal muscles in 60% of patients, or urinary incontinence. Although not actually considered as pathological, some situations are associated with loss of muscle mass, such as ageing, prolonged immobilization, etc. Here again, therefore, there is a reason for increasing the muscle mass. The methods of the present disclosure can also be used in increasing animal meat production, or in cosmetic applications where an increase in muscle mass and/or muscle strength and/or muscle function is desired.

The methods disclosed herein can be applied to treat a tissue wound in need of healing and/or accelerated healing. Unless specified, the term “wound” is used herein in its generic sense, meaning that it encompasses all types of wounds and injuries. The term “wound” encompasses burns, ulcers, lacerations, incisions, etc. “Wound” and “lesion” may be used interchangably herein, and unless the context specifically dictates otherwise, no distinction is intended. Lesions/wounds can be acute or chronic. Examples of acute wounds include, but are not limited to, surgical wounds (i.e., incisions), penetrating wounds, avulsion injuries, crushing injuries, shearing injuries, burn injuries, lacerations, and bite wounds. Examples of chronic wounds include, but are not limited to, ulcers, such as arterial ulcers, venous ulcers, pressure ulcers, and diabetic ulcers. Of course, acute wounds can become chronic wounds.

The TTC compositions disclosed herein (e.g., a TTC polypeptide, a TTC polynucleotide or a combination thereof) can be applied to an open wound or a closed wound. The compositions disclosed herein can be administered to any wound, anywhere it is desirable to promote wound healing. Accordingly, the compositions disclosed herein can be applied to increase the rate of recovery or healing. The compositions are also useful to reduce scarring after a wound is closed and/or healed.

Compositions to increase muscle mass and/or muscle strength and/or muscle function, such as TTC (e.g., a TTC polypeptide, a TTC polynucleotide or a combination thereof), can be used in settings in which patients have undergone surgery (or will undergo surgery), e.g., for joint replacement or repair, etc. As such, TTC compositions that are administered to promote the rescue of muscle mass would ideally not interfere with other aspects of surgical recovery such as wound healing.

The methods disclosed herein can be applied to treat a muscle lesion in need of healing and/or accelerated healing. The term “muscle lesion” as used herein refers to a bodily injury which disrupts the normal integrity of the tissue muscle structures and/or disrupts the normal function of the tissue muscle structures and/or causes a pathological change in a muscle. In some aspects, the muscle lesion can be acute or chronic. Functional muscle lesions generally do not show macroscopic evidence of muscle tear (measured, for example, by MRI or ultrasound). On the other hand, structural muscle lesions show macroscopic evidence of muscle tear.

The term muscle lesion encompasses mechanical lesions such as cuts, puncture injuries, bite wounds, gunshot wounds, abrasions, contusions, or lacerations. The term muscle lesion also includes thermal lesions caused by exposure to low temperatures (e.g., frostbite) or high temperatures (e.g., burns). Also encompassed by the term muscle lesion are chemical lesions caused, for example, by exposure to acid or alkali.

The term muscle lesion also includes iatrogenic muscle lesions. The term “iatrogenic muscle lesion” refers to a muscle lesion induced in a patient by a physician's or other medical caregiver's activity, manner, or therapy, e.g., a lesion that is either induced by, or results from a medical procedure (e.g., injection, incision, puncture, osteotomy, excision, etc.).

The term muscle lesion also encompasses muscle injuries related to repeated activities (for example, occupational or repeated stress injuries caused by, e.g., operating machinery or office equipment) and athletic muscle lesion (e.g., strains, muscle tears, or contusions). In some aspects, the term muscle lesion refers to athletic muscle injuries such as fatigue-induced muscle disorder (type 1A muscle injury), delayed-onset muscle soreness (DOMS) (type 1B muscle injury), spine-related neuromuscular muscle disorder (type 2A muscle injury), muscle-related neuromuscular muscle disorder (type 2B muscle injury), minor partial muscle tear (type 3A muscle injury), moderate partial muscle tear (type 3B muscle injury), (sub)total muscle tear/tendinous avulsion (type 4 muscle injury), or direct muscle injury (contusion). Some athletic muscle injuries encompassed in the definition of muscle lesion are also known popularly as “strains,” “pulled-muscles,” “hardening,” “hypertonus,” etc. See de Souza et al. (2013) Journal of Electromyography and Kinesology 23: 1253-1260; Mueller-Wollhfahrt et al. (2012) Br. J. Sports Med. 47(6):342-50, which are herein incorporated by reference in their entireties.

The TTC compositions disclosed herein (e.g., a TTC polypeptide, a TTC polynucleotide or a combination thereof) can be applied to treat a muscle lesion (e.g., a sports-related strain, a iatrogenic muscle lesion, a traumatic muscle lesion, etc). The compositions disclosed herein can be administered to any muscle lesion, anywhere it is desirable to heal the lesion or to accelerate its healing. Accordingly, the compositions disclosed herein can be applied to heal, and/or to increase the rate of recovery or healing of a muscle lesion. In some aspects, a muscle lesion can be treated by administering a therapeutically effective amount of TTC (e.g., a TTC polypeptide, a TTC polynucleotide or a combination thereof) directly at the site of the lesion or to a location in close proximity to the site of lesion, for example, by injection. In other aspects, the muscle lesion can be treated by administering the TTC at a distal location, e.g., by injection.

In some aspects, TTC administration comprises the administration of a TCC polypeptide (e.g., wild type TTC or a fragment, variant, or derivative thereof), a TTC polynucleotide (e.g., a wild type TTC, a humanized TTC, a sequence optimized TTC, or a fragment, variant, or derivative thereof), or a combination thereof. In some aspects, TTC is a polypeptide comprising the sequence of SEQ ID NO:2 or SEQ ID NO:5, or a fragment, variant, or derivative thereof. In some aspects, TTC is a polypeptide consisting of the sequence of SEQ ID NO:2 or SEQ ID NO:5. In some aspects, TTC is a polypeptide consisting essentially of the sequence of SEQ ID NO:2 or SEQ ID NO:5.

In other aspects, TTC is a polynucleotide comprising the sequence of SEQ ID NO:1 or SEQ ID NO:6, or a fragment, variant, or derivative thereof. In other aspects, TTC is a polynucleotide consisting of the sequence of SEQ ID NO:1 or SEQ ID NO:6. In other aspects, TTC is a polynucleotide consisting essentially of the sequence of SEQ ID NO:1 or SEQ ID NO:6. In some aspects, TTC is part of recombinant protein, a fusion protein, or a conjugate. In other aspects, TTC is part of nucleic acid encoding a recombinant protein or fusion protein.

In some aspects, TTC is a humanized polynucleotide comprising, e.g., the sequence of SEQ ID NO: 7 (a humanized TTC nucleotide sequence comprising two flanking sequences comprising Xho I sites to facilitate cloning) or the sequence of SEQ ID NO: 8, or a fragment, variant, or derivative thereof. In some aspects, TTC comprises, consists, or consists essentially of a polynucleotide consisting of the sequence of SEQ ID NO:8 or a fragment, variant, or derivative thereof.

In some aspects, TTC is an mRNA polynucleotide sequence comprising, e.g., the sequence of SEQ ID NOS:9, 10, or 11.

In some aspects, TTC comprises:

(a) a polypeptide comprising the sequence of SEQ ID NO:2 or SEQ ID NO:5, or a fragment, variant, or derivative thereof;

(b) a polypeptide consisting of the sequence of SEQ ID NO:2 or SEQ ID NO:5, or a fragment, variant, or derivative thereof;

(c) a polynucleotide comprising the sequence of SEQ ID NO:1 or SEQ ID NO:6, or a fragment, variant, or derivative thereof;

(d) a polynucleotide consisting of the sequence of SEQ ID NO:1 or SEQ ID NO:6, or a fragment, variant, or derivative thereof; or,

(e) combinations thereof.

In other aspects, TTC comprises:

(a) a fusion protein or conjugate wherein a TTC polypeptide is the only therapeutic moiety;

(b) a fusion protein, or conjugate comprising at least two therapeutic moieties, wherein a TTC polypeptide is one of the therapeutic moieties;

(c) a nucleic acid encoding a fusion protein wherein a TTC polypeptide is the only therapeutic moiety;

(d) a nucleic acid encoding a fusion protein comprising at least two therapeutic moieties, wherein a TTC polypeptide is one of the therapeutic moieties; or,

(e) a combination thereof.

In some aspects, TCC comprises a polynucleotide which is administered as a naked DNA. In some aspects, TCC can be administered orally, parenterally, intramuscularly, or nasally. In some aspects, a TTC polynucleotide or TTC polypeptide can be administered into a muscle. In particular aspects, such TTC polynucleotide can express a TTC polypeptide in vivo in said muscle. In some aspects, a TTC polynucleotide can be inserted into an expression vector. In some aspects, such vector is capable of in vivo expression. In some aspects, the vector can comprise a promoter capable of expressing the TTC polypeptide encoded by said vector. In some aspects, the expression vector is the pcDNA3.1 expression vector. In some specific aspects, the promoter is pCMV. In some aspects, the method is performed in vivo in a mammal subject. In some aspect, such mammal subject is human. In some aspects, such mammal subject is non-human In some aspects, the method comprises inserting a TTC polynucleotide in a suitable vector and transfecting a muscle cell so the muscle cell expresses the TTC polypeptide. In some aspects, cells are transiently transfected. In other aspects, cells are stably transfected. In some aspects, the transfected cells are autologous cells. In other aspects, the transfected cells are heterologous cells. In yet other aspects, the transfected cells are stem cells. In some aspects, transfection can be performed in vivo in the subject. In other aspects, transfection can be performed ex vivo.

The amount of TTC (e.g., a TTC polypeptide, a TTC polynucleotide or a combination thereof) that can be administered to the subject is, generally, a therapeutically effective amount. Such therapeutically effective amount of TTC can cause a detectable increase in one or more of the following parameters: body weight, muscle mass (e.g., tibialis anterior (TA) mass, gastrocnemius (GA) mass, quadriceps muscle mass, etc.), muscle strength/power, muscle function, or any combination thereof. For example, therapeutically effective amount of TTC (e.g., a TTC polypeptide, a TTC polynucleotide or a combination thereof) when administered to a subject in need thereof (e.g., a subject suffering from a disease, condition, or disorder in which a loss of muscle mass and/or muscle strength occurs) can cause an increase of any combination of the parameters described above, for example in the TA or GA, of at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% or more, compared to control treated subjects.

In some aspects, TTC (e.g., a TTC polypeptide, a TTC polynucleotide or a combination thereof) can be administered according to the methods disclosed here at a dose of about 0.04 mg/kg, when TCC is a TTC polypeptide; o about 1.22 mg/kg, when TCC is a TCC polynucleotide (e.g., TTC in plasmid form such as the pCMV-TTC plasmid disclosed in the Examples section). In some aspects, the TTC polypeptide can be administered at a dose of about 0.010 mg/kg, about 0.015 mg/kg, about 0.020 mg/kg, about 0.025 mg/kg, about 0.030 mg/kg, about 0.035 mg/kg, about 0.040 mg/kg, about 0.050 mg/kg, about 0.055 mg/kg, about 0.060 mg/kg, about 0.065 mg/kg, about 0.070 mg/kg, or about 0.075 mg/kg. In other aspects, the TTC polynucleotide (e.g., a plasmid comprising a polynucleotide sequence encoding a TTC polypeptide as disclosed en the Examples) can be administered at a dose of about 0.10 mg/kg, about 0.15 mg/kg, about 0.20 mg/kg, about 0.25 mg/kg, about 0.30 mg/kg, about 0.35 mg/kg, about 0.40 mg/kg, about 0.45 mg/kg, about 0.50 mg/kg, about 0.55 mg/kg, about 0.60 mg/kg, about 0.65 mg/kg, about 0.70 mg/kg, about 0.75 mg/kg, about 0.80 mg/kg, about 0.85 mg/kg, about 0.90 mg/kg, about 0.95 mg/kg, about 1.00 mg/kg, about 1.05 mg/kg, about 1.10 mg/kg, about 1.15 mg/kg, about 1.20 mg/kg, about 1.25 mg/kg, about 1.30 mg/kg, about 1.35 mg/kg, about 1.40 mg/kg, about 1.45 mg/kg, about 1.50 mg/kg, about 1.55 mg/kg, about 1.60 mg/kg, about 1.65 mg/kg, about 1.70 mg/kg, about 1.75 mg/kg, about 1.80 mg/kg, about 1.85 mg/kg, about 1.90 mg/kg, about 1.95 mg/kg, about 2.00 mg/kg, about 2.05 mg/kg, about 2.10 mg/kg, about 2.15 mg/kg, about 2.20 mg/kg, about 2.20 mg/kg, about 2.25 mg/kg, about 2.30 mg/kg, about 2.35 mg/kg, about 2.40 mg/kg, about 2.45 mg/kg, or about 2.50 mg/kg.

In some aspects, TTC (e.g., a TTC polypeptide, a TTC polynucleotide or a combination thereof) can be administered at a fixed dose. In other aspects, TTC (e.g., a TTC polypeptide, a TTC polynucleotide or a combination thereof) can be administered as a variable dose. In some aspects, TTC can be administered as a single dose. In other aspects, TTC can be administered in multiple doses, for example two or more doses administered daily, weekly, biweekly, or monthly.

TTC (e.g., a TTC polypeptide, a TTC polynucleotide or a combination thereof) can be administered according to the methods of the instant disclosure along with one or more additional therapeutic agents, including, e.g., growth factor inhibitors, immunosuppressants, anti-inflammatory agents, metabolic inhibitors, enzyme inhibitors, and cytotoxic/cytostatic agents. The additional therapeutic agent(s) may be administered prior to, concurrent with, or after the administration of TTC.

### IV. Biomarkers to Detect TTC Effect

The present disclosure also provides biomarker to evaluate, for example, the effect of TTC (e.g., a TTC polypeptide, a TTC polynucleotide or a combination thereof), to determine whether a subject is a candidate to treatment with TTC, to monitor the progression of a disease or disorder prior, during, of after treatment with TCC. In some aspects, the methods disclosed herein require the measurement of the level of a biomarker selected from the group consisting of Col19a1 (Collagen alpha-1(XIX) chain; UniProtKB: Q14993), Snx10 (Sorting Nexin 10; UniProtKB: Q9Y5X0), Calml (Calmodulin 1 (Phosphorylase Kinase, Delta); UniProtKB: P62158), Mef2C (Myocyte Enhancer Factor 2C; UniProtKB: Q06413), and Col1A1 (Collagen, Type I, Alpha 1; UniProtKB: P02452).

The term “level of a biomarker” refers to a measurement that is made using any analytical method for detecting presence or expression of a biomarker (protein expression or gene expression) disclosed herein (e.g., Col19a1, Snx10, Calm1, Mef2c, or Col1A1), for example in a biological sample and that indicates the presence, absence, absolute amount or concentration, relative amount or concentration, titer, expression level, ratio of measured levels, or the like, of, for, or corresponding to the biomarker in the biological sample. The exact nature of the “value” or “level” depends on the specific designs and components of the particular analytical method employed to detect the biomarker (e.g., immunoassays, mass spectrometry methods, in vivo molecular imaging, gene expression profiling, aptamer-based assays, etc.).

As used herein with reference to the biomarkers disclosed herein (e.g., Col19a1, Snx10, Calm1, Mef2c, or Col1A1), the terms “elevated,” “elevated level,” or “high level” refer to a level in a biological sample (e.g., a muscle tissue sample) that is higher than a normal level or range. The normal level or range for a biomarker disclosed herein (e.g., Col19a1, Snx10, Calm1, Mef2c, or Col1A1) is defined in accordance with standard practice. Thus, the level measured in a particular biological sample can be compared with level or range of levels determined in similar normal samples. In this context, a normal sample would be a sample obtained from an individual who has not undergone treatment with TTC (e.g., a TTC polypeptide, a TTC polynucleotide or a combination thereof). The level of biomarker is said to be elevated wherein the biomarker is present in the test sample at a higher level or range than in a normal sample.

Biomarker levels (either expressed protein levels, or nucleic acid levels such as mRNA levels) can be detected and quantified by any of a number of methods well known to those of skill in the art. These methods include analytic biochemical methods such as electrophoresis, capillary electrophoresis, high performance liquid chromatography (HPLC), thin layer chromatography (TLC), hyperdiffusion chromatography, mass spectroscopy and the like, or various immunological methods such as fluid or gel precipitin reactions, immunodiffusion (single or double), immunohistochemistry, affinity chromatography, immunoelectrophoresis, radioimmunoassay (RIA), enzyme-linked immunosorbent assays (ELISAs), immunofluorescent assays, Western blotting, and the like.

In one aspect, the biomarker can be detected and/or quantified in an electrophoretic polypeptide separation (e.g., a 1- or 2-dimensional electrophoresis). Means of detecting polypeptides using electrophoretic techniques are well known to those skilled in the art (see generally, R. Scopes (1982) Polypeptide Purification, Springer-Verlag, N.Y.; Deutscher, (1990) Methods in Enzymology Vol. 182: Guide to Polypeptide Purification, Academic Press, Inc., N.Y.). A variation of this aspect utilizes a Western blot (immunoblot) analysis to detect and quantify the presence of the biomarker in the sample. This technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the sample with antibodies that specifically bind the analyte. Antibodies that specifically bind to the analyte may be directly labeled or alternatively may be detected subsequently using labeled antibodies (e.g., labeled sheep anti-mouse antibodies) that specifically bind to a domain of the primary antibody.

In some aspects, the sample and/or biomarker is transformed in some manner in the course of the detection and/or quantitation assay. For example, the sample can be fractionated such that biomarker is separated from at least one other sample component. Inn some aspects, a biomarker can be recovered in a liquid fraction or can be detected while embedded in a separation medium, such as a gel.

In a specific aspect, the biomarker is detected and/or quantified in the biological sample using an immunoassay. For a general review of immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991). In some aspects, the immunoassay can use one or more antibodies or antigen binding fragments thereof which recognize a specific biomarker.

In certain aspects, the immunoassay comprises a sandwich immunoassay, e.g., an enzyme-linked immunosorbent assay (ELISA) or a sandwich electrochemiluminescent (ECL) assay, in which a first “capture” antibody or antigen-binding fragment thereof is attached to a solid support, antigen from a sample or standard is allowed to bind to the capture antibody, and then a second “detection” antibody or antigen binding fragment thereof is added and detected either by an enzymatic reaction, an ECL reaction, radioactivity, or other detection method.

Based on comparison to known control samples, a “biomarker threshold level” can be determined, and test samples that fall above that biomarker threshold level (e.g., a Snx10 protein expression and/or gene expression threshold level) can indicate that the patient from whom the sample of taken may benefit from treatment with TTC (e.g., a TTC polypeptide, a TTC polynucleotide or a combination thereof). Biomarker threshold levels (e.g., protein expression levels or gene expression levels) must be predetermined, and must be matched as to the type of sample, the type of disease, and in some instances, the assay used. For example, threshold levels for Col10a1 or Snx10 can be determined from the experimental data provided in the present disclosure.

In particular aspects, the methods disclosed herein include informing the subject of a result of the biomarker assay and/or of a diagnosis based at least in part on the biomarker level (e.g., the level of Col19a1, Snx10, Calm1, Mef2c, Col1A1, or any combination thereof). The patient can be informed verbally, in writing, and/or electronically. This diagnosis can also be recorded in a patient medical record.

The methods disclosed herein also include prescribing, initiating, and/or altering prophylaxis and/or therapy, e.g., for a disease or disorder in which loss of muscle mass and/or loss of muscle strength occurs. In certain aspects, the methods can entail ordering and/or performing one or more additional assays.

### V. Methods of Diagnosis and Treatment Using Biomarkers

The present disclosure provides a method of treating a patient having a disease or condition associated with loss of muscle mass and/or loss of muscle strength comprising administering a therapeutically effective amount of TTC (e.g., a TTC polypeptide, a TTC polynucleotide or a combination thereof) to the subject if the level of Col19a1 (Collagen alpha-1(XIX) chain) and/or Snx10 (Sorting Nexin 10) in a sample taken from the patient is above a predetermined Col19a1 and/or Snx10 threshold level, or is above the Col19a1 and/or Snx10 level in one or more control samples, wherein said administration is effective to (i) increase muscle mass, and/or (ii) increase muscle strength, and/or (iii) increase the rate of recovery or healing, and/or (iv) decrease fibrosis caused by said disease or condition in the subject.

Also provided is method of treating a patient having a disease or condition associated with loss of muscle mass and/or loss of muscle strength comprising (a) submitting a sample taken from the patient for measurement of the level of Col19a1 and/or Snx10, and (b) administering a therapeutically effective amount of TTC (e.g., a TTC polypeptide, a TTC polynucleotide or a combination thereof) to the subject if the level of Col19a1 and/or Snx10 in the sample taken from the patient is above a predetermined Col19a1 and/or Snx10 threshold level, or is above the Col19a1 and/or Snx10 level in one or more control samples, wherein said administration is effective to (i) increase muscle mass, and/or (ii) increase muscle strength, and/or (iii) increase the rate of recovery or healing, and/or (iv) decrease fibrosis caused by said disease or condition in the subject.

The present disclosure also provides a method of treating a patient having a disease or condition associated with loss of muscle mass and/or loss of muscle strength comprising (a) measuring the level of Col19a1 and/or Snx10 submitting a sample taken from the patient, (b) determining whether the patient's level of Col19a1 and/or Snx10 is above a predetermined Col19a1 and/or Snx10 threshold level, or is above the Col19a1 and/or Snx10 level in one or more control samples, and (c) advising a healthcare provider to administer a therapeutically effective amount of TTC (e.g., a TTC polypeptide, a TTC polynucleotide or a combination thereof) to the subject, wherein said administration is effective to (i) increase muscle mass, and/or (ii) increase muscle strength, and/or (iii) increase the rate of recovery or healing, and/or (iv) decrease fibrosis caused by said disease or condition in the subject.

Also provided is a method of determining whether to treat a patient diagnosed with a disease or condition associated with loss of muscle mass and/or loss of muscle strength comprising (a) measuring, or instructing a clinical laboratory to measure the level of Col19a1 and/or Snx10 in a sample obtained from the patient; and (b) treating, or instructing a healthcare provider to treat, the patient by administering TTC (e.g., a TTC polypeptide, a TTC polynucleotide or a combination thereof) if the patient's level of Col19a1 and/or Snx10 in the sample is above a predetermined Col19a1 and/or Snx10 threshold level, or is above the level of Col19a1 and/or Snx10 in one or more control samples, wherein said administration is effective to (i) increase muscle mass, and/or (ii) increase muscle strength, and/or (iii) increase the rate of recovery or healing, and/or (iv) decrease fibrosis caused by said disease or condition in the subject.

Also provided is method of selecting a patient diagnosed with a disease or condition associated with loss of muscle mass and/or loss of muscle strength as a candidate for treatment with a TTC therapeutic regimen comprising (a) measuring, or instructing a clinical laboratory to measure the level of Col19a1 and/or Snx10 in a sample obtained from the patient; and (b) treating, or instructing a healthcare provider to treat the patient by administering TTC (e.g., a TTC polypeptide, a TTC polynucleotide or a combination thereof) if the patient's level of Col19a1 and/or Snx10 in the sample is above a predetermined Col19a1 and/or Snx10 threshold level, or is above the level of Col19a1 and/or Snx10 in one or more control samples, wherein said administration is effective to (i) increase muscle mass, and/or (ii) increase muscle strength, and/or (iii) increase the rate of recovery or healing, and/or (iv) decrease fibrosis caused by said disease or condition in the subject.

In some aspects, the diagnosis and treatment methods using biomarkers disclosed above also comprise determining the level of at least an additional biomarker, for example, Calml (Calmodulin 1 (Phosphorylase Kinase, Delta); UniProtKB: P62158), Mef2C (Myocyte Enhancer Factor 2C; UniProtKB: Q06413), or Col1A1 (Collagen, Type I, Alpha 1; UniProtKB: P02452).

In some aspects, the patient's biomarker level (e.g., the level of Col19a1, Snx10, Calm1, Mef2c, Col1A1, or any combination thereof) can be measured in an immunoassay employing one or more antibodies or antigen binding fragments thereof which recognize a certain biomarker. In other aspects, the patient's biomarker level (e.g., DNA and/or RNA level) is measured in an assay employing one or more oligonucleotide probes capable of specifically hybridizing to a certain biomarker gene.

In certain aspects, the detection assay (e.g., an immunoassay) can be performed on a sample obtained from the patient (e.g., a muscle tissue sample), by the healthcare professional treating the patient (e.g., using an immunoassay as described herein, formulated as a “point of care” diagnostic kit). In some aspects, a sample is obtained from the patient and is submitted, e.g., to a clinical laboratory, for measurement of the biomarker level in the sample according to the healthcare professional's instructions (e.g., using an immunoassay as described herein). In certain aspects, the clinical laboratory performing the assay will advise the healthcare provide as to whether the patient can benefit from treatment with TTC (e.g., a TTC polypeptide, a TTC polynucleotide or a combination thereof) based on whether the patient's biomarker level is above a predetermined biomarker threshold value or is elevated relative to one or more control samples.

In certain aspects of all method of treatment aspects provided herein, a “loading” dose of TTC (e.g., a TTC polypeptide, a TTC polynucleotide or a combination thereof) is administered to achieve a desired therapeutic level in the patient. If the loading dose does not affect the patient's biomarker levels (e.g., protein expression levels or gene expression levels) significantly or the patient's biomarker levels decrease, a decision could be made to discontinue treatment. If the loading dose results in steady or increased biomarker levels in the patient a decision could be made to reduce the dose size or frequency to a “maintenance” dose. It is important to note that the methods provided here are guidelines for a healthcare provider to administer treatment, and the ultimate treatment decision will be based on the healthcare provider's sound judgment.

In certain aspects, results of an immunoassay as provided herein can be submitted to a healthcare benefits provider for determination of whether the patient's insurance will cover treatment with TTC (e.g., a TTC polypeptide, a TTC polynucleotide or a combination thereof).

Similarly, this disclosure provides a method of monitoring the therapeutic efficacy of a TTC therapeutic regimen in a subject comprising: measuring, or instructing a clinical laboratory to measure the biomarker level (e.g., protein expression level or gene expression level) in a first sample obtained from the patient; administering, or advising a healthcare professional to administer TTC (e.g., a TTC polypeptide, a TTC polynucleotide or a combination thereof) to the patient if the patient's biomarker level (e.g., the level of Col19a1, Snx10, Calm1, Mef2c, Col1A1, or any combination thereof) in the first sample is below a predetermined threshold biomarker level, or is elevated relative to the biomarker level (e.g., the level of Col19a1, Snx10, Calm1, Mef2c, Col1A1, or any combination thereof) in one or more control samples; measuring the biomarker level in a second sample obtained from the patient, wherein the patient's biomarker level is again measured, and determining, or obtaining results indicating whether the patient's biomarker level in the second sample is higher than, about the same as, or lower than the biomarker level measured in the first sample; wherein the TTC therapeutic regimen is effective if the patient's biomarker level in the second sample is higher than or about the same as the biomarker level in the first sample.

In some aspects, the threshold is about a 1-fold, about a 2-fold, about a 3-fold, about a 4-fold, about a 5-fold, about a 6-fold, about a 7-fold, about an 8-fold, about 9-fold, or about a 10-fold increase in protein or gene expression with respect to control conditions. In some aspects, the threshold is about a 1-fold, about a 2-fold, about a 3-fold, about a 4-fold, about a 5-fold, about a 6-fold, about a 7-fold, about an 8-fold, about 9-fold, or about a 10-fold decrease in protein or gene expression with respect to control conditions.

In some aspects, the threshold is about a 1-fold, about a 2-fold, about a 3-fold, about a 4-fold, about a 5-fold, about a 6-fold, about a 7-fold, about an 8-fold, about 9-fold, or about a 10-fold increase in protein or gene expression with respect to the median value of a population of patients. In some aspects, the threshold is about a 1-fold, about a 2-fold, about a 3-fold, about a 4-fold, about a 5-fold, about a 6-fold, about a 7-fold, about an 8-fold, about 9-fold, or about a 10-fold decrease in protein or gene expression with respect to the median value of a population of patients.

In certain aspects, the one or more control samples used to identify the patient as a candidate for treatment with TTC (e.g., a TTC polypeptide, a TTC polynucleotide or a combination thereof) are obtained from normal healthy individuals. In other aspects, the one or more control samples used to identify the patient as a candidate for treatment with TTC (e.g., a TTC polypeptide, a TTC polynucleotide or a combination thereof) are obtained from the mean value of a population of subjects having the same disease or condition as the patient.

### VI. Pharmaceutical Compositions

The present disclosure provides formulations comprising TTC (e.g., a TTC polypeptide, a TTC polynucleotide or a combination thereof) formulated together with a diluent, carrier, or excipient. The present disclosure also provides pharmaceutical compositions comprising TTC (e.g., a TTC polypeptide, a TTC polynucleotide or a combination thereof) formulated together with a pharmaceutically acceptable diluent, carrier, or excipient. Such formulations or pharmaceutical compositions can include one or a combination of, for example, but not limited to, two or more different TTC compounds, e.g., a TTC polypeptide and a TTC polynucleotide. For example, a formulation or pharmaceutical composition disclosed herein can comprise a combination of TTC compounds that have different mechanisms of action (e.g., a TTC polypeptide which would be effective immediately after administration and a TTC polynucleotide that would have to be expressed in situ), or that have complementary activities (e.g., a TTC polypeptide with a short plasma half-life, and a long-acting TTC polypeptide conjugate).

To prepare pharmaceutical or sterile compositions including TTC, TTC can be mixed with a pharmaceutically acceptable carrier or excipient. Formulations of therapeutic and diagnostic agents can be prepared by mixing with physiologically acceptable carriers, excipients, or stabilizers in the form of, e.g., lyophilized powders, slurries, aqueous solutions, lotions, or suspensions.

Pharmaceutical compositions comprising TTC (e.g., a TTC polypeptide, a TTC polynucleotide or a combination thereof) also can be administered in combination therapy, such as, combined with other agents. For example, the combination therapy can include a TTC combined with at least one other therapy where the therapy can be surgery, immunotherapy, chemotherapy, radiation treatment, or drug therapy.

The pharmaceutical compounds can include one or more pharmaceutically acceptable salt. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, procaine, diethanolamine, ethylenediamine, and the like.

A pharmaceutical composition also can include a pharmaceutically acceptable anti-oxidant. Examples of pharmaceutically acceptable antioxidants include: (i) water soluble antioxidants, such as ascorbic acid, cysteine hydrochloride, sodium bisulfate, sodium metabisulfite, sodium sulfite and the like; (ii) oil soluble antioxidants, such as ascorbyl palmitate, butylated hydroxyanisole (BHA), butylated hydroxytoluene (BHT), lecithin, propyl gallate, alpha-tocopherol, and the like; and (iii) metal chelating agents, such as citric acid, ethylenediamine tetraacetic acid (EDTA), sorbitol, tartaric acid, phosphoric acid, and the like.

Examples of suitable aqueous and non-aqueous carriers that can be employed in the pharmaceutical compositions disclosed herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These pharmaceutical compositions can also contain adjuvants such as preservatives, wetting agents, emulsifying agents and dispersing agents. Prevention of presence of microorganisms can be ensured both by sterilization procedures and by the inclusion of various antibacterial and antifungal agents, for example, paraben, chlorobutanol, phenol, sorbic acid, and the like. It can also be desirable to include isotonic agents, such as sugars, sodium chloride, and the like into the compositions. In addition, prolonged absorption of the injectable pharmaceutical form can be brought about by the inclusion of agents that delay absorption such as aluminum monostearate and gelatin.

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it can be suitable to include isotonic agents, for example, sugars, poly-alcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, appropriate methods of preparation include vacuum drying and freeze-drying (lyophilization) that yield a powder of the active ingredient plus any additional desired ingredient from a previously sterile-filtered solution thereof.

In one aspect, the compositions herein are pyrogen-free formulations that are substantially free of endotoxins and/or related pyrogenic substances. Endotoxins include toxins that are confined inside a microorganism and are released when the microorganisms are broken down or die. Pyrogenic substances also include fever inducing, thermostable substances (glycoproteins) from the outer membrane of bacteria and other microorganisms. Both of these substances can cause fever, hypotension and shock if administered to humans. Due to the potential harmful effects, even low amounts of endotoxins can be appropriately removed from intravenously administered pharmaceutical drug solutions. The Food & Drug Administration (“FDA”) has set an upper limit of 5 endotoxin units (EU) per dose per kilogram body weight in a single one-hour period for intravenous drug applications. When therapeutic proteins are administered in amounts of several hundred or thousand milligrams per kilogram body weight even trace amounts of endotoxin may appropriately be removed.

In an aspect, endotoxin and pyrogen levels in the composition are less than 10 EU/mg, less than 5 EU/mg, less than 1 EU/mg, less than 0.1 EU/mg, less than 0.01 EU/mg, or less than 0.001 EU/mg. In certain embodiments, endotoxin and pyrogen levels in the composition are less than about 10 EU/mg, less than about 5 EU/mg, less than about 1 EU/mg, or less than about 0.1 EU/mg, less than about 0.01 EU/mg, or less than about 0.001 EU/mg.

Various delivery systems are known and can be used to administer the pharmaceutical compositions of the present disclosure, e.g., encapsulation in liposomes, microparticles, microcapsules, recombinant cells capable of expressing TTC, receptor mediated endocytosis (see, e.g., Wu et al., 1987, J. Biol. Chem. 262:4429-4432), etc. Methods of administration include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.), to name only a few.

In certain situations, the pharmaceutical compositions of the present disclosure can be delivered in a controlled release system. In one embodiment, a pump may be used (see Langer, supra: Sefton, 1987, CRC Crit. Ref. Biomed. Eng. 14:201). In another embodiment, polymeric materials can be used see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

### VII. Articles of Manufacture and Kits

The disclosure also provides articles of manufacture comprising any one of the compositions disclosed herein, e.g., TTC polypeptides, TTC polynucleotides, and combinations thereof, and pharmaceutical compositions comprising TTC, in one or more containers. In some aspect, the article of manufacture comprises, for example, a brochure, printed instructions, a label, or package insert directing the user (e.g., a distributor or the final user) to combine and/or use the compositions of the article of manufacture to promote muscle growth and/increase muscle strength and/or prevent loss of muscle mass and/or prevent loss of muscle strength in a subject in need thereof. In some aspect, the article of manufacture comprises, for example, a brochure, printed instructions, a label, or package insert directing the user (e.g., a distributor or the final user) to combine and/or use the compositions of the article of manufacture for cosmetic purposes or to promote muscle growth in an animal.

In some aspects, the article of manufacture comprises, for example, bottle(s), vial(s), cartridge(s), box(es), syringe(s), injector(s), or any combination thereof. In some aspects, the label refers to use or administration of the compositions (e.g., TTC polypeptides, TTC polynucleotides, and combinations thereof, and pharmaceutical compositions comprising TTC) in the article of manufacture according to the methods disclosed herein. In some aspects, the label suggests, for example, a regimen for use, a regimen for treating, preventing, or ameliorating a disease, condition, or disorder in which loss of muscle mass and/or muscle strength occurs.

This disclosure also provides kits for detecting the effectiveness of TTC (e.g., to determine the protein expression level or gene expression level on one or more biomarkers), for example, through an immunoassay method or nucleic acid detection method. Such kits can comprise containers, each with one or more of the various reagents (e.g., in concentrated form) utilized in the method, including, for example, one or more antibodies capable to specifically binding to at least one biomarker (e.g., Col19a1, Snx10, Calm1, Mef2c, Col1A1, or any combination thereof), or nucleic acid probes capable of specifically hybridizing to cDNA or mRNA for at least one biomarker. One or more antibodies against at least one biomarker, e.g., capture antibodies, or oligonucleotide probes can be provided already attached to a solid support. One or more antibodies against at least one biomarker, e.g., detection antibodies, or oligonucleotide probes can be provided already conjugated to a detectable label, e.g., biotin or a ruthenium chelate.

The kit can also provide reagents and instrumentation to support the practice of the assays provided herein. In certain aspects, a labeled secondary antibody can be provided that binds to the detection antibody. A kit provided according to this disclosure can further comprise suitable containers, plates, and any other reagents or materials necessary to practice the assays provided herein.

In some aspects, a kit comprises one or more nucleic acid probes (e.g., oligonucleotides comprising naturally occurring and/or chemically modified nucleotide units) capable of hybridizing a subsequence of a biomarker (e.g., a nucleic acid encoding all or part of Col19a1, Snx10, Calm1, Mef2c, Col1A1, or any combination thereof) with high stringency conditions. In some aspects, one or more nucleic acid probes (e.g., oligonucleotides comprising naturally occurring and/or chemically modified nucleotide units) capable of hybridizing a subsequence of a biomarker under high stringency conditions are attached to a microarray chip.

A kit provided according to this disclosure can also comprise brochures or instructions describing the process. Test kits can include instructions for carrying out one or more biomarker detection assays, e.g., immunoassays or nucleic acid detection assays. Instructions included in the kits can be affixed to packaging material or can be included as a package insert. While the instructions are typically written or printed materials they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### VIII. Embodiments

E1. A method of treating a disease or condition associated with decreased muscle mass and/or muscle strength in a subject in need thereof comprising administering a therapeutically effective amount of TTC to the subject, wherein said administration is effective to increase muscle mass and/or muscle strength and/or increase the rate of recovery or healing, and/or decrease fibrosis caused by said disease or condition in the subject.

E2. The method according to embodiment E1, wherein the disease or condition is a wasting disorder.

E3. The method according to embodiments E2, wherein the wasting disorder is selected from the group consisting of cachexia and anorexia.

E4. The method according to embodiments E2, wherein the wasting disorder is selected from the group consisting of a muscular dystrophy and a neuromuscular disease.

E5. The method according to embodiments E1 wherein the condition is a sequelae of immobilization, chronic disease, cancer, or injury.

E6. A method of increasing muscle mass in a subject in need thereof comprising administering TTC to the subject.

E7. The method according to embodiments E6, wherein the increase in muscle mass is to compensate for wasting resulting from a wasting disorder, immobilization, or old age.

E8. The method according to embodiments E6, wherein the increase in muscle mass is for cosmetic purposes.

E9. A method of treating a patient having a disease or condition associated with loss of muscle mass and/or loss of muscle strength comprising administering a therapeutically effective amount of TTC to the subject if the level of Col19a1 (Collagen alpha-1(XIX) chain) and/or Snx10 (Sorting Nexin 10) in a sample taken from the patient is above a predetermined Col19a1 and/or Snx10 threshold level, or is above the Col19a1 and/or Snx10 level in one or more control samples, wherein said administration is effective to (i) increase muscle mass, and/or (ii) increase muscle strength, and/or (iii) increase the rate of recovery or healing, and/or (iv) decrease fibrosis caused by said disease or condition in the subject.

E10. A method of treating a patient having a disease or condition associated with loss of muscle mass and/or loss of muscle strength comprising (a) submitting a sample taken from the patient for measurement of the level of Col19a1 and/or Snx10, and (b) administering a therapeutically effective amount of TTC to the subject if the level of Col19a1 and/or Snx10 in the sample taken from the patient is above a predetermined Col19a1 and/or Snx10 threshold level, or is above the Col19a1 and/or Snx10 level in one or more control samples, wherein said administration is effective to (i) increase muscle mass, and/or (ii) increase muscle strength, and/or (iii) increase the rate of recovery or healing, and/or (iv) decrease fibrosis caused by said disease or condition in the subject.

E11. A method of treating a patient having a disease or condition associated with loss of muscle mass and/or loss of muscle strength comprising (a) measuring the level of Col19a1 and/or Snx10 submitting a sample taken from the patient, (b) determining whether the patient's level of Col19a1 and/or Snx10 is above a predetermined Col19a1 and/or Snx10 threshold level, or is above the Col19a1 and/or Snx10 level in one or more control samples, and (c) advising a healthcare provider to administer a therapeutically effective amount of TTC to the subject, wherein said administration is effective to (i) increase muscle mass, and/or (ii) increase muscle strength, and/or (iii) increase the rate of recovery or healing, and/or (iv) decrease fibrosis caused by said disease or condition in the subject.

E12. A method of determining whether to treat a patient diagnosed with a disease or condition associated with loss of muscle mass and/or loss of muscle strength comprising (a) measuring, or instructing a clinical laboratory to measure the level of Col19a1 and/or Snx10 in a sample obtained from the patient; and (b) treating, or instructing a healthcare provider to treat, the patient by administering TTC if the patient's level of Col19a1 and/or Snx10 in the sample is above a predetermined Col19a1 and/or Snx10 threshold level, or is above the level of Col19a1 and/or Snx10 in one or more control samples, wherein said administration is effective to (i) increase muscle mass, and/or (ii) increase muscle strength, and/or (iii) increase the rate of recovery or healing, and/or (iv) decrease fibrosis caused by said disease or condition in the subject.

E13. A method of selecting a patient diagnosed with a disease or condition associated with loss of muscle mass and/or loss of muscle strength as a candidate for treatment with a TTC therapeutic regimen comprising (a) measuring, or instructing a clinical laboratory to measure the level of Col19a1 and/or Snx10 in a sample obtained from the patient; and (b) treating, or instructing a healthcare provider to treat the patient by administering TTC if the patient's level of Col19a1 and/or Snx10 in the sample is above a predetermined Col19a1 and/or Snx10 threshold level, or is above the level of Col19a1 and/or Snx10 in one or more control samples, wherein said administration is effective to (i) increase muscle mass, and/or (ii) increase muscle strength, and/or (iii) increase the rate of recovery or healing, and/or (iv) decrease fibrosis caused by said disease or condition in the subject.

E14. The method according to any one of embodiments E9 to E13, wherein the sample taken from the patient comprises muscle tissue.

E15. The method according to any one of embodiments E1 to E14, wherein the subject is human.

E16. The method according to any of embodiments E1 to E15, wherein TTC comprises:

(a) a polypeptide comprising the sequence of SEQ ID NO:2 or SEQ ID NO:5, or a fragment, variant, or derivative thereof;

(b) a polynucleotide comprising the sequence of SEQ ID NO:1 or SEQ ID NO:6, or a fragment, variant, or derivative thereof; or,

(c) combinations thereof.

E17. The method according to any one of embodiments E1 to E16, wherein TTC comprises:

(a) a fusion protein or conjugate wherein a TTC polypeptide is the only therapeutic moiety;

(b) a fusion protein, or conjugate comprising at least two therapeutic moieties, wherein a TTC polypeptide is one of the therapeutic moieties;

(c) a nucleic acid encoding a fusion protein wherein a TTC polypeptide is the only therapeutic moiety;

(d) a nucleic acid encoding a fusion protein comprising at least two therapeutic moieties, wherein a TTC polypeptide is one of the therapeutic moieties; or,

(e) a combination thereof.

E18. The method according to any one of embodiments E1 to E17, wherein TTC is administered as a naked DNA.

E19. The method according to any one of embodiments E1 to E8, wherein TTC is administered at a fixed dose.

E20. The method according to any one embodiments E1 to E19, wherein TTC is administered in two or more doses.

E21. The method according to any one of embodiments E1 to E20, wherein TTC is administered daily, weekly, biweekly, or monthly.

E22. The method according to any one of embodiments E1 to E21, wherein TTC is administered intramuscularly, intraperitoneally, subcutaneously, intravenously, or a combination thereof

E23. The method according to any one of embodiments E1 or E22, wherein said method is performed in vivo in a mammal.

E24. The method according to any one of embodiments E1 to E23, further comprising at least one additional therapy.

All patents and publications referred to in the present disclosure are expressly incorporated by reference in their entireties.

Aspects of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, can be practiced without departing from the scope of the present disclosure.

### EXAMPLES

### Example 1

### Administration of TTC by Intramuscular Injection of Naked DNA

The generation of transgenic animals that over-express the human gene for Superoxide Dismutase-1 (SOD-1) with different mutations has provided animal models for the study of ALS, a disease characterized among other symptoms, by a reduction in muscle function. These model animals present the same clinical and pathological characteristics as ALS patients.

### Materials and Methods

### 1.1 Naked DNA Encoding TTC

The gene encoding TTC (C-terminal domain of the heavy chain of the Tetanus toxin—SEQ ID NO: 2 of 462 amino acids-) was cloned in the eukaryote expression plasmid pcDNA3.1 {Invitrogen), under the control of the promoter of the cytomegalovirus (CMV). The vectors ware produced in chemically competent Escherichia coli bacteria (DH5α) and were purified using the GenElute maxiprep kit of Sigma-Aldrich.

### 1.2 Transgenic Mice

SOD1-G93A transgenic mice, which overexpress human SOD1 with the mutation G93A (B6SJL-TgN[SOD1-G93A]1Gur), were obtained from The Jackson Laboratory (Bar Harbor, Me.). Hemizygote mutants were used in all experiments (a mutant male mated with a non-transgenic female). The transgenic mice were identified by PCR amplification of the DNA extracted from the tail, as described in Gurney et al. Science 264: 1772-5 (1994). The animals were kept in the Mixed Research Unit of Zaragoza University. They were given food and water ad libitum. All experiments and care of the animals were conducted in compliance with the rules of Zaragoza University and of the international guide for the care and use of laboratory animals.

### 1.3 Intramuscular Injection of Naked DNA and Muscle Extraction

At 8 weeks of age the transgenic SOD1G93A mice were given intramuscular injections of 300 μg of pCMV-TTC in the quadriceps muscles (two injections of 50 μg per muscle) and in the triceps muscles (a single injection of 50 μg per muscle). The control group of mice was injected with the same amounts of empty plasmid. Ten days after the intramuscular injections of the plasmids, the inoculated muscles were extracted, pre-frozen in liquid nitrogen and subsequently stored at −70° C.

1.4 Extraction of RNA, Synthesis of cDNA and Amplification by PCR

To identify the presence of the transcribed TCC product in muscle, muscle tissue samples were frozen in liquid nitrogen and then pulverized in a cold mortar and pestle. The muscles total RNA was extracted following the TRIzol Reagent protocol (Invitrogen). For the synthesis of cDNA the kit SuperScript™ First-Strand Synthesis System (Invitrogen) was used, starting out with 1 μg of RNA in a final volume of 20 μL. The PCR reactions were carried out in a final volume of 20 μL, with 150 nM of each primer, 150 μM of dNTPs, 2 mM of MgCl₂ 1× buffer, 0.2U Taq pol and 2 μL per reaction of cDNA diluted 10 times for the amplification of a fragment of the TTC gene. All the PCR reactions were carried out in GeneAmp® Thermal Cycler 2720 (Applied Biosystems, Foster City, Calif., USA). The thermal cycle parameters were as follows: incubation at 94° C. during 3 minutes and 35 cycles of 94° C. during 30 seconds, 61° C. during 30 seconds and 72° C. during 30 seconds. The presence of the amplification of the TTC gene was observed in an agarose gel at 2% stained with ethidium bromide. The sequences of the used direct and reverse primers were SEQ ID NO: 3 and SEQ ID NO: 4, respectively. The size of amplification corresponds to 355 bp.

### 1.5 Rotarod, Grid Test

The animals carried out this test once a week from the age of 8 weeks. Each mouse was placed on a grid that serves as a lid for conventional cages. The grid was then turned 180° upside down and held at a distance of approximately 60 cm from a soft surface to avoid injury. The latency to fall of each mouse was timed. Each mouse had up to three attempts to hold onto the inverted grid for a maximum of 180 s and the longest period of time was recorded.

The Rotarod test was used to evaluate motor coordination and balance. The animals were placed on the rotating rod of the device (ROTAROD/RS, LE8200, LSI-LETICA Scientific Instruments). The time during which an animal could maintain itself on said bar at a constant speed of 14 rpm was recorded. Each mouse had three chances and the longest period of time without the animals falling from the bar was recorded, taking 180 s arbitrarily as the time limit. The end point in the life of the mice was considered to be when the animal was placed in supine position and was incapable of turning itself around.

### Results

### 2.1 Detection of the Expression of the TTC Plasmid in the Muscle

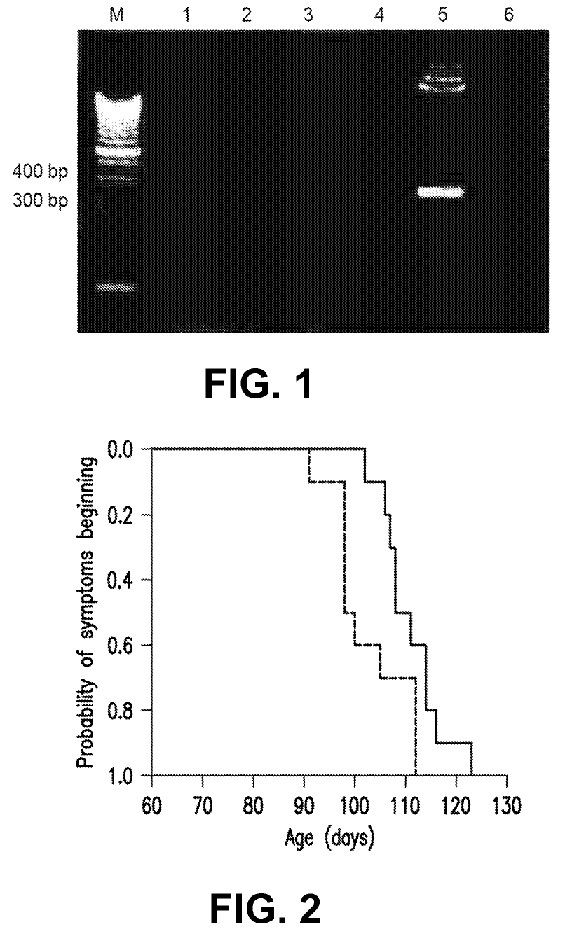

The capacity of the constructed vector pCMV-TTC to express the encoding gene in the muscular cell of the transgenic SOD1G93A mice was confirmed. Because there is no endogenous expression of the TTC gene in these mice, PCR amplification of a fragment of this gene was applied to the injected muscles in order to detect the expression of the mRNA of said molecule. As shown in FIG. 1, no expression of the TTC gene is observed in the control group injected with empty plasmid. However, the PCR reveals the presence of the amplification of the TTC gene in the muscle inoculated with the vector encoding same, indicating that the vector successfully reaches the muscular cells and that the process of transcription of said gene is carried out.

### 2.2 Effect of TTC in Transgenic SOD1G93A Mice

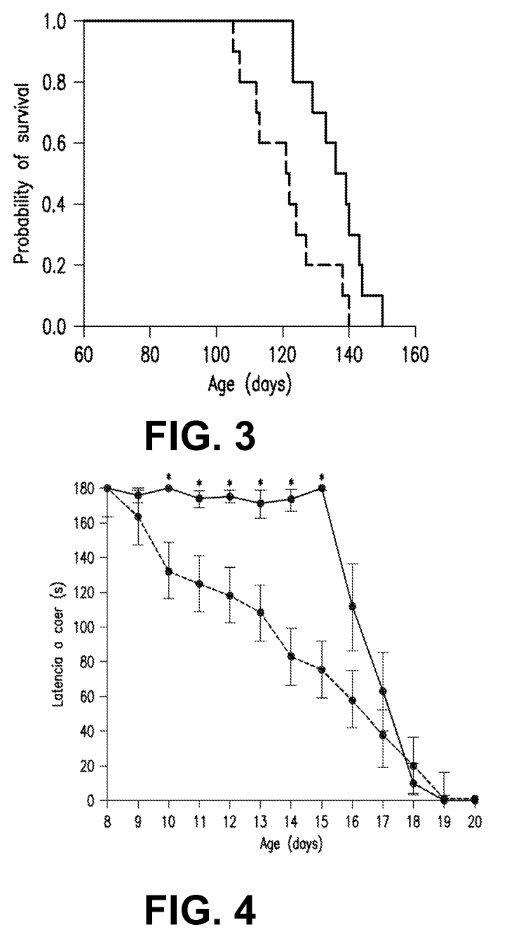

Intramuscular treatment with naked DNA encoding TCC delays the start of neuromuscular symptoms, and increases survival in the model mouse. The manifestation of symptoms was recorded as the first day on which the mice were unable to keep hold of the inverted grid for 3 minutes. The start of symptoms was reduced very significantly by approximately 8 days in the group of animals injected with TTC, in relation to the control group (FIG. 2, and TABLE 1). As shown in FIG. 3 and TABLE 1, maximum survival was detected in the group of mice treated with TTC, which reached an average of 136 days; 16 days more than the control group. Between weeks 12 and 13 a notable decrease was observed in the development of the Rotarod activity of the control group, whereas in the group of treated animals these deficiencies were not observed until week 16 (FIG. 4).

TABLE 1
Manifestation of symptoms (loss in muscle function)
and survival of both the control group and the group treated with TTC

| | | | P Value |
|---|---|---|---|
| | Control | TTC | (Log Rank, |
| | (n = 10) | (n = 10) | Mantel Cox) |
| Start of symptoms (days) | 102.4 ± 2.4 | 110.9 ± 2.0 | 0.0295 |
| Mortality (days) | 120.5 ± 3.9 | 136.0 ± 3 | 0.0093 |
| Difference in start − | 18.1 | 25.1 | |
| mortality (days) | | | |

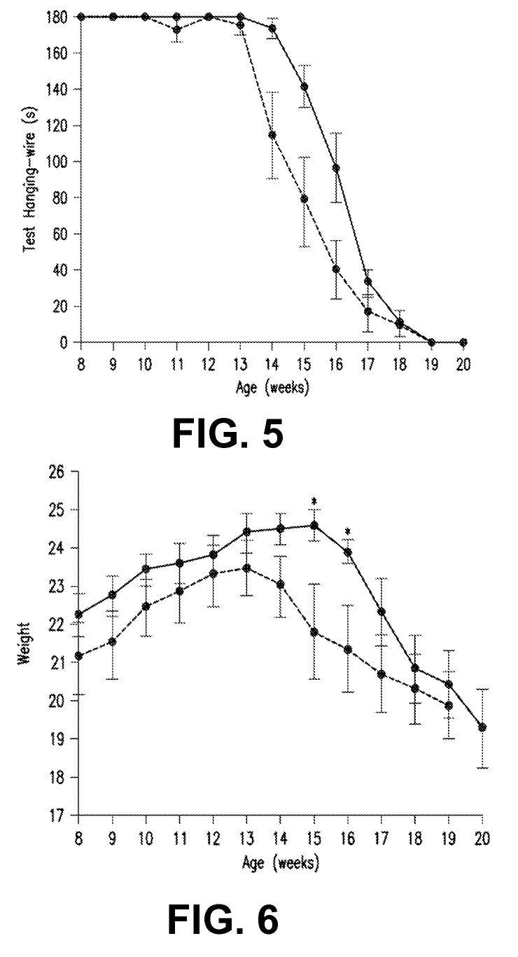

The treatment was also evaluated in mice starting at 8 weeks of age using the “hanging-wire” test (FIG. 5), another test to monitor muscle function. At 14 weeks of age, the SOD1G93A mice showed the first signs of weakness, whereas the group of mice treated with TTC proved to be more resistant between weeks 14-16. Also, the mice of the control group started to lose weight as of 14 weeks of age associated to the disease. However, the treatment with TTC significantly counteracted the weight loss, showing a maximum weight at 15 weeks (FIG. 6).

### Example 2

### Inhibition of Apoptosis in the Spinal Cord by Injection of Naked DNA Encoding TTC

### Materials and Methods

### 1.1 Naked DNA Encoding TTC

The gene encoding TTC (C-terminal domain of the heavy chain of the Tetanus toxin, SEQ ID NO: 1) was cloned in the eukaryote expression plasmid pcDNA3.1 (Invitrogen), under the control of the promoter of the cytomegalovirus (CMV). The vectors were produced in chemically competent Escherichia coli bacteria (DH5α) and were purified using the Genelute maxiprep kit of Sigma-Aldrich.

### 1.2 Transgenic Mice

The transgenic mice that overexpress human SOD1 with the mutation G93A (B6SJL-TgN[SOD1-G93A]1Gur) were obtained from The Jackson Laboratory (Bar Harbor, ME). Hemizygote mutants were used in all experiments (a mutant male mated with a non-transgenic female). The transgenic mice were identified by PCR amplification of the DNA extracted from the tail, as described in Gurney et al. (1994). The animals were kept in the Mixed Research Unit of Zaragoza University. They were given food and water ad libitum. All experiments and care of the animals were conducted in compliance with the rules of Zaragoza University and of the international guide for the care and use of laboratory animals. A total of 12 animals were used: wild-type (n=5), SOD1G93A mice injected with pcDNA3.1 (control, n=5) and SOD1G93A mice treated with TTC (n=5).

### 1.3 Intramuscular Injection of Naked DNA and Spinal Cord Extraction

At 8 weeks of age the transgenic SOD1G93A mice were given intramuscular injections of 300 μg of pCMV-TTC in the quadriceps muscles (two injections of 50 μg per muscle) and in the triceps muscles (one single injection of 50 μg per muscle). The control group of mice was injected with the same amounts of empty plasmid,

The spinal cords were extracted 110 days after the intramuscular injections of the plasmids. pre-frozen in liquid nitrogen and subsequently stored at −70° C. The tissues were frozen in liquid nitrogen and then pulverized in a cold mortar and pestle. Half of the sample was used for RNA extraction and the other half was used for protein extraction.

1.4 RNA Extraction from the Spinal Cord and Synthesis of cDNA

Spinal cord total RNA was extracted following the RNeasy® Lipid Tissue Mini Kit protocol (Qiagen). For the synthesis of cDNA the SuperScript™ First-Strand Synthesis System kit (Invitrogen) was used, starting out with 20 μg of RNA in a final volume of 20 μL

### 1.5 Real Time PCR

The real time PCR reactions were carried out in a final volume of 10 μL. with IX TaqMan® Universal PCR Master Mix. No AmpErase® UNG (Applied Biosystems). 1X the mixture of unmarked primers and TaqMan® MGB probes (Applied Biosystems) for each gene under study and 1 μL per reaction of cDNA diluted 10 times. For normalization, 3 endogenous genes were used (18 s rRNA, GAPDH and β-actin). The references of the mixture of primers and probes used to amplify each one of the genes under study were as follows: caspase-3 (Mm01195085_m1), caspase-1 (Mm00438023_m1), NCS-1 (Mm00490552_m1), Rrad (Mm00451053_m1), 18 s rRNA (Hs99999901), GAPDH (4352932E) and β-actin (4352933E), wherein the number between parenthesis corresponds to the TaqMan® assay identification numbers of the genes measured.

All the PCR reactions were carried out in an ABI Prism 7000 Sequence Detection System thermocycler (Applied Biosystems). The thermal cycle parameters were as follows: incubation at 95° C. during 10 min and 40 cycles of 95° C. during 15 s and 60° C. during 1 min. The relative expression of caspase-3, caspase-1, NCS-1, and Rrad was normalized by applying the geometric mean value of the three endogenous genes.

### 1.6 Spinal Cord Protein Extraction and Western Blot Analysis

The spinal cord samples of wild type mice and SOD1G93A mice treated with TTC were homogenized in liquid nitrogen with the extraction buffer consisting of 150 mM NaCl, 50 mM Tris-HCl pH7.5, 1% desoxycholate, 0.1% SDS, 1% Triton X-100, 1 mM NaOVa, 1 mM PMSF, 10 μg/mL leupeptin and aprotinin and 1 μg/mL pepstatin. It was centrifuged at 4° C., during 10 minutes at 3,000×g. After quantifying the concentration of protein in the supernatant of each sample using the BCA method (9643 Sigma), 25 μg of protein were loaded in a gel at 10% of acrylamide. PVDF membranes were used for the transfer, which were blocked with TTBS solution at 5% skimmed milk (20 mM Tris base, 0.15M NaCl, pH=7.5, 0.1% Tween) during one hour. Later they were incubated with the primary antibody all night at 4° C. (anti-GAPDH (sc-25778, Sta. Cruz)).

Following incubation with the primary antibody, the membranes were washed with TTBS and incubated with the secondary antibody for 1 hour at room temperature. Finally, there was revelation by chemiluminescence (Western Blotting Luminol Reagent, sc-2048 Sta. Cruz). The films were scanned and analyzed using the AlphaEase FC (Bonsai Technologies). The statistical analysis was carried out using the ANOVA test and the Student-Neuman-Keuls test.

### Results

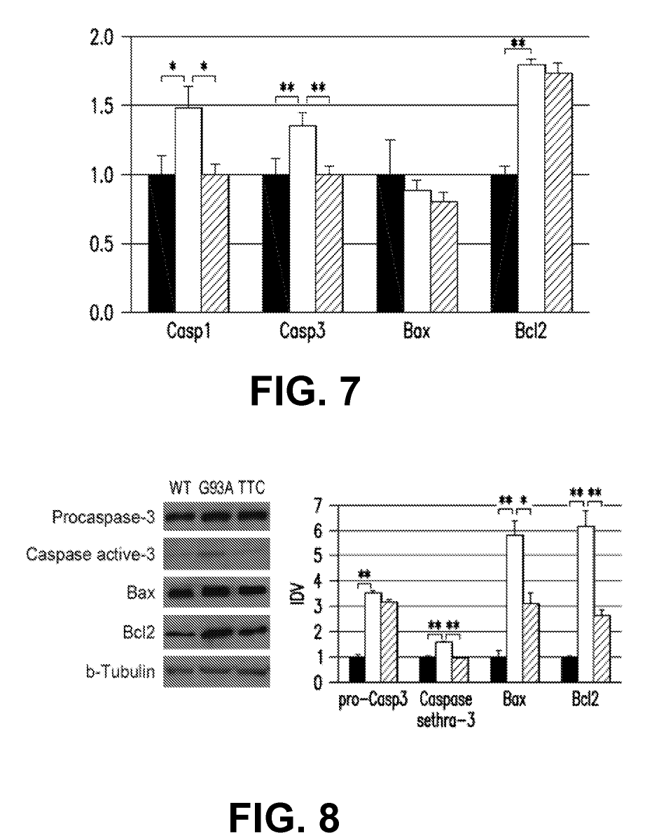

One of the effects of ALS is the degeneration of the motor neurons, i.e., neurons innervating muscle and responsible in part for muscle function. The transcriptional study at the level of the spinal cord of these mice, of symptomatic age, appears in FIG. 7, comparing the transcriptional regulation of the genes caspase-1 (P<0.05), caspase-3 (P<0.05),and Bcl2 (P<0.01), but no significant difference was found in the expression profile of the gene Bax (P>0.05) in control SOD1G93A mice when compared to the wild type (FIG. 7).

In the group of mice that received treatment with TTC, the levels of expression of caspase-1 and caspase-3 were maintained in the wild type and significant differences were only found when they were compared to the group of untreated mice (P<0.05 and P<0.01, respectively). However, the expression of the genes Bax and Bcl2 was not affected by the treatment of TTC (P>0.05) in the spinal cords of these transgenic mice (FIG. 7)

In order to evaluate the effects of TTC on the mechanisms that reverse apoptosis which can induce cell death in the spinal cord of the SOD1G93A mice, a protein study was also carried out. The data revealed that the activation of the caspase-3 gene (P<0.05) decreased perceptibly in the mice treated with TTC in relation to the control group, reaching similar levels to those of wild-type mice, whereas the levels of the pro-caspase-3 protein were not affected in the transgenic animals. In contrast to the results obtained from the expression analysis, in the Western blot it was observed that the proteins Bax and Bcl2 were in lesser amounts in the mice treated with TTC (FIG. 8).

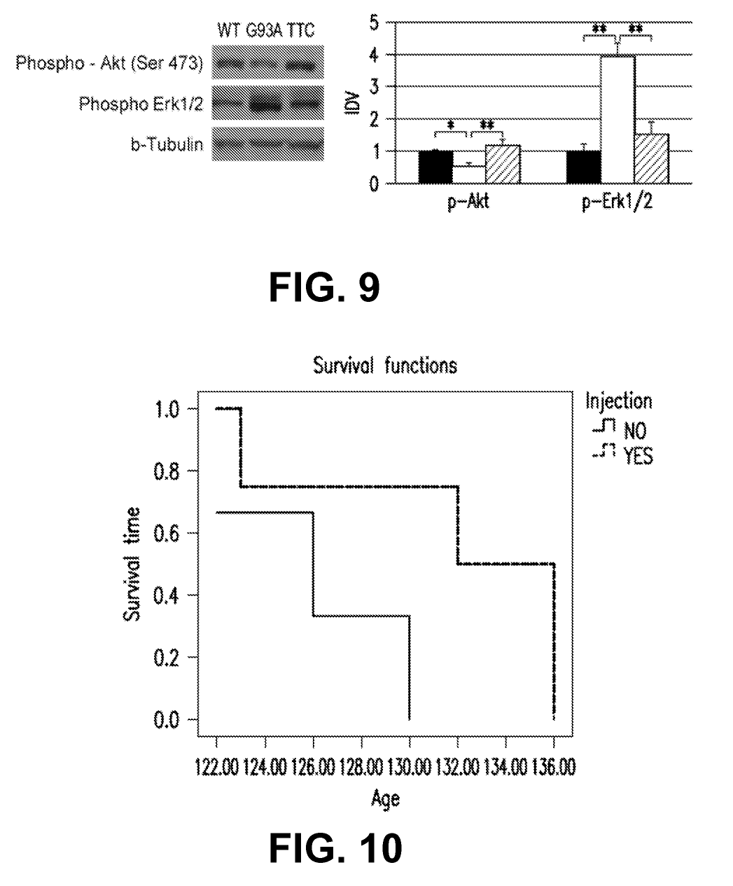

An action mechanism of TTC is the phosphorylation of Akt, a kinase protein that is activated by various growth factors involved in the blocking of routes mediated by phosphatidylinositol 3-kinase. Gil et al. Biochem. J. 373: 613-620 (2003). The densitometric quantification indicated that the animals treated with TTC had more than two times the levels of Akt phosphorylated in Ser473 when they were compared to the controls of the empty vector (P<0.05), as determined by the Western blot analysis through the use of phospho-specific antibodies (FIG. 9).

The equimolar charge of proteins was confirmed by detection with anti-tubulin antibodies. The phosphorylation of ERK1/2 by TTC in cultivated cortical neurons has been previously described. Gil et al. Biochem. J. 373: 613-620 (2003). To confirm the implication of TTC in the MAP kinase route, Western blot analyses were carried out on the spinal cord extracts of the treated and untreated SOD1G93A mice of 110 days of age. The results showed a growing activation of ERK1/2 in control mice when compared to the group treated with TTC (FIG. 9), but the level of expression was similar to that of the wild-type mice.

### Example 3

Administration of a TTC Polypeptide through Intraperitoneal Injection

### Materials and Methods

### 1.1 Extraction of the TTC Polypeptide

The TTC polypeptide used corresponded to the C-terminal domain of the heavy chain of the tetanus toxin and comprised 451 amino acids (SEQ ID NO. 2). TTC was obtained according to the method described by Gil et al., 2003.

### 1.2 Transgenic Mice

The transgenic mice that overexpress human SOD1 with the mutation G93A (B6SJL-TgN[SOD1-G93A]1Gur) were obtained from The Jackson Laboratory (Bar Harbor, ME). Hemizygote mutants were used in all experiments (a mutant male mated with a non-transgenic female). The transgenic mice were identified by PCR amplification of the DNA extracted from the tail, as described in Gurney et al. (1994). The animals were kept in the Mixed Research Unit of Zaragoza University. They were given food and water ad libitum. All experiments and care of the animals were conducted in compliance with the rules of Zaragoza University and of the international guide for the care and use of laboratory animals.

### 1.3 Intraperitoneal Injection of the TTC Polypeptide in the Animals

At the age of 12 weeks intraperitoneal injections were given to the transgenic SOD1G93A mice with 250 μL at a concentration of 0.5 μM of the TTC polypeptide. The injection was repeated weekly throughout life.

### 1.4 Measurement of the Survival of the Animals.

The end point in the life of the mice was considered to be when the animal placed in a supine position was unable to turn itself around.

### Results

### 2.1 TTC Prolongs the Survival of Transgenic SOD1G93A Mice

As can be seen from FIG. 10 and TABLE 2, maximum survival was detected in the mice from the group treated with TTC, which reached an average of 135 days; 9 more than the control group.

TABLE 2
Survival data of the control group and of the group treated with TTC

| | Control (n = 3) | TTC (n = 3) | P value |
|---|---|---|---|
| Mortality | 126 ± 4 | 135 ± 2 | 0.021 |

### Example 4

### Administration of TTC Causes Changes in the Expression of Genes Related to the Calcium in the Spinal Cord

Neuron protein NCS1 regulates neurosecretion in a calcium-dependent manner (McFerran et al. J. Biol. Chem. 273:22768-22772 (1998)) and it has also been related to the modulation of the calcium/calmodulin dependent enzymes involved in the neuronal signal transduction (Schaad et al. Proc. Natl. Acad. Sci. USA 93:9253-9258 (1996)). The expression of NCS1 was tested using tissues from the spinal cord of SOD1G93A mice 50 days after treatment with TTC.

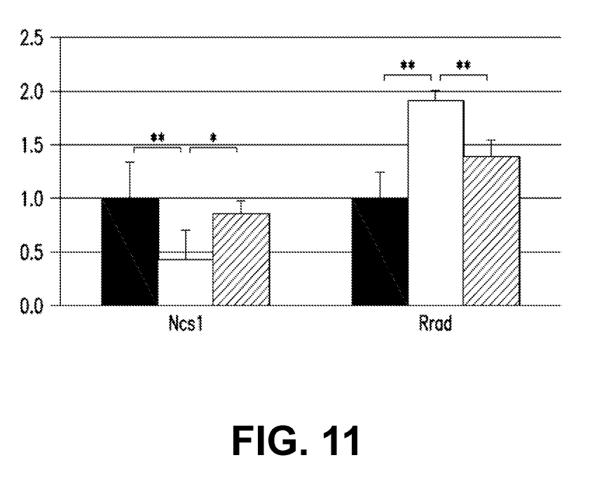

In the RT-PCR experiments it was found that the expression of the NCS1 gene was repressed (P<0.05) in the transgenic mice with late symptoms in relation to the wild-type mice of the same age. At the same time, the mice that received the intramuscular treatment with TTC had higher levels of NCSI (P<0.05), approaching those of the wild type. With the same samples, the levels were measured of messenger RNA of the gene related to Ras and associated the diabetes gene (Rrad). This example shows that the levels of Rrad were increased almost twice in the spinal cord of the control transgenic mice when compared to wild-type mice of similar age. However, in comparison to the control mice, the treatment with TTC in SOD mice perceptibly reduced the expression of Rrad (P<0.05), reaching similar values to those obtained in the wild-type mice (FIG. 11).

### Example 5

### Protective Effect of TTC on Neuromuscular Function

### Materials and Methods

### 1.1 Construction of Recombinant Plasmid Carrying TTC DNA.

A TTC-encoding gene was cloned into the pcDNA3.1 (Invitrogen S. A., Prat de Llobregat, Spain) eukaryotic expression plasmid under control of the cytomegalovirus (CMV) immediate-early promoter. The TTC gene was removed from pGex-TTC plasmid (Ciriza et al., 2008a) with BamHI and NotI restriction enzymes and inserted into pCMV to create the pCMV-TTC plasmid. After sequencing, vectors were expanded in chemically competent Escherichia coli (DH5α) and purified using GENELUTE® maxiprep-kit (Sigma-Aldrich Quimica, S.A., Madrid, Spain).

### 1.2 Transgenic Mice.

Transgenic mice with the G93A human SOD1 mutation (B6SJL-Tg[SOD1-G93A] 1Gur) were purchased from The Jackson Laboratory (Bar Harbor, ME, USA). Hemizygotes were maintained by breeding SOD1G93A males with female littermates. The offspring were identified by PCR amplification of DNA extracted from the tail tissue, as described in The Jackson Laboratory protocol for genotyping hSOD1 transgenic mice (available at jaxmice.jax.org/pub-cgi/protocols.sh?objtype=protocol,protocol_id=523). Mice were housed in the Unidad Mixta de Investigacion of the University of Zaragoza. Food and water were available ad libitum. All experimental procedures were approved by the Ethics Committees of the institutions and followed the international guidelines for the use of laboratory animals based on the guidelines for the preclinical in vivo evaluation of pharmacological active drugs for ALS/MND.

### 1.3 Electrophysiological Tests.

Two groups of male SOD1G93A mice were injected with recombinant plasmid pCMV-TTC or empty plasmid in the hind paw. To assess neuromuscular function, nerve conduction tests were performed at 12 and 16 weeks of age. A third group of age-matched wild-type mice (n=8) was also tested for comparisons. For motor nerve conduction tests, the sciatic nerve was stimulated percutaneously with a pair of needle electrodes placed near the sciatic notch, and the compound muscle action potential (CMAP, M wave) was recorded from tibialis anterior and plantar muscles with microneedle electrodes.

For sensory nerve conduction tests, the recording electrodes were placed near the digital nerves of the fourth toe to record the compound sensory nerve action potential (CNAP). The evoked potentials were amplified and displayed on a digital oscilloscope (Tektronix 450S) at appropriate settings to measure the amplitude from baseline to the maximal negative peak and the latency from stimulus to the onset of the first negative deflection (Navarro et al. Exp. Neurol. 129:217-224 (1994); Verdu et al. Exp Neurol 129:217-224 (1994); Udina et al. Glia 47:120-129 (2004)). During 7 electrophysiological tests, the animals were placed over a warm flat steamer controlled by a water circulating pump to maintain body temperature.

### Results

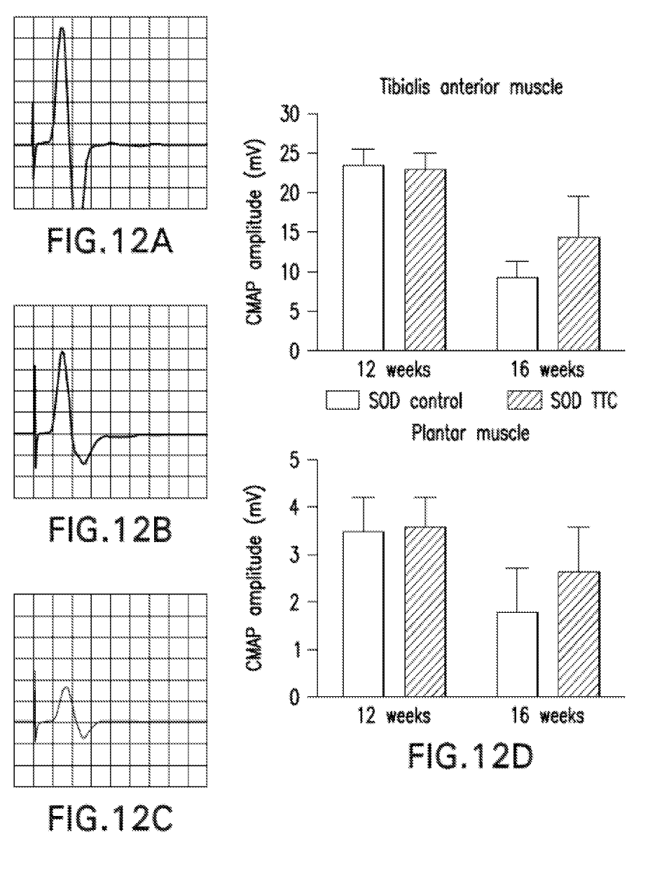

The neuromuscular function of SOD1G93A mice was assessed at two time points: at 12 weeks of age, just before the approximate time of disease onset, and 16 weeks of age, when the disease is in a late symptomatic stage. By 12 weeks of age, there were marked abnormalities in motor nerve conduction tests, evidenced by a 40%-50% decline in the amplitude of the M waves in tibialis anterior and plantar muscles of both TTC-treated and vehicle-plasmid transgenic mice (FIG. 12, TABLE 3).

TABLE 3
Neurophysiological results in the groups of wild-type (WT), SOD1G93A control
(SOD control), and SOD1G93A TTC-treated (SOD + TTC) mice. Values are mean ± SEM

| | | 12 weeks | | | 16 weeks | | |
|---|---|---|---|---|---|---|---|
| | | Group | | | | | |
| | | WT | SOD control | SOD + TTC | WT | SOD control | SOD + TTC |
| | | (n) | | | | | |
| | | (8) | (7) | (7) | (8) | (3) | (3) |
| Tibialis ant | Latency (ms) | 0.94 ± 0.04 | 1.09 ± 0.04* | 1.09 ± 0.02* | 0.87 ± 0.03 | 1.13 ± 0.04* | 1.14 ± 0.04* |
| muscle | CMAP (mV) | 52.3 ± 2.4 | 23.4 ± 2.2* | 23.0 ± 2.0* | 50.4 ± 2.8 | 9.2 ± 2.1* | 14.3 ± 5.2* |
| Plantar muscle | Latency (ms) | 1.69 ± 0.04 | 1.92 ± 0.03* | 1.94 ± 0.07* | 1.55 ± 0.08 | 2.00 ± 0.10* | 2.23 ± 0.18* |
| | CMAP (mV) | 7.2 ± 0.4 | 3.5 ± 0.7* | 3.6 ± 0.6* | 7.0 ± 0.5 | 1.8 ± 0.9* | 2.6 ± 0.9* |
| Digital nerve | Latency (ms) | 1.08 ± 0.03 | 1.26 ± 0.06* | 1.17 ± 0.05 | 1.00 ± 0.06 | 1.24 ± 0.05* | 1.21 ± 0.04 |
| | CNAP (μV) | 51.7 ± 3.7 | 43.9 ± 5.3 | 41.2 ± 4.2 | 51.4 ± 3.5 | 44.6 ± 6.6 | 41.0 ± 5.4 |

*P < 0.05 vs. WT group.
CMAP, compound muscle action potential;
CNAP, compound nerve action potential.

There was also a slight but significant increase in the latency (about 14% longer) compared to age-matched wild-type mice (TABLE 3). At 16 weeks, there was a clear reduction in the M wave amplitudes in vehicle-treated SOD1G93A mice, to about 20%-25% of normal values (FIG. 12). This decline was less pronounced in TTC-treated mice (to 30%-38%), although the differences did not attain significance. The latency of M wave onset slightly increased between 12 and 16 weeks in the vehicle-treated SOD mice (TABLE 3), in contrast to the mild shortening and consequent increase in conduction velocity that occur in normal mice during this age (Verdu et al. Neurobiol. Aging 17:73-77 (1996)).

Fibrillation potentials were detected with moderate abundance in the tested muscles at 12 weeks; these were increased at 16 weeks. In contrast to motor nerve abnormalities, sensory nerve conduction tests showed no significant differences in the amplitude of CNAPs recorded from the digital nerves in the toes between groups (TABLE 3). The latency time of sensory CNAP was slightly delayed in vehicle-plasmid SOD1G93A mice compared to age-matched wild-type animals. These findings indicate that gene delivery of TTC has protective effects on the ALS murine model expressing the G93A mutant human SOD1 gene with regard to neuromuscular function.

### Example 6

TTC Protects against Spinal Motor Neuron Loss and Promotes Reduction of Microgliosis.

### Materials and Methods

### 1.1 Construction of Recombinant Plasmid Carrying TTC DNA.

A TTC-encoding gene was cloned into the pcDNA3.1 (Invitrogen S. A., Prat de Llobregat, Spain) eukaryotic expression plasmid under control of the cytomegalovirus (CMV) immediate-early promoter. The TTC gene was removed from pGex-TTC plasmid (Ciriza et al., 2008a) with BamHI and NotI restriction enzymes and inserted into pCMV to create the pCMV-TTC plasmid. After sequencing, vectors were expanded in chemically competent Escherichia coli (DH5α) and purified using Genelute maxiprep-kit (Sigma-Aldrich Química, S. A., Madrid, Spain).

1.2 Transgenic mice.

Transgenic mice with the G93A human SOD1 mutation (B6SJL-Tg[SOD1-G93A]1Gur) were purchased from The Jackson Laboratory (Bar Harbor, Me., USA). Hemizygotes were maintained by breeding SOD1G93A males with female littermates. The offspring were identified by PCR amplification of DNA extracted from the tail tissue, as described in The Jackson Laboratory protocol for genotyping hSOD1 transgenic mice (available at jaxmice.jax.org/pub-cgi/protocols.sh?objtype=protocol,protocol_id=523). Mice were housed in the Unidad Mixta de Investigacion of the University of Zaragoza. Food and water were available ad libitum. All experimental procedures were approved by the Ethics Committees of the institutions and followed the international guidelines for the use of laboratory animals based on the guidelines for the preclinical in vivo evaluation of pharmacological active drugs for ALS/MND.

### 1.3 Histological and Immunohistochemical Processing.

Male SOD1G93A mice were injected with recombinant plasmid pCMV-TTC or empty plasmid in the hind paw. To assess neuromuscular function, nerve conduction tests were performed at 16 weeks of age. Following electrophysiological tests, the animals (n=5) were perfused with 4% paraformaldehyde in PBS. The lumbar segment of the spinal cord was removed, post-fixed for 24 h, and cryopreserved in 30% sucrose. Transverse 40 μm thick sections were serially cut with a cryotome (Thermo Electron, Cheshire, UK), at L2, L3 and L4 segmental levels.

For each segment, each section of a series of 10 was collected sequentially on separate gelatin-coated slides. One slide was rehydrated for lmin with tap water and stained for lh with an acidified solution of 3.1 mM cresyl violet. Then, the slides were washed in distilled water for lmin, dehydrated, and mounted with DPX (Fluka). Motor neurons were identified by their localization in the lateral ventral horn of the stained spinal cord sections and counted following strict size and morphological criteria.

Overlapping images covering the whole lateral ventral horn were taken at 40×, and a 20 μm squared grid was superimposed onto each micrograph. Only motor neurons with diameters larger than 20 μm and with polygonal shape and prominent nucleoli were counted. The number of motor neurons present in both ventral horns was counted in four serial sections of each L2, L3 and L4 segments. Another series of sections was blocked with TBS-Triton-FBS and incubated for 2 days at 4° C. with primary antibody anti-glial fibrilar acidic protein (GFAP, 1:1000, Dako) or rabbit anti-ionized calcium binding adaptor molecule 1 (Ibal, 1:2000, Wako) to label astrocytes and microglia respectively.

After washes, sections were incubated for 1 day at 4° C. Cy3-conjugated secondary antibody (1:200; Jackson Immunoresearch). Sections from the three groups of mice were processed in parallel for immunohistochemistry. Microphotographs of the grey matter of the ventral horn were taken at 400× and, after defining the threshold for background correction, the integrated density of GFAP or Ibal labeling was measured using ImageJ software (Penas et al. J. Neurotraum 26:763-779 (2009)). The integrated density is the area above the threshold for the mean density minus the background.

### Results

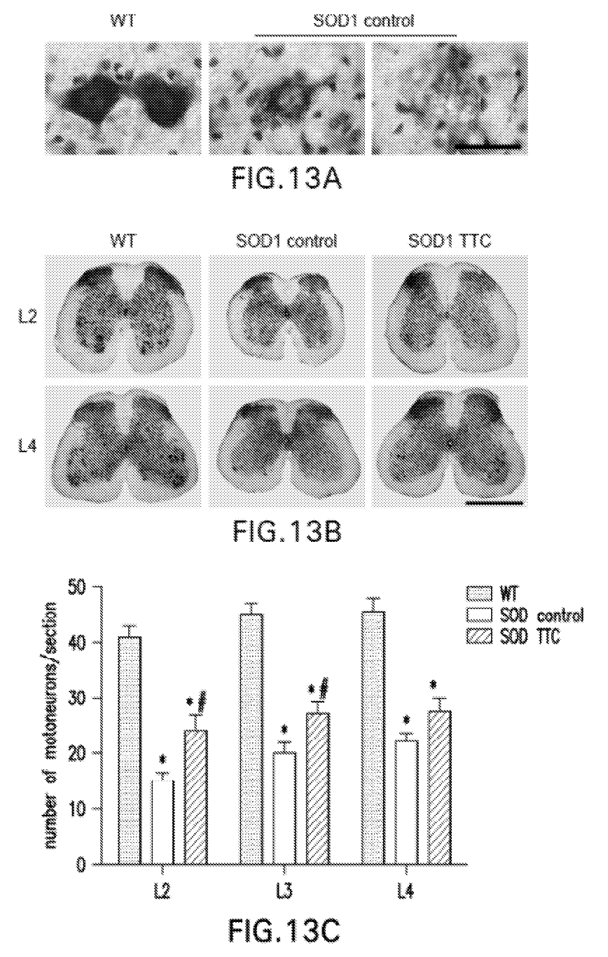

The degenerative events underwent by SOD1G93A mice motor neurons were observed under light microscopy. A prominent feature of the motor neurons in SOD1G93A mice was a vacuolization of the cytoplasm indicating active degeneration (FIG. 13A). These vacuoles had different sizes and a clear content. SOD1G93A mice motor neurons also showed a depletion of Nissl substance, becoming pale and less visible. In contrast, the motor neurons in wild type mice had darkly stained aggregates of Nissl substance and no cytoplasmic vacuoles (FIG. 13A). The extent of motor neurons degeneration was determined by counting the number of stained motor neurons in the lateral ventral horns of lumbar spinal cord sections of wild type and SOD1G93A mice at 16 weeks of age.

The three lumbar segments sectioned contain motor nuclei of different muscles of the hind limbs; the nuclei of quadriceps femoris muscles, in which plasmid injections were made at 8 weeks, are mainly located at L2; whereas motor nuclei of tibialis anterior and foot plantar muscles, that were tested electrophysiologically, are mostly represented at L3 and L4 levels respectively (McHanwell et al. Philos. Trans. R. Soc. Lond. B Biol. Sci. 293:477-508 (1981)). FIG. 13B shows representative spinal cord sections from wild type, control SOD1G93A mice, and SOD1G93A-TTC treated mice. Only neurons that met the criteria of a motor neuron were included in the counts. Small neurons were excluded from our counts; even if these neurons were, in fact, atrophic motor neurons they were unlikely to be functional motor neurons. The number of surviving motor neurons was significantly reduced at the lumbar spinal cord in both SOD1G93A groups compared to the wild-type age matched controls (FIG. 13C).

Nevertheless, the extent of motor neuron loss was significantly higher in vehicle-plasmid injected (about 43% of surviving motor neuron with respect to wild type mice) than in TTC-treated SOD1G93A mice (about 60%). The results indicate that the neuroprotective effect of TTC extended along spinal cord segments and not only affected the segment containing the quadriceps muscle motoneuronal pool. However, the improvement in motor neurons survival induced by TTC showed a slight gradient, since the proportion of motor neurons was increased in mice treated with TTC about 22% at L2, 16% at L3, and 12% at L4 compared with SOD1G93A control mice (FIG. 13C).

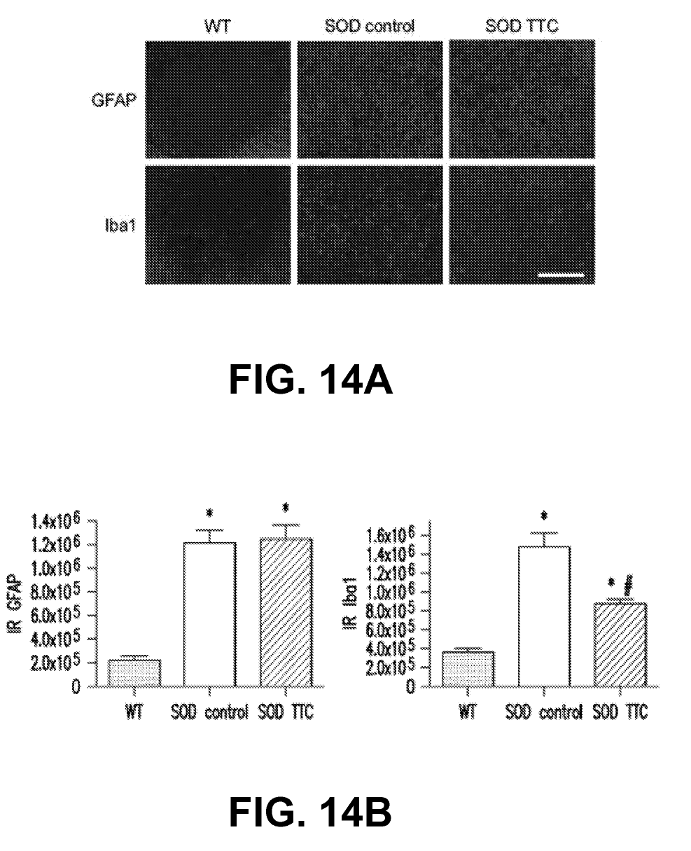

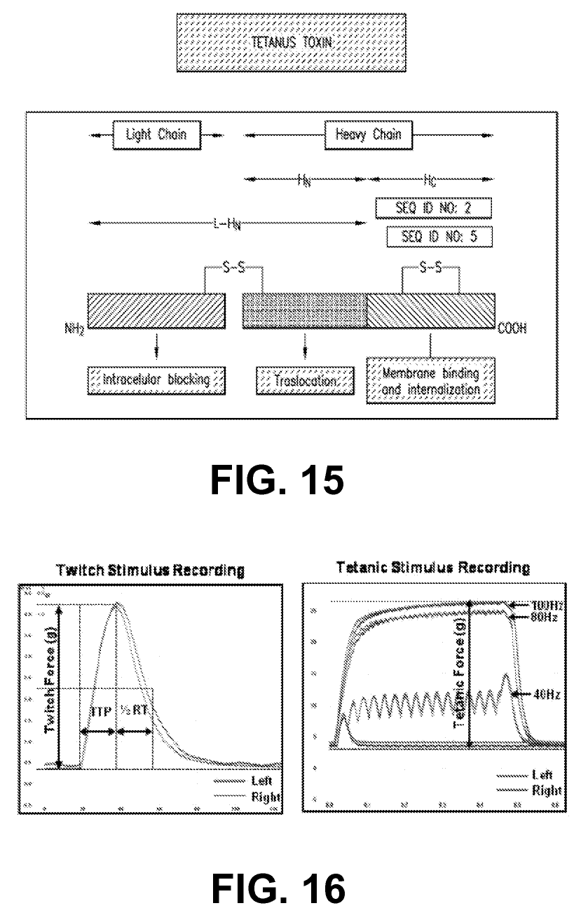

In order to indirectly examine the state of lumbar motor neurons and the reactive glial response, we stained the spinal cord sections with markers for astrocytes (GFAP) or microglia (Iba1). Glial reactivity was measured in L2 sections, as this segment had the highest increased proportion of motor neuron survival. Reactive astrocytosis and microgliosis were clearly evident in both SOD1G93A groups, at significantly higher levels than in wild type mice, which had a lower basal labelling for these markers (FIG. 14A). Quantitative analysis of the immunoreactivity showed that the TTC treatment had no effect on astrocyte reactivity, whereas it was able to promote a significant reduction of the increased microglia reactivity in the SOD1G93A mice (FIG. 14B).

### Example 7

### TTC Effect on Contractile Force

TTC, whether delivered intramuscularly as recombinant protein or expressed by plasmid (naked DNA), was observed to improve muscle contractile force in model mice.

### Materials and Methods

### 1. Naked DNA Encoding TTC

The gene encoding TTC (C-terminal domain of the heavy chain of the tetanus toxin; SEQUENCE ID NO:2) was cloned into the pcDNA3.1 (Invitrogen) eukaryotic expression plasmid under control of the cytomegalovirus (CMV) immediate-early promoter. The TTC gene was removed from pGex-TTC plasmid with BamHI and NotI restriction enzymes and inserted into pCMV to create the pCMV-TTC plasmid. After sequencing, vectors were expanded in chemically competent E. Coli (DH5) and purified using Endofree Plasmid MEGAkit (Qiagen).

All tested parameters were within the range of acceptance criteria for Drug Substance (Appearance, Concentration, Purity, Identity and size of plasmid and pDNA sequence).

### 2. TTC Protein

The protein coded as TTC (C-terminal domain of the heavy chain of the tetanus toxin; SEQUENCE ID NO:2) was produced in bioreactor using E. coli strain BL21 (DE3) in a fed-batch process at high cell density configuration. Escherichia coli BL21 cells were induced to express TTC protein by addition of isopropyl β-D-thiogalactoside (IPTG). Cells were lysed in presence of lysozyme and DNAase-I and, after a salting-out process with ammonium sulfate, protein was isolated and purified by liquid chromatography (metal-affinity, hydrophobic interaction and ion exchange). Purity and bacterial endotoxin level (Lal test) were also determined.

Previously, the gene sequence was synthesized by adapting the codon usage of E. coli and was cloned into the expression plasmid pET24b (Novagen) by restriction with NdeI and XhoI (Fermentas) enzymes. The expression vector was transmuted into the production strain BL21 DE3, with which the production was performed in the bioreactor.

### 3. Transgenic Mice

Transgenic mice that overexpress human SOD1 with the G93A mutation were obtained from the Jackson Laboratory (JAX). Female B6SJL-Tg (SOD1G93A) 1Gur/J mice were exclusively used for all analysis to eliminate gender differences and minimize the number of animals required for the study. Male B6SJL-Tg (SOD1G93A) 1Gur/J mice (purchased from JAX) were cross-bred with female Fl progeny of C57B16/J×SJL cross in order to generate all experimental female transgenic mice. Transgene expression was confirmed by PCR analysis of ear biopsy tissue taken after weaning. They were given water and food ad libitum. All the experiments were developed in accordance with the international guidelines for the use of laboratory animals.

### 4. Intramuscular Administration of Compounds

All compounds were delivered starting at day 70 after birth (symptomatic stage) by bilateral intramuscular (i.m.) injections into the following hind-limb muscles at the specified volume (total injection volume of 60 μL):
Tibialis anterior (TA)=4 μl×2
Quadriceps femoris=14 μl×2
Triceps surae (Gastroc)=12 μl×2

Although the extensor digitorum longus (EDL) muscle was not directly injected, its immediate proximity to the TA muscle ensures exposure to the injectate by diffusion. All i.m. injections were conducted under isoflurane anesthesia to ensure the accurate delivery to target muscles and to reduce needle-induced damage in awake/responsive mice and to avoid unnecessary pain. Injection volumes were kept to <15% of total muscle volume in an effort to avoid induction of pressure related muscle damage, i.e. compartment syndrome.

The doses used in the study were 300 μg (single or weekly administrated) for plasmid and 10 μg (weekly administrated) for protein.

### 5. Measurement of Muscle Contractile Force (Twitch and Tetanic Forces)

The sciatic nerve was exposed in the mid-thigh region and sectioned proximally before being placed over an electrode. Muscle length was adjusted for maximum twitch force. The sciatic nerve was then stimulated with 0.02 ms square wave pulses to record maximum single twitch force. Maximum tetanic force was determined by stimulating the sciatic nerve with trains of stimuli at 40, 80 and 100 Hz. From the maximum twitch force recorded, muscle contraction characteristics were determined by measurement of the time taken to reach peak contraction (time-to-peak; TTP) and the time to reach half relaxation CART). See FIG. 16.

### Results

### 1. Muscle Contractile Force

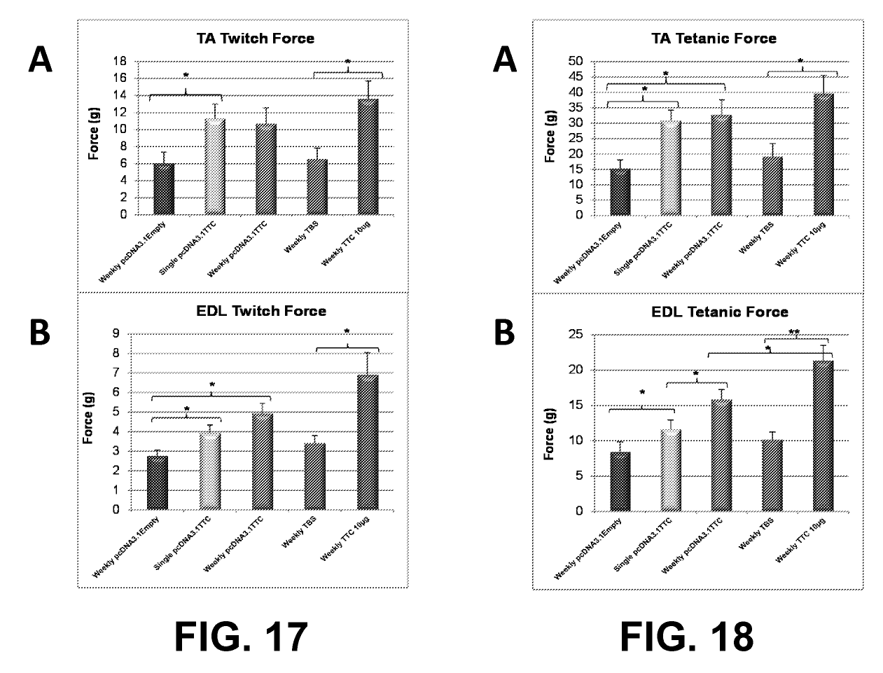

Twitch (FIG. 17) and tetanic contractile force data (FIG. 18) showed that compared to their respective controls, muscle contractile force was significantly elevated in both TA (FIG. 17A, FIG. 18A) and EDL muscles (FIG. 17B, FIG. 18B) in 120days SOD1G93A mice treated with pcDNA3.1TTC plasmid and TTC protein. Tetanic force values represent maximal muscle force generating capacity, whereas twitch values are used primarily for contractile rate characteristics. The greatest improvement in muscle contractile force was observed in TTC protein (10 μg) treated mice, which resulted in a 2.07-fold and 2.09-fold increase in tetanic force in TA and EDL muscles, respectively, compared to TBS (vehicle) treated mice. Maximum TA and EDL contractile force was also increased in pcDNA3.1TTC plasmid (weekly i.m. injection) treated mice, by 2.15-fold and 1.88-fold, compared to pcDNA3.1Empty plasmid treated mice. Interestingly, weekly i.m. injection of pcDNA3.1TTC plasmid did not result in a significant increase in TA tetanic force compared to mice that only received a single injection, however, a modest but significant difference (p=<0.04) was observed in EDL muscle tetanic force.

### 2. Muscle Contractile Characteristics

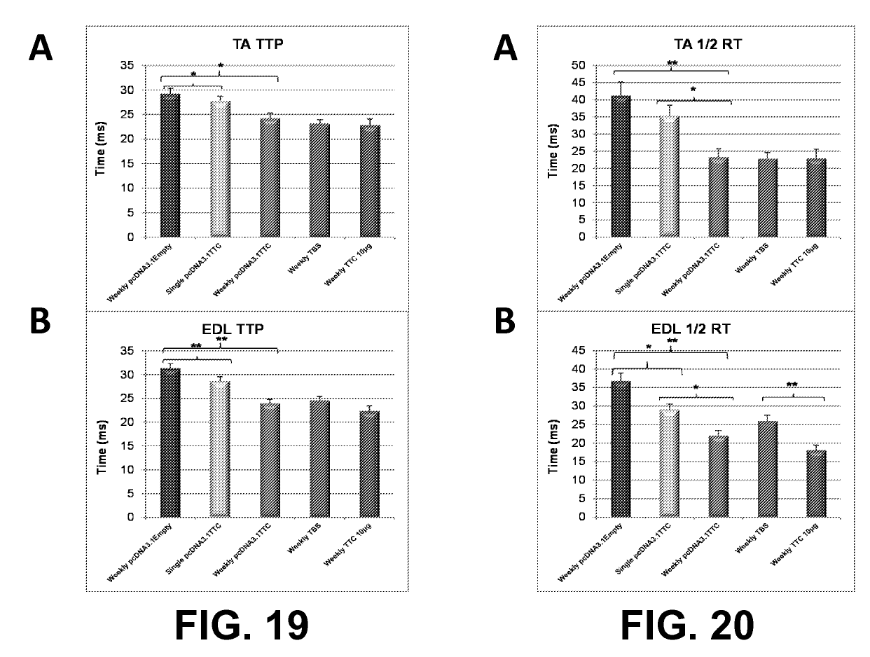

Muscle contractile characteristics data indicated that treatment with pcDNA3.1TTC plasmid (weekly i.m. injection) results in a significant improvement in TTP (FIG. 19) and 1/2RT values (FIG. 20) for both TA (FIG. 19A, FIG. 20A) and EDL muscles (FIG. 19B, FIG. 20B), compared to pcDNA3.1 Empty plasmid (weekly i.m. injection) treated controls. Additionally, the data of muscle contractile characteristics showed that weekly administration of pcDNA3.1TTC plasmid exerted a greater effect compared to single administration at 70d in SOD1G93A mice. A similar, or even greater, improvement was also observed in TTC protein (10 μg, weekly i.m. injection) treated mice, however, the TBS (vehicle) treated controls exhibited faster than expected muscle contractile characteristics.

### Example 8

### Increase in Force:Muscle Mass Ratio After TTC Administration

The experimental data provided herein shows that TTC, whether delivered intramuscularly as recombinant protein or expressed by plasmid (naked DNA), results in an improvement in muscle mass and muscle force: mass ratio in model mice.

### Materials and Methods

### 1. Naked DNA Encoding TTC

The gene encoding TTC (C-terminal domain of the heavy chain of the tetanus toxin; SEQUENCE ID NO:2) was cloned into the pcDNA3.1 (Invitrogen) eukaryotic expression plasmid under control of the cytomegalovirus (CMV) immediate-early promoter. The TTC gene was removed from pGex-TTC plasmid with BamHI and NotI restriction enzymes and inserted into pCMV to create the pCMV-TTC plasmid. After sequencing, vectors were expanded in chemically competent E. Coli (DH5) and purified using Endofree Plasmid MEGAkit (Qiagen).

All tested parameters were within the range of acceptance criteria for Drug Substance (Appearance, Concentration, Purity, Identity and size of plasmid and pDNA sequence).

### 2. TTC Protein

The protein coded as TTC (C-terminal domain of the heavy chain of the tetanus toxin; SEQUENCE ID NO:2) was produced in bioreactor using E. coli strain BL21 (DE3) in a fed-batch process at high cell density configuration. Escherichia coli BL21 cells were induced to express TTC protein by addition of isopropyl β-D-thiogalactoside (IPTG). Cells were lysed in presence of lysozyme and DNAase-I and, after a salting-out process with ammonium sulfate, protein was isolated and purified by liquid chromatography (metal-affinity, hydrophobic interaction and ion exchange). Purity and bacterial endotoxin level (Lal test) were also determined.

Previously, the gene sequence was synthesized by adapting the codon usage of E. coli and was cloned into the expression plasmid pET24b (Novagen) by restriction with NdeI and XhoI (Fermentas) enzymes. The expression vector was transmuted into the production strain BL21 DE3, with which the production was performed in the bioreactor.

### 3. Transgenic Mice

Transgenic mice that overexpress human SOD1 with the G93A mutation were obtained from the Jackson Laboratory (JAX). Female B6SJL-Tg (SOD1G93A) 1Gur/J mice were exclusively used for all analysis to eliminate gender differences and minimize the number of animals required for the study. Male B6SJL-Tg (SOD1G93A) 1Gur/J mice (purchased from JAX) were cross-bred with female Fl progeny of C57B16/J×SJL cross in order to generate all experimental female transgenic mice. Transgene expression was confirmed by PCR analysis of ear biopsy tissue taken after weaning. The animals were preserved at the UCL Institute of Neurology facilities. They were given water and food ad libitum. All the experiments were developed in accordance with the international guides for the use of laboratory animals.

### 4. Intramuscular Administration of Compounds

All compounds were delivered by bilateral intramuscular (i.m.) injections starting at day 70 after birth (symptomatic stage)into the following hind-limb muscles at the specified volume (total injection volume of 60 μL):
Tibialis anterior (TA)=4 μl×2
Quadriceps femoris=14 μl×2
Triceps surae (Gastroc)=12 μl×2

Although the extensor digitorum longus (EDL) muscle was not directly injected, its immediate proximity to the TA muscle ensures exposure to the injectate by diffusion. All i.m. injections were conducted under isoflurane anesthesia to ensure the accurate delivery to target muscles and to reduce needle-induced damage in awake/responsive mice and to avoid unnecessary pain. Injection volumes were kept to <15% of total muscle volume in an effort to avoid induction of pressure related muscle damage, i.e. compartment syndrome.

The doses used in the study were 300 μg (single or weekly administrated) for plasmid and 10 μg (weekly administrated) for protein.

### 5. Measurement of Muscle Contractile Force (Twitch and Tetanic Forces)

The sciatic nerve was exposed in the mid-thigh region and sectioned proximally before being placed over an electrode. Muscle length was adjusted for maximum twitch force. The sciatic nerve was then stimulated with 0.02 ms square wave pulses to record maximum single twitch force. Maximum tetanic force was determined by stimulating the sciatic nerve with trains of stimuli at 40, 80 and 100 Hz. From the maximum twitch force recorded, muscle contraction characteristics were determined by measurement of the time taken to reach peak contraction (time-to-peak; TTP) and the time to reach half relaxation CART). See FIG. 17 and FIG. 18.

### Results

TA muscle mass was found to be significantly increased in pcDNA3.1TTC plasmid (both single and weekly i.m. injection) and TTC protein (10 μg, weekly i.m. injection) treated mice, compared to their respective controls (FIG. 22A). EDL muscle mass was also significantly increased in pcDNA3.1TTC plasmid (weekly i.m. injection) treated mice, although the effect of TTC protein treatment was smaller (FIG. 22B). This may reflect the fact that the TA muscle is typically more severely affected at an earlier stage than the EDL muscle in SOD1G93A mice and due to the small size of the EDL muscle, thus any changes in mass are generally more subtle than those observed in TA.

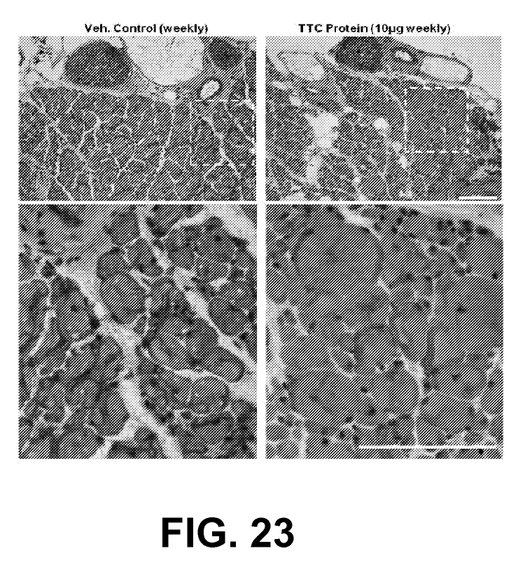

Microscopy data also shows the increase in muscle mass following TTC injection (FIG. 23). Superficial triceps surae muscle from 120d SOF1G93A mice were treated with vehicle or TTC protein (10 μg, weekly i.m. injection). The micrographs showed highly hypertrophied muscle fibers in the TTC-treated muscle, which were absent in the vehicle treated muscle from the same location within the triceps surae.

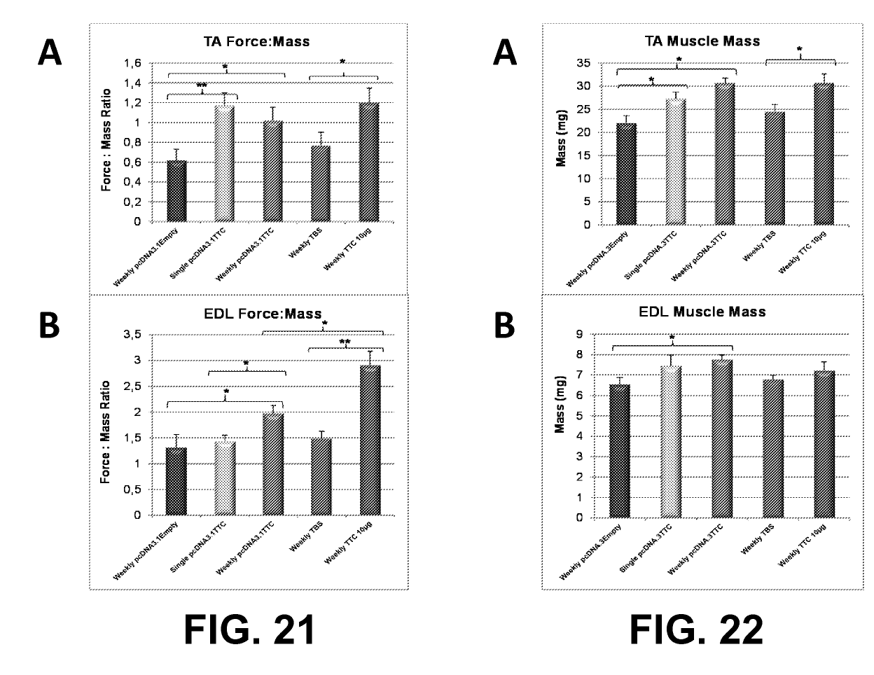

Force:Mass ratio data demonstrated that both pcDNA3.1TTC plasmid (weekly i.m. injection) and TTC protein (10 μg, weekly i.m. injection) treatment resulted in a significant improvement in TA and EDL muscle function, compared to their respective controls (See FIG. 21A and FIG. 21B). Single dose i.m. administration of pcDNA3.1TTC plasmid significantly increased TA muscle Force:Mass ratio, however, only weekly administration of pcDNA3.1TTC plasmid significantly increased the Force:Mass ratio in the EDL muscle. Additionally, weekly administration of TTC protein (10 μg) exerted a significantly greater effect on Force:Mass ratio compared to weekly pcDNA3.1TTC plasmid delivery in the EDL muscle but not for the TA muscle.

### Example 9

### Muscle Biomarkers Assessment

The intraperitoneal treatment with TTC protein in blighted/wasted muscle (EDL) was shown to bring expression levels of the markers of ALS disease progression closer to values of healthy animals and decreases oxidative stress. On the other hand, TTC treatment raised the expression of genes that are related to muscle integrity in muscles that have greater resistance to the disease.

Based on skeletal muscle biopsies, it has been proposed that Mef2c, Gsr, Col19a1, Calm1 and Snx10 are potential genetic biomarkers of longevity in transgenic SOD1G93A mice. See Calvo et al. PLoS ONE 7(3): e32632 (2012), which is herein incorporated by reference in its entirety. A significant upregulation of transcriptional levels was found in all of the genes from early asymptomatic to terminal stages, except for Calm1.

Fast extensor digitorum longus (EDL) and slow soleus muscles were used to study the expression of gene biomarkers in SOD mice after treatment with TTC. Selection of these tissues was based on previous observations that in presymptomatic SOD1G93A mice, there was no detectable peripheral dysfunction, providing evidence that muscle pathology is secondary to motor neuronal dysfunction but, at disease endstage, single muscle fiber contractile analysis demonstrated that fast-twitch muscle fibers and neuromuscular junctions are preferentially affected by ALS-induced denervation, being unable to produce the same levels of force when activated by calcium as muscle fibers from their age-matched controls. See Atkin et al. Neuromuscular Disorders 15: 377-388 (2005).

### Materials and Methods

### 1. Transgenic Mice

Transgenic mice Transgenic mice with the G93A human SOD1 mutation (B6SJL-Tg[SOD1-G93A]1Gur) were purchased from The Jackson Laboratory (Bar Harbor, Me., USA). Hemizygotes were maintained by breeding SOD1G93A males with female littermates. The offspring were identified by PCR amplification of DNA extracted from the tail tissue, as described in The Jackson Laboratory protocol for genotyping hSOD1 transgenic mice. Mice were housed according to internal procedures and food and water were available ad libitum. All experimental procedures were approved by the Ethics Committees and followed the international guidelines for the use of laboratory animals based on the guidelines for the preclinical in vivo evaluation of pharmacological active drugs for Amyotrophic Lateral Sclerosis (ALS)/Motor Neurone Disease (MND).

### 2. TTC Protein

His-tagged TTC was obtained from Escherichia coli BL21 cells previously transfected with vector encoding for (6×His)-tagged TCC as described in Herrando-Grabulosa et al. J Neurochem. 124(1):36-44 (2013), which is herein incorporated by reference in its entirety.

Escherichia coli BL21 cells were transformed with pQE3 (Qiagen, Chatsworth, Calif., USA) vector encoding for (6×His)-tagged TTC and were grown in Luria Bertani medium containing 100 mg/mL ampicillin as reported previously. Protein expression was induced by the addition of 0.4 mM isopropyl b-D-thiogalactoside (IPTG). After 3 h, cells were pelleted by centrifugation at 4000 g for 20 min at 4° C., re-suspended in lysis buffer (50 mM NaH2PO4, 300 mM NaCl, and 1% Triton-X-100; pH 8) and sonicated on ice for six 30 s periods. The suspension was centrifuged at 30 000 g for 30 min at 4° C. The clear supernatant, which contains the His-tagged protein, was purified by cobalt affinity chromatography. Mixed proteins were injected in a Fast Protein Liquid Chromatography (FPLC), which contains a cobaltagarose resin (TALON Metal Affinity resin; Clontech Laboratories, Palo Alto, Calif., USA), previously equilibrated (50 mM NaH₂-PO₄.H₂O and 300 mM NaCl; pH 7). The proteins, without His-Tags, were eluted by washing the resin with elution buffer (50 mM NaH₂PO₄.H₂O and 300 mM NaCl; pH 7). TTC contained six histidines, and was retained in the resin forming a Co-complex. TTC was eluted with the elution buffer (50 mM NaH₂PO₄.H₂O, 300 mM NaCl and 150 mM Imidazole; pH 7). Fractions collected were 0.5 mL volume. The elution process can be followed with FPLC system, that measures the absorbance at 280 nm constantly.

Protein was separated by sodium dodecyl sulfate-polyacrylamide gel electrophoresis (SDS-PAGE) at 12%. Gel was stained with GelCode Blue Stain Reagent (Pierce Chemical Co., Rockford, Ill., USA) and those fractions containing purified TTC protein were dialyzed (40 mM Na₂HPO₄, 10 mM NaH₂PO₄ and 150 mM NaCl; pH 7.4), overnight at 4° C., and for 2 h with new buffer. Protein concentrations were determined using the bicinchoninic acid assay (BCA; Pierce Chemical Co.) and lyophilized. TTC was stored in aliquots at −20° C.

### 3. Intraperitoneal Administration

SOD1G93A transgenic mice were injected intraperitoneally at 60 days and 75 days of age with 10 μg of TTC/injection (volume injected was 200 μL) using an insulin syringe (25GA 5/8 Becton Dickinson SA, Madrid, Spain). Wild type mice (used as control group) were also treated with the same protocol.

### 4. Extraction of Biological Eamples

Mice (balanced for males and females) were euthanized by asphyxiation in CO₂ chamber at day 80 of age. Subsequently, tissues (soleus and extensor digitorum longus muscles) were harvested, snap-frozen in liquid nitrogen and then stored at −80° C. for vector expression detection. Tissues from wild-type age-matched mice were also extracted.

### 5. Analysis of Genic Expression

Tissues were frozen in liquid nitrogen and pulverized in a cold mortar. To determine the expression of biomarker genes in muscle fibers, total RNA was extracted from muscles homogenized according to the TRIzol Reagent protocol (Invitrogen S.A.). RNA was obtained after fractionation with chloroform, cold isopropanol and cold ethanol. Once removed remaining DNA by Turbo-DNA free kit (Ambion), complementary DNA (cDNA) was obtained by retro-transcription of RNA using SuperScript™ First-Strand Synthesis System kit (Invitrogen).

Gene expression variations in tissues due to TTC treatment were assayed by real-time PCR. Two endogenous genes (GAPDH and β-actin) were used for normalization. Primer and probe mixtures for each gene of interest were supplied by Applied Biosystems (TABLE 4). PCR reactions were carried out in an ABI Prism 7000 Sequence Detection System (Applied Biosystems).

TABLE 4
Taqman ® probes used in gene expression assays

| | | TaqMan ® probe |
|---|---|---|
| Name | Gene symbol | (part number) |
| Glutathione reductase | Gsr | Mm00833903_m1 |
| Collagen, type XIX, alpha 1 | Col19a1 | Mm00483576_m1 |
| Sorting nexin 10 | Snx10 | Mm00511049_m1 |
| Glyceraldehyde-3-phosphate | Gapdh | 4352933E |
| dehydrogenase | | |
| Actin, beta, cytoplasmic | Actb (b-actin) | 4352932E |

Normality tests and Student's t-distribution were calculated by SPSS v19.0. All values were expressed as the mean ± S.E.M. The statistical significance threshold was set at p < 0.05 (*) and p < 0.01(**). Levels were referred to wild type results.

### Results

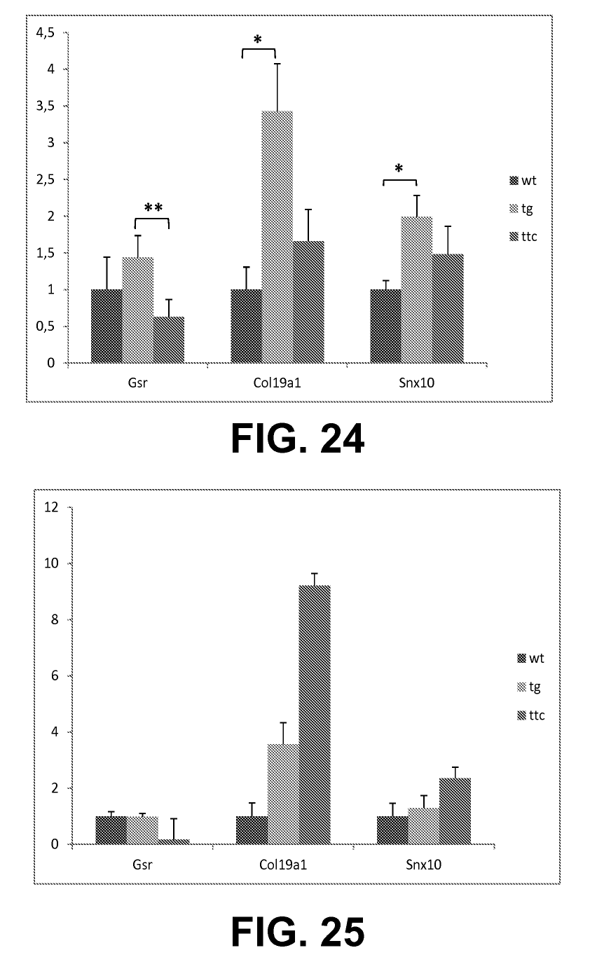

In EDL muscle (most affected in the disease and without regeneration capacity at this stage), the mRNA expression of Gsr, Col19a1 and Snx10 was reduced close to wild type values after intraperitoneal TTC treatment, which would be expected to be beneficial for the muscle since this reduction has been postulated as a predictor of longevity in transgenic SOD1G93A mice (Calvo et al. 2012) (see FIG. 24).

In the soleus muscle, the intraperitoneal injection of TTC induced an increase in the expression of Col19a1 and Snx10 in SOD1G93 mice compared to wild type mice (see FIG. 25). This result suggests that TTC could exert a different protective effect on muscle most affected by the disease progression (EDL) and muscles that are not fully affected yet (soleus). Thus, in EDL, the administration of TTC caused a reduction of the levels of the biomarkers which would be linked to prevention of the loss of muscle mass, resulting in improved survival. In soleus, TTC administration was observed to induce the expression of the biomarker proteins that are linked to muscle mass increase.

Gsr biomarker results might be related to the involvement of the TTC in the regulation of oxidative stress of the muscle in transgenic SO1G93A.

### Example 10

### Effect of TTC-Protein and TTC-Plasmid Administration on Muscle Force

### Methods

### 1. Experimental Design.

In all animals, one tibialis anterior (TA) muscle was cryolesioned. The animals (10 weeks old) were assigned to different groups (n=5) and they were cryolesioned and treated immediately after the injury, at day 7 and day 14 post injuring (protein) or immediately after the injury and at day 15 post injuring (plasmid). Measures were performed at day 15 and 30.

Investigational product was administered by intramuscular injection (0.67 μg of protein/injection or 20 μg of plasmid/injection). Phosphate buffer saline and empty (not codifying) plasmid were used as respective negative control. Non injured animals were also monitored.

Under anesthesia, the hind limbs of the mice were shaved and both tibialis anterior muscles (TA) were exposed via a 1-cm-long incision in aseptically prepared skin overlying the muscle. Traumatic freeze injury was induced by applying a 120-mm-diameter steel probe, pre-cooled to the temperature of dry ice (−79° C.), to the belly of the TA muscle for 10 seconds. After injury procedure, the skin incision was closed using 6-0 silk sutures. This procedure induced a focal injury extending distally from the spike of the tibia and spreading over approximately one-third of the muscle. The average length and maximal cross-sectional area of the lesion sites, evaluated by Evans Blue labeling, were 3202±14 μm and 3875789±27501 μm², respectively (mean±SEM). Thereafter the animals were maintained for 1 hour on a warm plate (37° C.) to prevent hypothermia.

### 2. Force Measurement.

Swiss mice were anaesthetized by intraperitoneal injection of 2.2 mg ketamine/0.4 mg xylazine/0.22 mg acepromazine per 10 g of animal body weight. Mice were put on a heating pad to maintain body and muscles at 37° C. The distal tendon of the TA muscle was attached to a FT03 Grass Instruments force transducer, which was connected to a Grass physiograph (Model 79D, Grass Technologies, Warwick, USA). An output of the polygraph was also connected to a digital data acquisition system (KCPI3104, Keithley, USA) to acquire force at a sampling rate of 5 kHz. TA was kept moist with a physiological solution containing 118.5 mM NaCl, 4.7 mM KCl, 1.3 mM CaCl₂, 3.1 mM MgCl₂, 25 mM NaHCO₃, 2 mM NaH₂PO₄, and 5.5 mM D-glucose continuously gassed with a mixture of O₂:CO₂ (95:5) to maintain a pH=7.4. Contractions were evoked every 100 seconds by field stimulation using two platinum wires 0.6 cm apart on a short section of the peroneal nerve. The platinum electrodes were connected to a Grass S88X stimulator and a Grass SIUV isolation unit (Grass Technologies, Warwick, USA). Single twitches were elicited with one 0.3 millisecond square pulse at 10 V. Maximum force was measured during a tetanic contraction with 200 millisecond train of pulses at 200 Hz.

### Results

### 1. Effect of TTC Administration on Muscle Force.

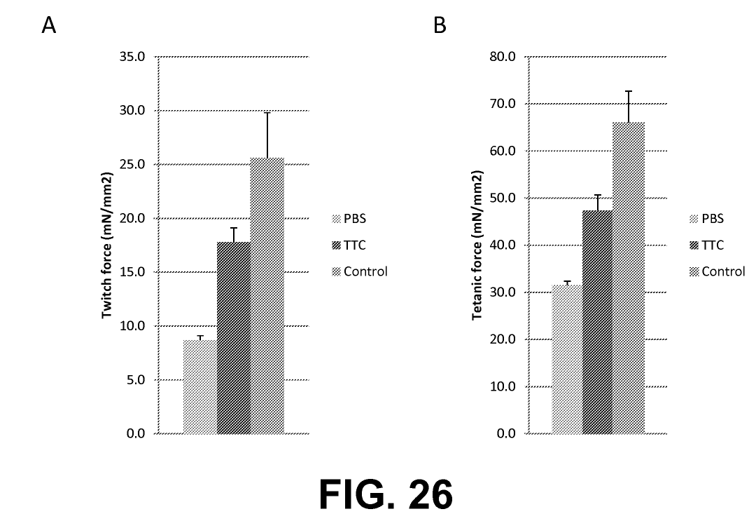

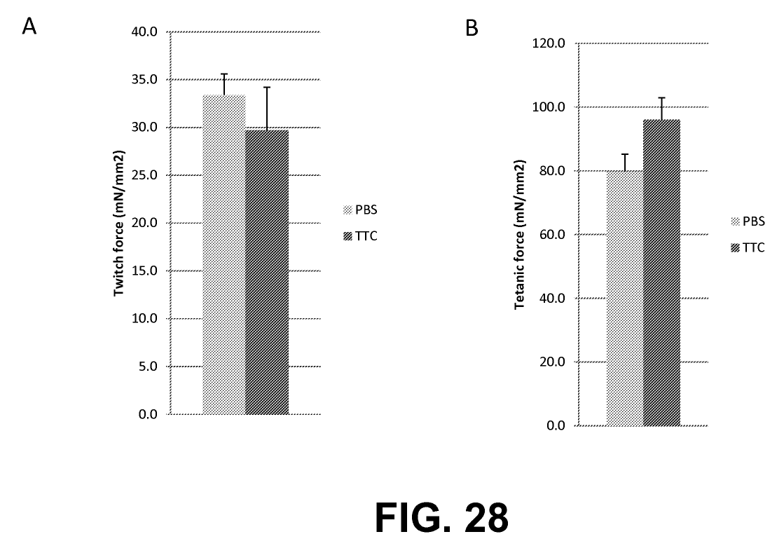

TTC or PBS (vehicle) was administrated via intramuscular injection in freeze-injured TA muscle (33.5 μg/kg body weight/7 days during 15 days). The intramuscular injection of TTC-protein caused an increase on twitch and tetanic forces at 15 days following injury (FIG. 26). Twitch force was not significantly different between freeze-injured TA muscle and muscle treated with TTC-protein at 30 days following injury (FIG. 28, panel A), but there an increase in tetanic forces (FIG. 28, panel B).

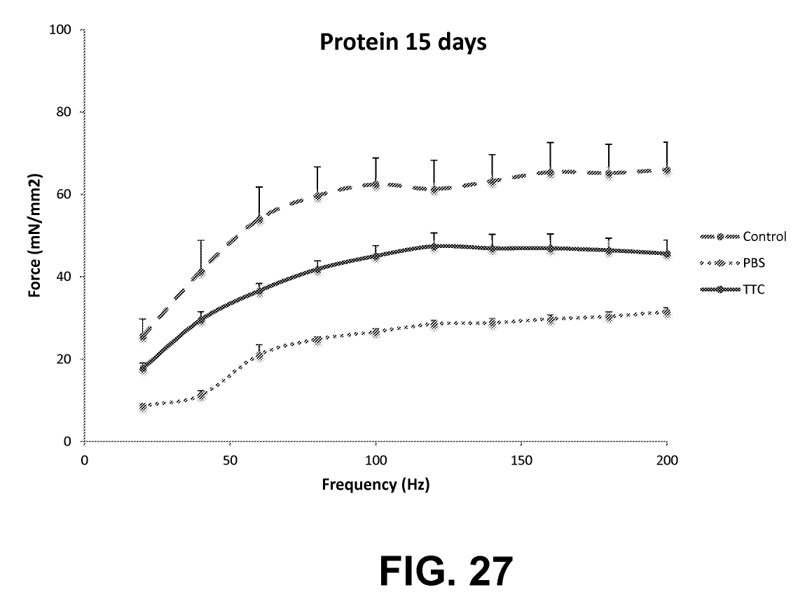

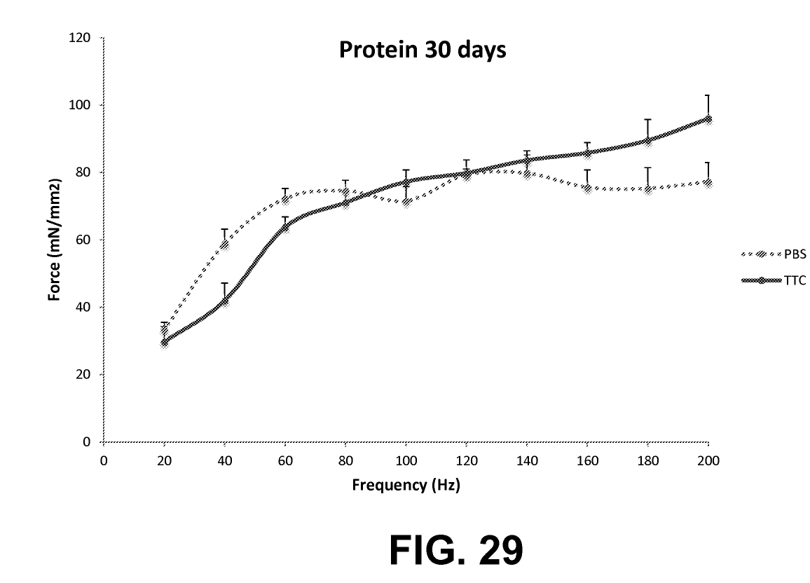

Force-frequency curves of TA muscles in TTC-treated and control (injured and PBS treated, and not injured) groups at 15 days following injury showed force value consistently higher in the TTC-protein group with respect to the injured group treated with PBS (FIG. 27). At 30 days following injury, the force-frequency curves showed that the TTC-protein treated group presented lower force values at lower frequencies (below approx. 100 Hz) but presented higher force values at higher frequencies (above approx. 100 Hz) with respect to the PBS-treated group (FIG. 29).

### 2. Effect of Plasmid-TTC Administration on Muscle Force.

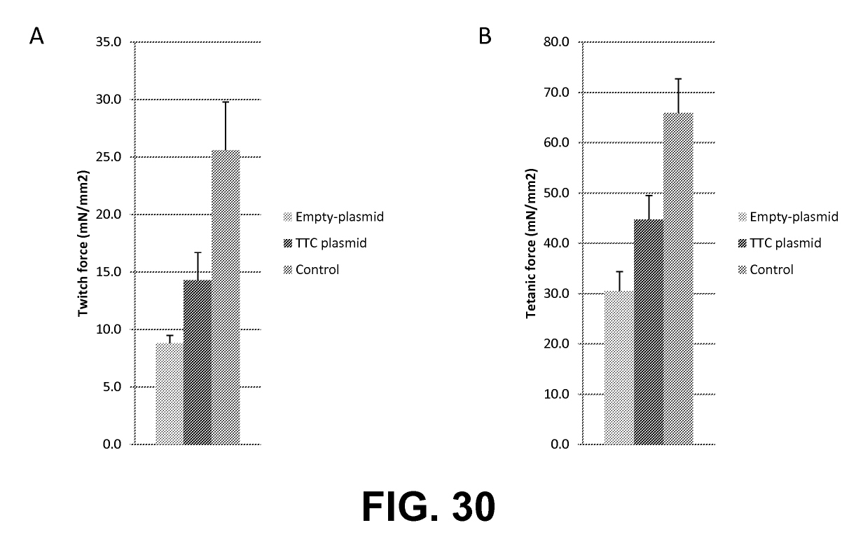

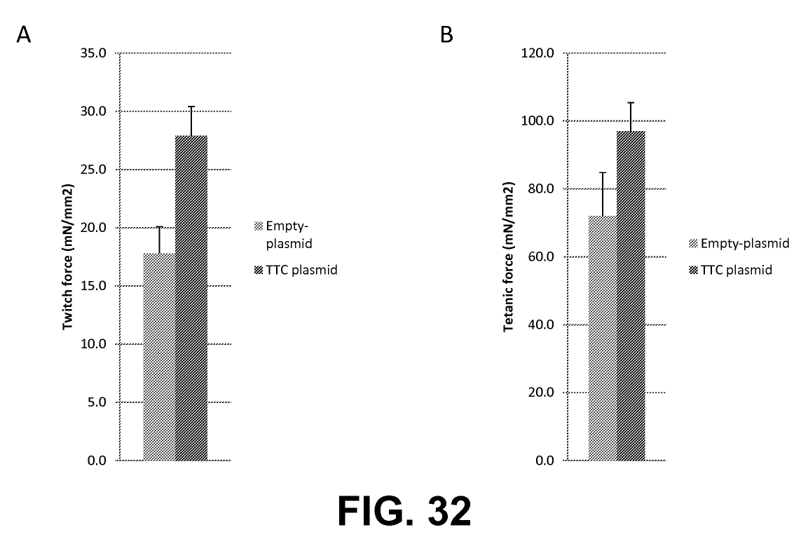

Plasmid encoding TTC or empty plasmid was administrated via intramuscular injection in freeze-injured TA muscle (post-injury single injection of lng/kg body weight during 15 days). FIG. 30 and FIG. 32 show the effect on intramuscular injection of TTC-plasmid on twitch and tetanic forces at 15 days and 30 days following injury, respectively. In both scenarios, the administration of the plasmid encoding TTC significantly increased twitch and tetanic forces with respect to the injured group treated with PBS.

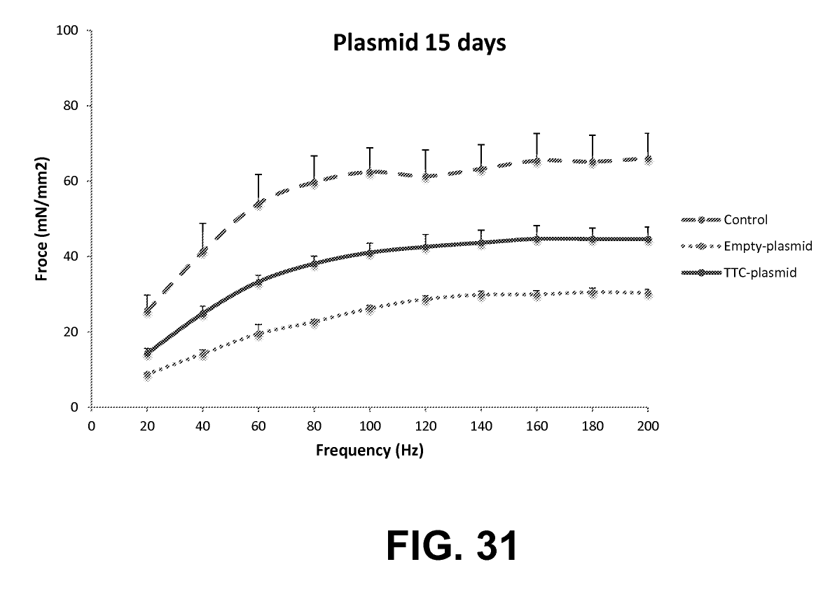

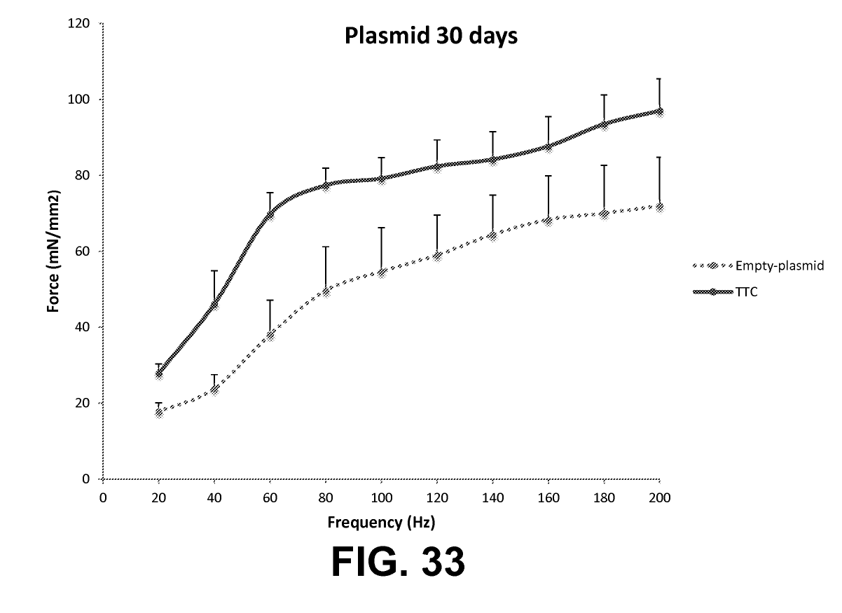

FIG. 31 and FIG. 33 show force-frequency curves of TA muscle in TTC-treated, empty-plasmid treated, and not injured (control) groups at 15 days and 30 days following injury, respectively. In both scenarios, the administration of the plasmid encoding TTC significantly increased force in TTC-plasmid treated groups with respect to the empty-plasmid treated group.

### Example 11

### Myogenic Effect of TTC: an In Vitro Study on C2C12 Myoblast Cells

### Materials and Methods

### 1. Materials

Anti-MHC and anti-myogenin antibodies were obtained from Developmental Studies Hybridoma Bank (Iowa, USA). Anti-GADPH was obtained from Abcam (Cambridge, UK). Secondary antibodies were purchased from GE-Amersham (Buckinghamshire, UK). Dulbecco's Modified Eagle Medium (DMEM), foetal bovine serum (FBS) and horse serum (HS) were obtained from Lonza (Pontevedra, SP). All other chemical reagents were from Sigma Chemical Co. (St. Louis, Mo., US).

### 2. Cell Culture and Differentiation

Mouse C2C12 myoblasts were cultured as described by the supplier (ECACC, Whiltshire, UK) through Sigma Chemical Co. Briefly, the C2C12 myoblasts were maintained in growth medium (GM) containing DMEM (4.5 g/L glucose, L-Glutamine) supplemented with 10% (v/v) foetal bovine serum (FBS), 100 U/mL penicillin, and 100 U/mL streptomycin. For routine differentiation, the cells were grown to 80% confluence and GM was replaced with differentiation medium (DM; DMEM supplemented with 2% FBS, 100 U/mL penicillin, and 100 U/mL streptomycin) for 7 days unless otherwise stated. Along this period, TTC was administrated at 1, 10 and 100 nM each 24 hours in DM.

3. Proliferation Assays using BrdU Technique.

Proliferation assays were developed using an ELISA BdrU Cell Proliferation kit (Roche; Ind., USA). Cells were cultured in 96 well plates [10.000 cells/well] in GM or GM supplemented with TTC (1, 10 and 100 nM) for 48 hours. Samples were processed according to the manufacturer's instructions. BrdU incorporation was measured using a VersaMaxPLUS reader (λ=370 nm; 21 minutes).

### 4. Inmunoblot Analysis.

Cells were stimulated with the different treatments for the indicated times at 37° C. The cell samples were directly lysed in ice-cold RIPA buffer [50 mM Tris-HCl (pH 7.2), 150 mM NaCl, 1 mM EDTA, 1% (v/v) NP-40, 0.25% (w/v) Na-deoxycholate, protease inhibitor cocktail (Sigma Chemical Co, St. Louis, Mo., US), phosphatase inhibitor cocktail (Sigma Chemical Co, St. Louis, Mo., US)]. The lysates were clarified by centrifugation (14,000×g for 15 minutes at 4° C.) and the protein concentration was quantified using the QuantiPro™ BCA assay kit (Sigma Chemical Co, St. Louis, Mo., US). For immunoblotting, equal amounts of protein were separated using SDS-PAGE and transferred onto nitrocellulose membranes. Immunoreactive bands were detected by enhanced chemiluminescence (Pierce ECL Western Blotting Substrate; Thermo Fisher Scientific, Pierce, Rockford, Ill., US). Quantification was performed using ImageJ64 analysis software.

### 5. Immunocytochemistry.

C2C12 cells were cultured and differentiated on Superfrost Plus coverslips (Thermo scientific; Braunschweig, DE). Intact cells were fixed with 4% buffered paraformaldehyde-PBS, washed, permeabilized and blocked with PBS/Triton X-100 for 30 minutes. Cells were stained with primary antibody (anti-MHC antibody) diluted in PBT overnight at 4° C. The cells were then washed and incubated with the secondary antibody in PBS/Triton X-100 for 45 minutes at 37° C. DAPI was used to counterstain the cell nuclei (Life Technologies, Invitrogen, Gran Island, N.Y., US). The digital images of the cell cultures were acquired with a Leica TCS-SP5 spectral confocal microscope (Leica Microsystems, Heidelberg, Del.). Five fields from three independent experiments were randomly selected for each treatment. Quantification of area, diameter and myonuclei aggregation was performed using ImageJ64 analysis software.

### 6. Data Analysis.

Data analysis. All values are presented as mean±standard error of the mean (SEM). Student t test were performed to assess the statistical significance of 2-way analysis. * P<0.05 was considered as statistically significant.

### Results and Discussion

### 1. Mitogenic Capacity of TTC on Myoblast C2C12 Cells.

To determine the role of TTC on the different steps of myogenesis, myoblast proliferation, and/or differentiation, the mitogenic action of TTC was explored by dose-effect experiments (1-100 nM) in proliferation conditions (GM).

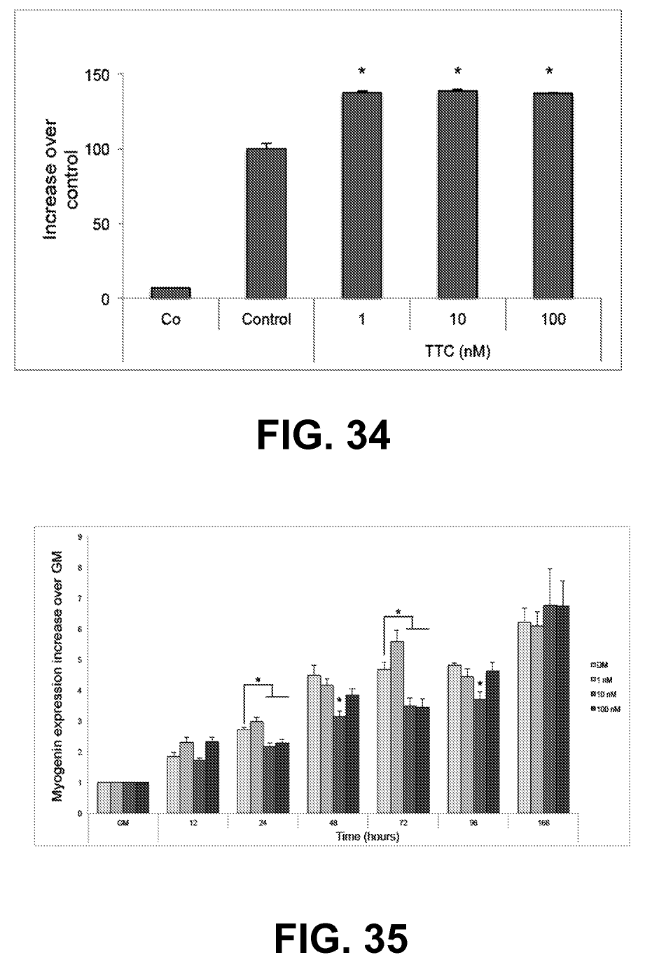

As shown in the FIG. 34, there was a significant increase for the dose tested although no significant differences among the dose tested were observed (1-100 nM). TTC treatment led to an increase of 37.7±0.7, 38.8±0.7 and 36.8±0.6 for 1, 10 and 100 nM versus control (GM), respectively. This augment, statistically significant versus control (P<0.05), showed to be maximal at 1 nM.

### 2. Myogenic Capacity of TTC on C2C12 Cells.

The myogenic program is determined by intracellular pathways that converge on a series of transcription and chromatin remodelling factors delineating the gene and microRNA expression program that delimits myogenic identity. Myogenic transcription factors are organized in hierarchical gene expression networks that are spatiotemporally activated or inhibited during lineage progression (Yin et al. (2013) Physiol Rev 93: 23-67; Tidball et al. (2010) Am. J. Physiol. Regul. Integr. Comp. Physiol. 298: R1173-R1187). In particular, myogenin is essential for myoblast lineage commitment.

To investigate whether TTC stimulated myogenesis, the C2C12 myoblasts were treated with under DM+TTC (1-100 nM) during a 7-day differentiation period. As shown in FIG. 35, the protein levels of myogenin, as detected by immunoblot, did not show a dose-dependent increase over control cells in DM. This fact ruled out the role of TTC on the myogenin expression during myogenic process. Under these conditions, the protein levels of myogenin were down-regulated in TTC-treated C2C12 cells (10 and 100 nM) compared to control cells (DM) during the early steps of the differentiation process.

Myogenic regulatory factors, in conjunction with other transcriptional regulators, induce the expression of muscle-specific genes, such as myosin heavy chain (MHC), that determine terminal myogenic differentiation (Yin et al. (2013) Physiol Rev 93: 23-67; Braun et al. (2011) Nat. Rev. Mol. Cell. Biol. 12: 349-361).

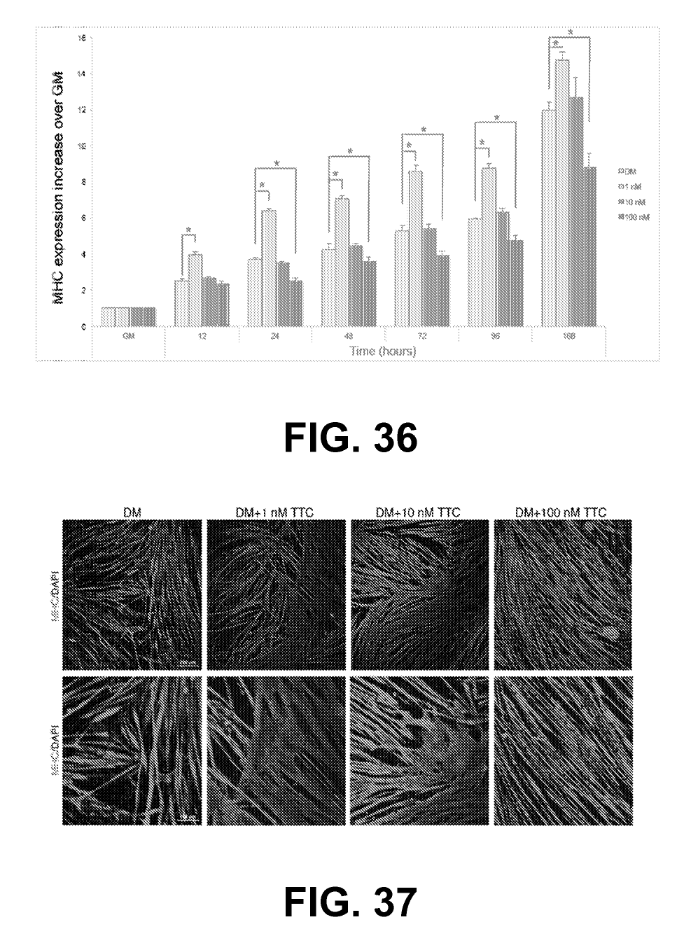

To assess the activity of TTC as a promoter of the MHC expression, C2C12 cells were switched to DM supplemented with TTC at a range of concentrations (1 to 100 nM) for 7 days. As shown in FIG. 36, the protein levels of MHC, as detected by immunoblot, were up-regulated at 1 nM compared to control differentiated cells (DM). Immunoblot analyses revealed normal protein expression of MHC at 10 nM compared to control cells. Of note, an inhibitory action on the protein expression of MHC was shown at a TTC concentration of 100 nM along the differentiation process.

### 3. Evaluation of the TTC Effect on the Differentiation Grade.

The activity of TTC on myotube hypertrophy was also evaluated. In this test, C2C12 cells were switched to DM supplemented with TTC at a range of concentrations (1 to 100 nM) for 7 days and the differentiation grade was examined by immunofluorescence (MHC/DAPI) using confocal microscopy to determine the differentiation and fusion indexes, as well as myotube area and orientation (FIG. 37).

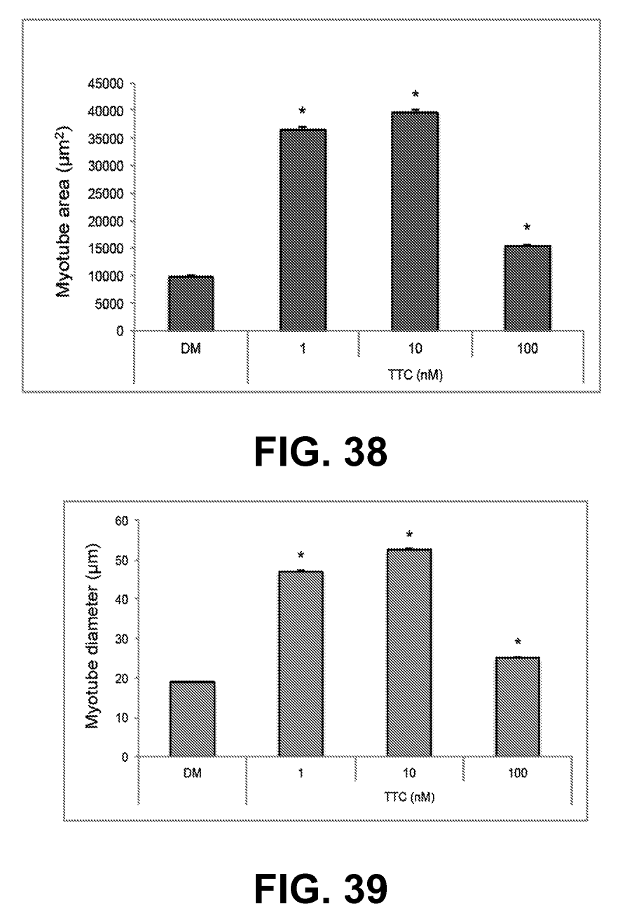

After 7 days, the myotube area (μm²) of the TTC-treated cells were significantly increased 268.7±6, 310.0±5 and 56.4±2 compared to control cells (DM) for 1, 10 and 100 nM TTC, respectively (DM: 9895.6±47.5 μm²; DM+1 nM TTC: 36488.1±550 μm²; DM+10 nM TTC: 39696.0±448 μm²; DM+110 nM: 15472.8±180 μm²) (FIG. 38).

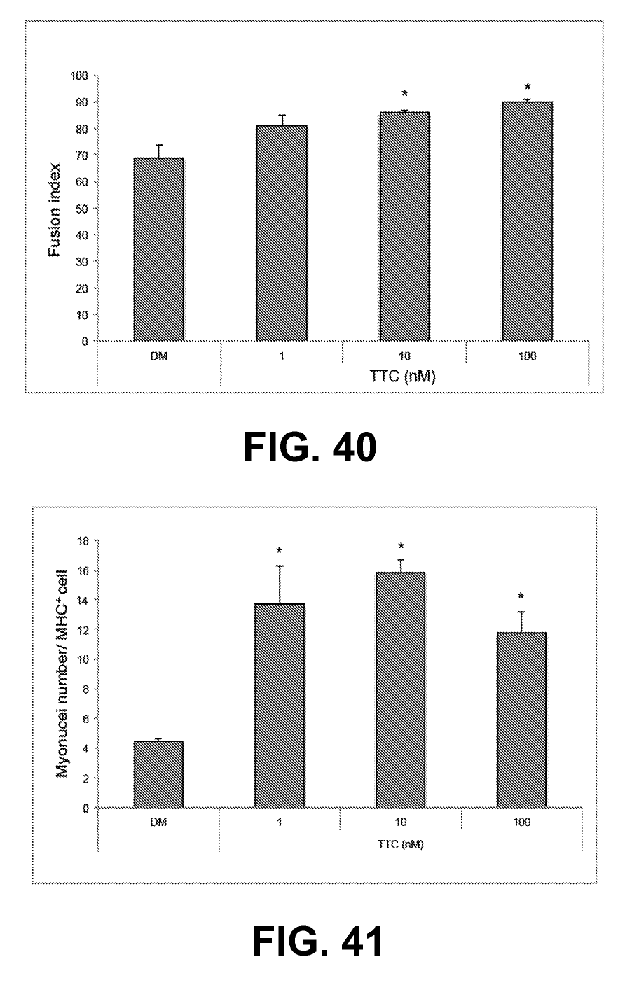

For these assays, the myotube diameters (μm) of the TTC treated cells were significantly increased 150.2±1.2, 179.0±1.4 and 33.7±0.3 compared to control cells (DM) for 1, 10 and 100 nM TTC, respectively (DM: 18.83±0.1 μm; DM+1 nM TTC: 47.1±0.2 μm; DM+10 nM TTC: 52.5±0.3 μm; DM+110 nM: 25.2±0.2 μm; FIG. 39). Moreover, fusion index showed a significant increase for 10 and 100 nM TTC compared to control cells (DM: 69±5; DM+1 nM: 81±4; DM+10 nM: 86±1; DM+110 nM: 90±1; FIG. 40).

The number of nuclei per myotube (MHC positive cell: MHC+; FIG. 41) of the TTC-treated cells was significantly increased compared to control cells (DM: 4.5±0.1; DM+1 nM TTC: 13.7±2.6; DM+10 nM TTC: 15.8±0.9; DM+1 nM TTC: 11.8±1.4). Taken together, these data showed that TTC controls the myotube growing volume at 1 and 10 nM. This is endorsed by the effective action on area, diameter, fusion index and number of myonuclei associated to the myotubes. Of note, 100 nM TTC, although showing a certain action on the myotube, showed to decrease the myotube area and diameter compared to 1 and 10 nM TTC-treated cells. However, this dose showed a significant increase on fusion index and/or the number of myonuclei associated to the myotubes.

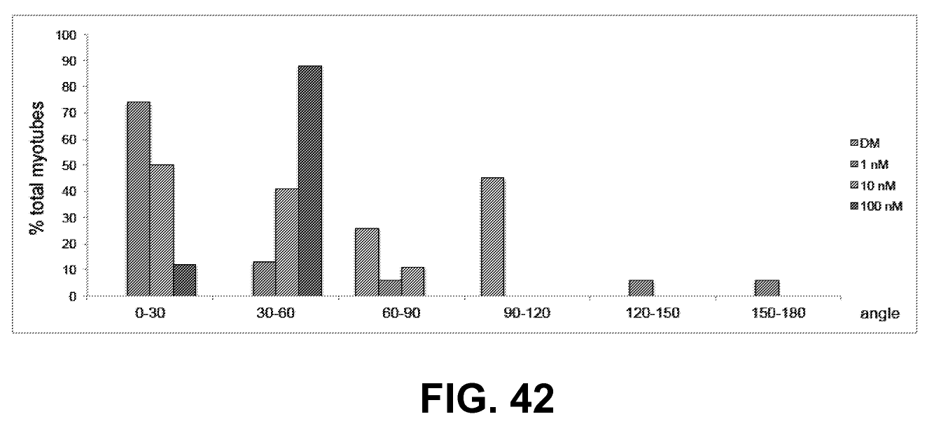

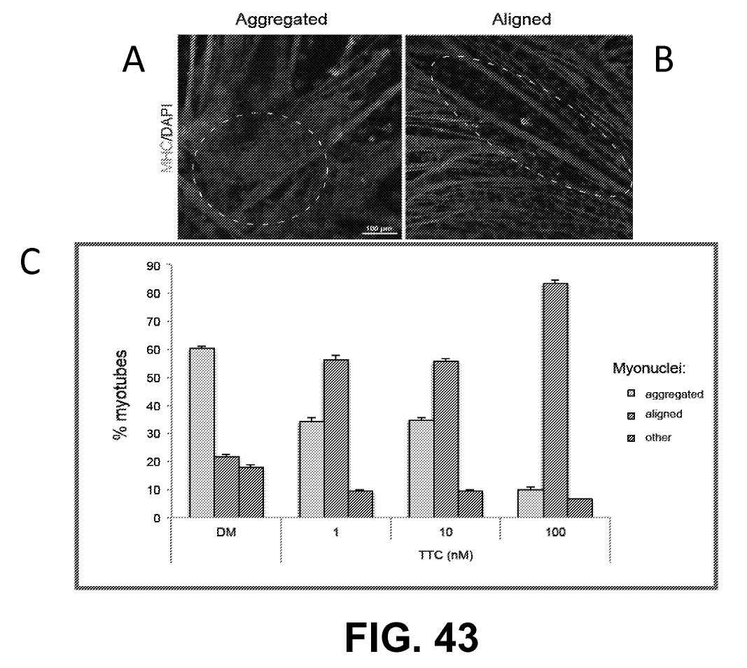

Under these conditions, a clear effect on myotube elongation was observed (FIG. 42). Indeed an estimation of myotube orientation by measurement of the angle respect to vertical axis showed a significant decrease in the dispersion of myotubes (FIG. 43). Furthermore, 83.6±1.2% of myotubes under 100 nM TTC showed nuclear distribution throughout the myotube or nuclear alignment and just 10.0±1.0% showed an aggregated nuclear distribution, which might correlate with an increase in myotube functionality (Metzger et al. (2012) Nature 484:120-124) (Percentage of total myotubes with nuclear alignment: DM: 21.8±1.0; DM+1 nM TTC: 56.3±1.4; DM+10 nM TTC: 55.8±0.8). Correct myonuclei alignment and myotube orientation exerts both structural and myogenic effects on the muscle functional output (Bian et al. (2012) Tissue Eng. 18:957-967).

The myotube population showing nuclear aggregation was 34.2±1.5 and 34.8±0.8% at 1 and 10 nM TTC, respectively, which indicates a greater effect on hypertrophy. Taken together, the experimental data indicates that TTC at low dose has a hypertrophic effect on myotubes.

### CONCLUSIONS

TTC shows an effect on C2C12 myoblast proliferation. TTC also shows a hypertrophic effect at low dose, linked to an increase of myotube area and fusion index. Furthermore, at high dose, TTC exerts a role on the proper myonuclear position and myotube orientation.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of treating a disease or condition associated with decreased muscle mass and/or muscle strength in a subject in need thereof comprising administering a therapeutically effective amount of TTC to the subject, wherein said administration is effective to (i) increase muscle mass, and/or (ii) increase muscle strength, and/or (iii) increase the rate of recovery or healing, and/or (iv) decrease fibrosis caused by said disease or condition in the subject.

2. The method according to claim 1, wherein the disease or condition is (i) a wasting disorder selected from cachexia, anorexia, muscular dystrophy, or a neuromuscular disease; or, (ii) a sequelae of immobilization, chronic disease, cancer, or injury.

3. The method according to claim 1, wherein the increase in muscle mass is (i) to compensate for wasting resulting from a wasting disorder, immobilization, or old age, or (ii) for cosmetic purposes.

4. The method according to claim 1, wherein the subject is human.

5. The method according to claim 1, wherein TTC comprises:
(a) a polypeptide comprising the sequence of SEQ ID NO:2 or SEQ ID NO:5, or a fragment, variant, or derivative thereof;
(b) a polynucleotide comprising the sequence of SEQ ID NO:1 or SEQ ID NO:6, or a fragment, variant, or derivative thereof; or,
(c) combinations thereof.

6. The method according to claim 1, wherein TTC comprises:
(a) a fusion protein or conjugate wherein a TTC polypeptide is the only therapeutic moiety;
(b) a fusion protein, or conjugate comprising at least two therapeutic moieties, wherein a TTC polypeptide is one of the therapeutic moieties;
(c) a nucleic acid encoding a fusion protein wherein a TTC polypeptide is the only therapeutic moiety;
(d) a nucleic acid encoding a fusion protein comprising at least two therapeutic moieties, wherein a TTC polypeptide is one of the therapeutic moieties; or,
(e) a combination thereof.

7. The method according to claim 1, wherein TTC is administered as a naked DNA or RNA.

8. The method according to claim 7, wherein the DNA or RNA is humanized.

9. The method according to claim 7, wherein the humanized DNA comprises the sequence of SEQ ID NO: 8, or a variant, fragment, or derivative thereof.

10. The method according to claim 7, wherein the RNA is an mRNA.

11. The method according to claim 10, wherein the mRNA is a sequence optimized mRNA.

12. The method according to claim 11, wherein the sequence optimized mRNA comprises pseudouridine (Ψ), 5-methoyxuridine (5moU), 2-thiouridine (s2U), 4-thiouridine (s4U), N1-methylpseudouridine (1mΨ), 5-methylcytidine, or a combination thereof.

13. The method according to claim 10, wherein the mRNA comprises the sequence of SEQ ID NO:9, SEQ ID NO:10, or SEQ ID NO: 11, or a fragment, variant, or derivative thereof.

14. The method according to claim 1, wherein TTC is administered at a fixed dose.

15. The method according to claim 1, wherein TTC is administered in two or more doses.

16. The method according to claim 1, wherein TTC is administered daily, weekly, biweekly, or monthly.

17. The method according to claim 1, wherein TTC is administered intramuscularly, intraperitoneally, subcutaneously, intravenously, or a combination thereof.

18. The method according to claim 1, wherein said method is performed in vivo in a mammal.

19. The method according to claim 1, further comprising at least one additional therapy.

20. The method according to claim 1, wherein the disease or condition is a muscle lesion.

21. The method according to claim 1, wherein the muscle lesion is an acute or a chronic muscle lesion.

22. The method according to claim 22, wherein the muscle lesion is a mechanical lesion, a thermal lesion, a chemical lesion, an occupational or repeated stress lesion, a iatrogenic lesion, an athletic muscle lesion, or a combination thereof.

23. The method according to claim 20, wherein the muscle lesion is treated by directly injecting a therapeutically effective amount of TTC to the site of the lesion.
